(19) United States
(12) Patent Application Publication
(10) Pub. No.: US 2023/0181905 A1
(43) Pub. Date: Jun. 15, 2023

(54) Systems, Devices, and Methods for Enhancing the Neuroprotective Effects of Non-Invasive Gamma Stimulation with Pharmacological Agents

(71) Applicant: Massachusetts Institute of Technology, Cambridge, MA (US)

(72) Inventors: Li-Huei Tsai, Cambridge, MA (US); Chinnakkaruppan Adaikkan, Bengaluru (IN); Vanesa Sarahi Fernandez Avalos, Cambridge, MA (US); Julia Maeve Bonner, Somerville, MA (US)

(21) Appl. No.: 18/064,781

(22) Filed: Dec. 12, 2022

Related U.S. Application Data
(60) Provisional application No. 63/288,046, filed on Dec. 10, 2021.
(60) Provisional application No. 63/381,855, filed on Nov. 1, 2022.

(51) Int. Cl. A61N 1/36; A61K 31/437; C07K 16/24

(52) U.S. Cl. CPC A61N 1/36082; A61K 31/437; C07K 16/243

(57) ABSTRACT

A method for increasing phase locking of neurons to gamma oscillations in at least one brain region of a subject for treating Alzheimer's disease in the subject in need thereof includes administering an inhibitor including a colony-stimulating factor-1 receptor (CSF1R) inhibitor or a colony-stimulating factor-1 (CSF1) inhibitor to the subject. The method also includes administering a stimulus to the subject having a frequency from about 20 Hz to about 60 Hz.

### CROSS-REFERENCE TO RELATED APPLICATIONS

This application claims priority to U.S. Provisional Application No. 63/288,046 titled “SYSTEMS, DEVICES, AND METHODS FOR ENHANCING THE NEUROPROTECTIVE EFFECTS OF NON-INVASIVE GAMMA STIMULATION WITH PHARMACOLOGICAL AGENTS”, filed Dec. 10, 2021, and to U.S. Provisional Application No. 63/381,855 titled “EFFECT OF DEPLETED MICROGLIA ON AMYLOID PLAQUE FORMATION IN APOE4 MOUSE MODELS”, filed Nov. 1, 2022, the entire disclosures of which are incorporated herein by reference.

### BACKGROUND

Alzheimer's disease (AD) is a debilitating and highly prevalent brain disorder that accounts for 60-80% of dementia cases, with more than 20% of people over age 75 being affected. There is a pressing need to both understand the mechanisms of AD and to find treatments for AD. Without being limited by theory, it is understood that some microglia, operating in a benign manner in normal brains, become (undesirably) activated during AD, which can lead to neuroinflammation and generally contribute to disease pathogenesis. It has been observed that pharmacological reduction of microglia by colony-stimulating factor receptor-1 (CSF1R) inhibition produces protective effects in mouse models of AD, but that the population of microglia is only partially reduced. As a result, pathology is only partially, and usually insufficiently, affected. It is also unclear to what extent microglia are activated and/or otherwise compromised during AD.

Neural oscillations, particularly gamma oscillations, which reflect interactions between groups of neurons, are impaired in AD. Recent studies have used visual, haptic, and/or auditory stimulation to noninvasively induced neural oscillations around gamma frequencies in multiple AD mouse models. Further, significant reductions in amyloid-beta (Aβ) peptides and amyloid plaque levels as well as effects on microglia, astrocytes, and the brain vasculature have been observed. Additionally, it has been found that chronic stimulation (i.e., for longer durations) in these mouse models reduced neuroinflammation, phosphorylation of tau protein, neurodegeneration, and loss of synapses while improving cognitive performance. Accordingly, modulation of the functioning of microglia may be implicated by these observations. Further, some studies have shown that the presence of the Apolipoprotein E4 (APOE4) allele results in the greatest risk of AD to a subject, since APOE4 carriers tend to accumulate amyloid earlier than non-carriers, and also exhibit a relatively higher microglia association with amyloid plaque levels.

### SUMMARY

The inventors have accordingly appreciated the limited efficacy of pharmacological reduction of microglia and have therefore recognized an unmet need to determine whether overlapping administration of pharmacological agents (e.g., inflammatory drugs, such as CSF1R inhibitors) and visual and/or auditory stimulation has synergistic effects, and in particular, whether they reduce pathology associated with neurodegenerative disease, or other pathological conditions, in the brain of a subject, while improving neuronal networks and cognitive function, among others.

In view of the foregoing, the inventive concepts disclosed herein relate to the inventors' investigation into the use of pharmacological agents together with non-invasive audio, visual and/or haptic stimulation (e.g., in the gamma regime) to reduce pathology in the brain. As discussed in further detail herein, the inventors have observed that administration of inhibitors such as Plx3397 coupled with administration of non-invasive gamma stimulation can result in significant reduction in inflammatory markers, increased expression of extracellular matrix reorganization genes in microglia, and neurons that are much more strongly phase locked with gamma oscillations. Without being limited by any theory in particular, the inventors have conceived of and demonstrated a process in which starting treatment with a CSF1R inhibitor reduces microglia and microglia-mediated inflammation, including reducing loss of synaptic density. Subsequently, the application of visual and/or auditory gamma stimulation can then strengthen the preserved synapses, among other benefits.

Inventors also observed that, in APOE4 carriers in particular, reduction of microglia via CSF1R inhibitors alone may not be sufficient to clear amyloid plaques, but may nevertheless improve amyloid clearance by visual and/or auditory stimulation as described herein.

Accordingly, some aspects are directed to a method for increasing phase locking of neurons to gamma oscillations in at least one brain region of a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method also includes administering a stimulus to the subject having a frequency from about 20 Hz to about 60 Hz.

Some aspects are directed to a method for increasing phase locking of neurons to gamma oscillations in at least one brain region of a subject. The subject has been administered an inhibitor including a colony-stimulating factor-1 receptor (CSF1R) inhibitor or a colony-stimulating factor-1 (CSF1) inhibitor. The method includes administering a stimulus to the subject having a frequency from about 20 Hz to about 60 Hz.

Some aspects are directed to a method that includes providing a device that administers a stimulus to a subject during use of the device, the subject having been administered an inhibitor including a colony-stimulating factor-1 receptor (CSF1R) inhibitor or a colony-stimulating factor-1 (CSF1) inhibitor. The stimulus has a frequency of from about 20 Hz to about 60 Hz.

Some aspects are directed to a method for treating Alzheimer's disease in a subject in need thereof, the method including administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for reducing a number of microglia in at least one brain region of a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method also includes non-invasively administering a stimulus to the subject having a frequency from about 20 Hz to about 60 Hz.

Some aspects are directed to a method for increasing synaptic density in at least one brain region of a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for increasing neuronal density in at least one brain region of a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for reducing neuroinflammation in at least one brain region of a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for reducing expression of genes associated with protein synthesis in microglia in a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for increasing expression of genes associated with clearing of low-density lipoprotein in a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for increasing expression of genes associated with vesicle organization in a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for increasing the perineuronal net of neurons in at least one brain region of a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for increasing expression of genes associated with extracellular matrix organization in a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for improving memory in a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for improving cognitive function in a subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for increasing phase locking of neurons to theta oscillations in at least one brain region of a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for increasing myelination in at least one brain region of a subject for treating Alzheimer's disease in the subject in need thereof. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

Some aspects are directed to a method for reducing microglia in at least one brain region of a subject for treating Alzheimer's disease in the subject in need thereof, the subject having at least one Apolipoprotein E4 (APOE4) allele. The method includes administering an inhibitor including a colony-stimulating factor-1 receptor (CSF1R) inhibitor or a colony-stimulating factor-1 (CSF1) inhibitor to the subject. The method further includes non-invasively administering a stimulus to the subject having a frequency from about 20 Hz to about 60 Hz.

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIGS. 1A-1D show Plx3397 and/or GENUS treatments impact microglia density and morphology in the visual cortex in 5×FAD mice. ANOVA with post-hoc comparisons, *, **, ***, **** and ns indicate P<0.05, P<0.01, P<0.001, P<0.0001 and not significant, respectively

FIG. 1A shows an experimental outline to reduce microglia and administer GENUS.

FIG. 1B shows example confocal images. Scale bar=20 μm.

FIG. 1C shows IBA1+ cell density expressed as % no treatment control.

FIG. 1D shows volume of Iba1+ cells.

FIGS. 2A-2B show that Plx3397 and/or GENUS treatments improve synaptic density in the visual cortex in 5×FAD mice. ANOVA with post-hoc comparisons, *, **, ***, **** and ns indicate P<0.05, P<0.01, P<0.001, P<0.0001 and not significant, respectively. N=8-9 mice per group.

FIG. 2A shows example confocal images. Scale bar=50 μm.

FIG. 2B shows vGAT synaptic puncta expressed as % of no treatment control.

FIGS. 3A-3B show that Plx3397 and/or GENUS treatments improve synaptophysin in the visual cortex in 5×FAD mice. ANOVA with posthoc comparisons, *, **, *** and ns indicate P<0.05, P<0.01, P<0.001 and not significant, respectively. N=8-9 mice per group.

FIG. 3A shows uncropped original immunoblots.

FIG. 3B shows synaptophysin signal intensity expressed as % of no treatment control.

FIGS. 4A-4B show that Plx3397+GENUS treatments improve neuronal density in the visual cortex in 5×FAD mice. ANOVA with posthoc comparisons, *, and ns indicate P<0.05 and not significant, respectively. N=6-7 mice per group.

FIG. 4A shows example confocal images.

FIG. 4B shows NeuN density expressed as % of no treatment control.

FIGS. 5A-5D show that Plx3397 and/or GENUS treatments reduce inflammatory markers in the visual cortex in 5×FAD mice. ANOVA with posthoc comparisons, *, **, ***, **** and ns indicate P<0.05, P<0.01, P<0.001, P<0.0001, and not significant, respectively. N=8-9 mice per group.

FIG. 5A shows example confocal images of C1q. Scale bar=100 μm.

FIG. 5B shows C1q signal intensity expressed as % of no treatment control.

FIG. 5C shows example confocal images of MHC2. Scale bar=50 μm.

FIG. 5D shows MHC2 signal intensity expressed as % of no treatment control.

FIGS. 6A-6D show that Plx3397 and/or GENUS treatments improve synaptic marker while reducing inflammatory marker in the hippocampus in 5×FAD mice. ANOVA with posthoc comparisons, *, **, ***, **** and ns indicate P<0.05, P<0.01, P<0.001, P<0.0001, and not significant, respectively. N=8-9 mice per group.

FIG. 6A shows example confocal images of IBA1, vGAT, and C1q.

FIG. 6B shows microglia density expressed as % of no treatment control.

FIG. 6C shows vGAT density expressed as % of no treatment control.

FIG. 6D shows C1q signal intensity expressed as % of no treatment control.

FIGS. 7A-7H show that Plx3397 and/or GENUS treatments improve synaptic marker while reducing inflammatory marker in the CK-p25 mice. ANOVA with post-hoc comparisons, *, **, ***, **** and ns indicate P<0.05, P<0.01, P<0.001, P<0.0001, and not significant, respectively. N=7-11 mice/group.

FIG. 7A shows an experiment outline.

FIG. 7B shows example confocal images of IBA1.

FIG. 7C shows IBA1+ cell density expressed as % no treatment control.

FIG. 7D shows volume of Iba1+ cells.

FIG. 7E shows uncropped original synaptophysin immunoblots.

FIG. 7F shows synaptophysin signal intensity expressed as % of no treatment control.

FIG. 7G shows C1q signal intensity expressed as % of no treatment control.

FIG. 7H shows γH2Ax positive neurons expressed as % of no treatment control.

FIGS. 8A-8D show single-cell gene expression analysis after Plx3397 and/or GENUS treatments in the 5×FAD mice.

FIG. 8A shows a UMAP showing clusters of cells based on the gene expression patterns.

FIG. 8B shows cells in each cluster are represented from all groups as seen in the color-coded UMAP.

FIG. 8C shows microglia clusters that were identified based on the expression levels of marker genes shown to the top of UMAPs.

FIG. 8D shows oligodendrocyte clusters that were identified based on the expression levels of marker genes shown to the top of UMAPs.

FIGS. 9A-9C show that Plx3397 and/or GENUS treatments impact a unique set of genes in microglia in 5×FAD mice.

FIG. 9A shows the overlap of number of genes significantly upregulated in Plx3397, GENUS or Plx3397+GENUS treated mice (cut off of log 2 fold with ±0.3 difference and a P value of less than 0.01) compared to control-treated 5×FAD mice in microglia cluster.

FIG. 9B shows that Plx3397+GENUS treatment significantly increased gene expression compared to either treatment alone.

FIG. 9C shows commonly upregulated genes are listed, and the gene enrichment biological process analyses is shown to the right. Overlap of a number of genes significantly downregulated after these treatments. Commonly downregulated genes are listed, and the gene ontology terms are shown to the right.

FIG. 10A-10C show that the combined administration of CSF1 inhibitor and GENUS induces gene expression changes in microglia.

FIG. 10A shows a UMAP showing microglia specific cluster (left panel). The middle and right panels show gene ontology terms (functions of groups of genes) of up (middle) and down-regulated (right) genes after Plx3397 and/or GENUS treatment.

FIG. 10B shows a UMAP showing sub-clusters of microglia (Cluster numbers 0, 2, 3, 7, 9 10, 11, & 13) (left panel). The middle and right panels show gene ontology terms of commonly or uniquely up (middle) and down-regulated (right) genes after Plx3397+GENUS treatment.

FIG. 10C shows volcano plots show up (red data points) and down-regulated (blue data points) genes in representative sub-cluster of microglia. Genes related to increased myelination and reduced MHC-class 2 antigen presentations are highlighted.

FIGS. 11A-11D show that the combined administration of CSF1 inhibitor and GENUS induces gene expression changes in oligodendrocytes. ANOVA with posthoc comparisons, *, and ns indicate P<0.05, and not significant, respectively. N=6-7 mice per group.

FIG. 11A shows a UMAP showing clusters of cells based on gene expression patterns.

FIG. 11B shows a UMAP showing oligodendrocytes specific marker genes. They are enriched in cluster 1, thus cluster 1 cells are oligodendrocytes.

FIG. 11C shows gene ontology terms (functions of groups of genes) of up (top) and down-regulated (bottom) genes after Plx3397 and/or GENUS treatment.

FIG. 11D shows a volcano plot showing up (red data points) and down-regulated (blue data points) genes in oligodendrocytes cluster. Genes related to increased myelination and reduced MHC-class 2 antigen presentations and complement pathways are highlighted.

FIG. 12 shows that Plx3397 and/or GENUS treatments improve myelination protein plasmolipin in the visual cortex in 5×FAD mice. Top: Uncropped original immunoblots. Bottom. Plasmolipin signal intensity expressed as % of no treatment control. ANOVA with posthoc comparisons, *, and ns indicate P<0.05, and not significant, respectively. N=6-7 mice per group.

FIG. 13A-13D show that the combined administration of CSF1 inhibitor and GENUS induces gene expression changes in neurons.

FIG. 13A shows a UMAP representation of clusters of cells based on gene expression patterns after single-nucleus RNA-sequencing.

FIG. 13B shows gene ontology term showing upregulated genes in interneuron cluster.

FIG. 13C shows a table showing sub-cellular enrichment of upregulated genes in interneuron cluster.

FIG. 13D shows a volcano plot showing up (red data points) and down-regulated (blue data points) genes in interneuron cluster. Genes related to increased myelination and synaptic transmission are highlighted.

FIG. 14 shows that the combined administration of CSF1 inhibitor and GENUS induces synaptic gene expressions. Volcano plots show up (red data points) and down-regulated (blue data points) genes in all-neurons and astrocytes clusters. Upregulated genes related to synapses is highlighted.

FIGS. 15A-15B show 40 Hz entrainment in the 5×FAD mice treated with Plx3397. ANOVA with posthoc comparisons; ***, and ns indicate P<0.001, and not significant, respectively. N=4 mice/group.

FIG. 15A shows time-resolved spectrogram showing LFP power before, after, and during 40 Hz stimulation in the visual cortex in 5×FAD with or without Plx3397 treatment.

FIG. 15B shows grouped LFP power spectra showing a significant increase in gamma power during gamma stimulation.

FIG. 16A-16D show that Plx3397 and/or GENUS treatments enhance the gamma phase of neurons in 5×FAD mice. ANOVA with posthoc comparisons; *, **, and ns indicate P<0.05, P<0.01, and not significant, respectively.

FIG. 16A shows an example waveform of the action potential of putative excitatory neurons and interneurons.

FIG. 16B shows three representative interneurons showing 40 Hz entrainment with harmonic or subharmonic response in Plx3397+GENUS treated 5×FAD mice.

FIG. 16C shows a polar plot showing spike probability across LFP gamma phase.

FIG. 16D shows gamma phase locking of excitatory neurons and interneurons in all groups.

FIGS. 17A-17C shows that Plx3397 and/or GENUS treatments enhance perineuronal net in 5×FAD mice. ANOVA with posthoc comparisons; *, **, and ns indicate P<0.05, P<0.01, and not significant, respectively.

FIG. 17A show example confocal images of WFA. Scale bar=100 μm.

FIG. 17B shows WFA signal intensity expressed as % of no treatment control

FIG. 17C shows WFA surface volume expressed as % of no treatment control.

FIGS. 18A-18C show that Plx3397 and/or GENUS treatments enhance synaptic input within the perineuronal net in 5×FAD mice. ANOVA with posthoc comparisons; *, **, and ns indicate P<0.05, P<0.01, and not significant, respectively.

FIG. 18A shows 3D rendered example confocal images of WFA and presynaptic marker vGlut1. Scale bar=20 μm (top) and 3 μm (bottom).

FIG. 18B shows vGLUT1 puncta in the visual cortex expressed as % of no treatment control.

FIG. 18C shows vGLUT1 puncta within WFA surface.

FIGS. 19A-19I show that Plx3397 and/or GENUS treatments improve object recognition memory in multiple mouse models of neurodegeneration. ANOVA with post-hoc comparisons, *, **, ***, **** and ns indicate P<0.05, P<0.01, P<0.001, P<0.0001, and not significant, respectively.

FIG. 19A shows a schematic of test in 5×FAD, and mice occupancy heatmaps.

FIG. 19B shows time spent in the center during OF.

FIG. 19C shows a schematic of NOR habituation and the corresponding mice occupancy heatmaps.

FIG. 19D shows novelty index during NOR habituation in 5×FAD mice.

FIG. 19E shows a schematic of NOR test and the corresponding mice occupancy heatmaps.

FIG. 19F shows novelty index during NOR test in 5×FAD mice.

FIG. 19G shows time spent in center during OF test in CK-p25 mice

FIG. 19H shows total distance traveled during OF test in CK-p25 mice.

FIG. 19I shows novelty index during NOR test in CK-p25 mice.

FIGS. 20A-20B show that GENUS reduced amyloid levels in the cortex compared to no stimulation control mice, whereas levetiracetam co-administration occluded the effect of 40 Hz. ANOVA with post-hoc comparisons, *, **, and ns indicate P<0.05, P<0.01, and not significant, respectively.

FIG. 20A shows example confocal images of amyloid. Scale bar=200 μm.

FIG. 20B shows amyloid signal intensity expressed as % of no treatment control.

FIGS. 21A-21Q show that chronic Plx3397 treatment reduces the percentage of gamma and theta phase locking of neurons in 5×FAD mice. Numbers in charts n and p represent neurons out of total neurons significantly (p<0.05) phase-locked to gamma and theta oscillations in each comparison.

FIG. 21A shows an experiment outline. 5×FAD mice were administered with regular diet or diet containing Plx3397 for 50 days.

FIG. 21B shows in vivo electrophysiological recording configuration. Linear probes were implanted in the visual cortex. Example images show linear probe recording locations (hoechst3352 stain).

FIG. 21C shows example confocal images showing IBA1 and GFAP signals in control and Plx3397 treated 5×FAD mice. Scale bar=50 μm.

FIG. 21D shows that Plx3397 reduced IBA1+ but not GFAP+ cells. 2 way ANOVA, treatment x cells interaction, F (1, 6)=36.10, p=0.0010. n=4 mice/group.

FIG. 21E shows power spectra of LFP in control and Plx3397 treated 5×FAD mice. 2 W RM ANOVA, treatment x frequency interaction, F (201, 1206)=2.519, p<0.0001. There was no group difference between control and Plx3397 treated 5×FAD mice (2 W RM ANOVA, F (1, 6)=3.425, p=0.0993). au=arbitrary units.

FIG. 21F shows plots showing unprocessed raw LFP traces and the corresponding time-resolved power spectra from 5×FAD without or with Plx3397 administration. Representative time-resolved power spectra from layer 4 LFP (top), and LFP power spectra organized according to cortical depth (middle & bottom) from plx3397 treated 5×FAD mice.

FIG. 21G shows L2/3, L4, L5, & L6 that indicate cortical layers 2/3, 4, 5, & 6, respectively. Arrow marks show the distinct theta-burst and gamma states.

FIG. 21H shows current source density (CSD) plots of theta-burst (3-12 Hz) from Plx3397 treated 5×FAD mice. Scale bar=200 ms, 200 μV.

FIG. 21I shows theta-burst CSD profile in each L2/3, L4, L5, & L6 cortical layer. Time 0 represents theta-burst onset, and each line represents a mouse (N=4 mice).

FIG. 21J shows duration and ratio of peak-to-trough (P-T) of single units from Plx3397 5×FAD mice. Inset shows spike waveforms of representative E-neuron and I-neuron.

FIG. 21K shows mean spike rate of E-neurons (unpaired t-test, t=0.9159, p=0.3612) and I-neurons (t-test, t=1.273, p=0.2077) did not differ between control and Plx3397 treated 5×FAD mice.

FIG. 21L shows single unit raster plot showing spiking during pre-, post-, and theta-bursts in Plx3397 treated 5×FAD mice.

FIG. 21M shows mean spike rate of neurons in each cortical layer at the onset (0 to −200 ms) of theta-burst and ±800 ms (gamma states).

FIG. 21N shows n, o. plots showing the percentage of E-neurons (gray) and I-neurons (blue) phase-locked to gamma oscillations in Plx3397 and control 5×FAD mice.

FIG. 21M shows plots showing the strength of phase locking in Plx3397 and control 5×FAD mice.

FIG. 21P shows plots showing the % of E-neurons and I-neurons phase-locked to theta-bursts in Plx3397 and control 5×FAD mice.

FIG. 21Q shows plots showing the strength of phase locking in Plx3397 and control 5×FAD mice.

FIGS. 22A-22G show that chronic Plx3397 treatment modifies synaptic and extracellular matrix proteins in 5×FAD mice. Scale bar=100 or 50 m as indicated. N=5-6 mice per group, au=arbitrary units.

FIG. 22A shows example confocal images showing D54D2 amyloid, myelin basic protein (MBP), complementary molecule C1q, synaptophysin, Wisteria floribunda agglutinin (WFA), and aggrecan co-stained with parvalbumin (PV) in the control and Plx3397 administered 5×FAD mice.

FIG. 22B is a graph showing that Plx3397 did not affect amyloid (unpaired t-test, t=1.544).

FIG. 22C is a graph showing that Plx3397 did not affect MBP levels (t=0.4076).

FIG. 22D is a graph showing that Plx3397 reduced C1q levels (t=2.699, p=0.0244).

FIG. 22E is a graph showing that Plx3397 increased synaptophysin (t=2.273).

FIG. 22F is a graph showing that Plx3397 increased WFA signals (t=3.774).

FIG. 22G is a graph showing that Plx3397 reduced aggrecan intensity within the soma of PV interneurons (nested t-test, t=2.298, f=5.280).

FIGS. 23A-23I show sensory evoked gamma oscillations improve neural function in Plx3397 treated 5×FAD mice.

FIG. 23A shows spectral power of LFP during baseline with 4 Hz (a) stimulation.au=arbitrary units, and N=4 mice/group.

FIG. 23B shows spectral power of LFP during baseline with 40 Hz (b) stimulation. au=arbitrary units, and N=4 mice/group.

FIG. 23C shows representative LFP trace during 4 Hz entrainment (top) and LFP waveforms as a function of 4 Hz stimulus (bottom).

FIG. 23D shows representative LFP trace during 40 Hz entrainment (top), and LFP waveforms as a function of 40 Hz stimulus (bottom left). Aberrant theta-burst was significantly reduced during acute 40 Hz entrainment (bottom right, Mann-Whitney U=1304, p=0.0001).

FIG. 23E shows three simultaneously recorded I-neurons from L2/3, L4 and L6 showed 40 Hz entrainment. Polar plots (right) show spike probability along LFP theta and gamma phases during baseline and 40 Hz entrainment. Rayleigh statistics and mean resultant length (MRL) indices indicate whether neuronal spiking is phase-locked to LFP and the phase-locking strength, respectively.

FIG. 23F shows a higher percentage of both E-neurons and I-neurons were phase-locked to gamma during 40 Hz gamma entrainment.

FIG. 23G shows gamma phase-locking strength of E-neurons (2 W ANOVA, F (1, 97)=10.21, p=0.0019) and I-neurons (2 W RM ANOVA, F (1, 43)=14.50, p=0.0004) were higher during 40 Hz entrainment. Data are single units.

FIG. 23H shows the experiment outline (left). LFP power spectrogram before, during, and after 40 Hz stimulation in GENUS (top) and Plx3397+GENUS (middle) treated 5×FAD mice. The line plot (bottom) shows the gamma power change during 40 Hz entrainment.

FIG. 23I shows twenty simultaneously recorded single units were organized according to cortical layers in Plx3397+GENUS treated mice. Spike waveforms of isolated units and power spectral density of units are shown.

FIG. 24A-24M show chronic gamma entrainment enhances MEF2C in Plx3397 treated 5×FAD mice.

FIG. 24A shows an experiment outline to administer CSF1R inhibitor and/or GENUS in 5×FAD mice.

FIG. 24B shows confocal images showing IBA1 immunosignals. Scale bar=50 m.

FIG. 24C shows IBA1+ microglial numbers (AONVA, F (3, 29)=49.37, p=0.0001) expressed as % no treatment control.

FIG. 24D shows % area covered by the IBA1+ optical signal (ANOVA, F (3, 29)=25.12, p=0.0001).

FIG. 24E shows a UMAP visualization of snRNA-seq from visual cortex from 11-month-old 5×FAD mice colored by cell type.

FIG. 24F shows a dot plot demonstrating scaled gene expression for cluster markers for each cell type.

FIG. 24G shows a Venn diagram showing overlap of differentially expressed genes. Genes related to the molecular pathway (trans-synaptic signaling) and mouse phenotype (abnormal CNS synaptic transmission) were rescued after GENUS in Plx3397 treated 5×FAD mice. p refers to false discovery rate corrected p-value.

FIG. 24H shows a Venn diagram of differentially expressed genes. GENUS rescued head and brain development genes.

FIG. 24I shows the top 10 upregulated gene ontology biological functions for excitatory neurons & interneurons from DEGs in Plx3397+40 Hz group compared to Plx3397 alone.

FIG. 24J shows the top 10 downregulated gene ontology biological functions for excitatory neurons & interneurons from DEGs in Plx3397+40 Hz group compared to Plx3397 alone.

FIG. 24K shows the number of DEGs in Plx3397+GENUS compared to Plx3397 alone from snRNA-seq. Volcano plots of differentially expressed genes in excitatory neurons and interneurons. Red dots represent upregulated transcripts, while blue dots represent downregulated transcripts in Plx3397+GENUS compared to control 5×FAD mice. y-axes represent adjusted log 2 p-value for cluster changes.

FIG. 24L shows representative confocal images of MEF2C. Scale bar=50 μm.

FIG. 24M shows quantification showing MEF2C (ANOVA, F (3, 29)=5.863, p=0.0029) expressed as % of no treatment control.

FIGS. 25A-25M show chronic gamma entrainment improves synaptic input within PNN and novel object recognition in Plx3397 treated 5×FAD mice.

FIG. 25A shows western blots of synaptophysin (syn), vGLUT1, MBP, and beta-actin.

FIG. 25B shows summary graphs showing expression levels of synaptophysin (ANOVA, F (3, 28)=4.230, p=0.0138).

FIG. 25C shows summary graphs showing expression levels of vGLUT1 (ANOVA, F (3, 28)=4.371, p=0.0121).

FIG. 25D shows representative confocal images of vGAT synaptic puncta (scale bar=20 μm), WFA (100 μm), and 3D rendered example confocal images of WFA and synaptic marker vGLUT1 (20 μm). Co-labeled WFA, MBP, and PV are shown (20 m; inset 10 μm). Example confocal images of Neun (50 μm).

FIG. 25E shows summary graphs showing vGAT synaptic puncta as % of no treatment control (ANOVA, F (3, 29)=8.831, p=0.0003),

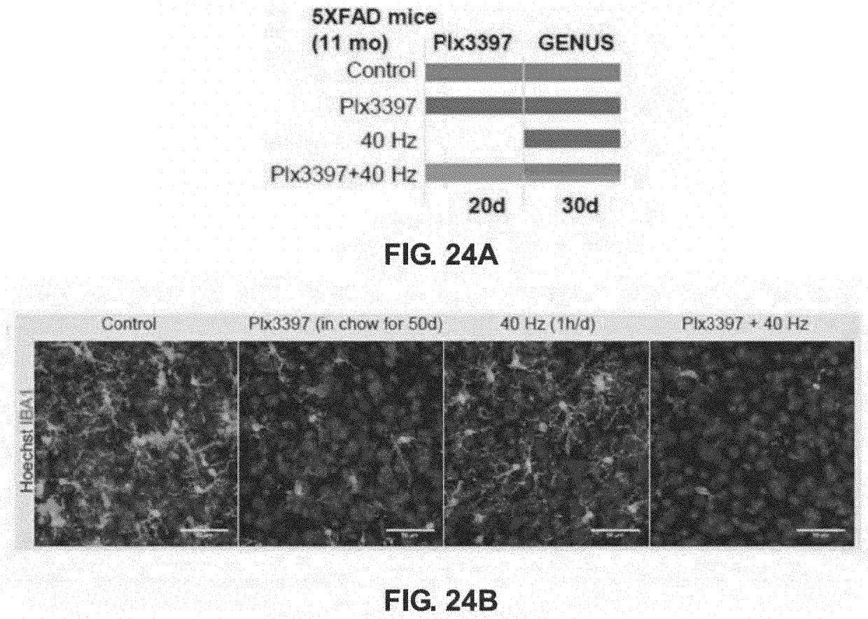

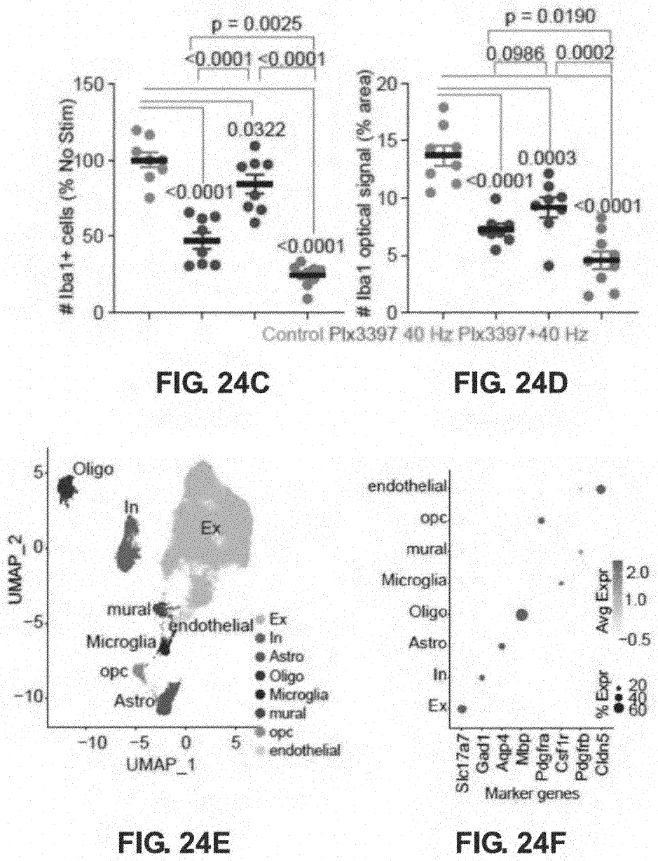

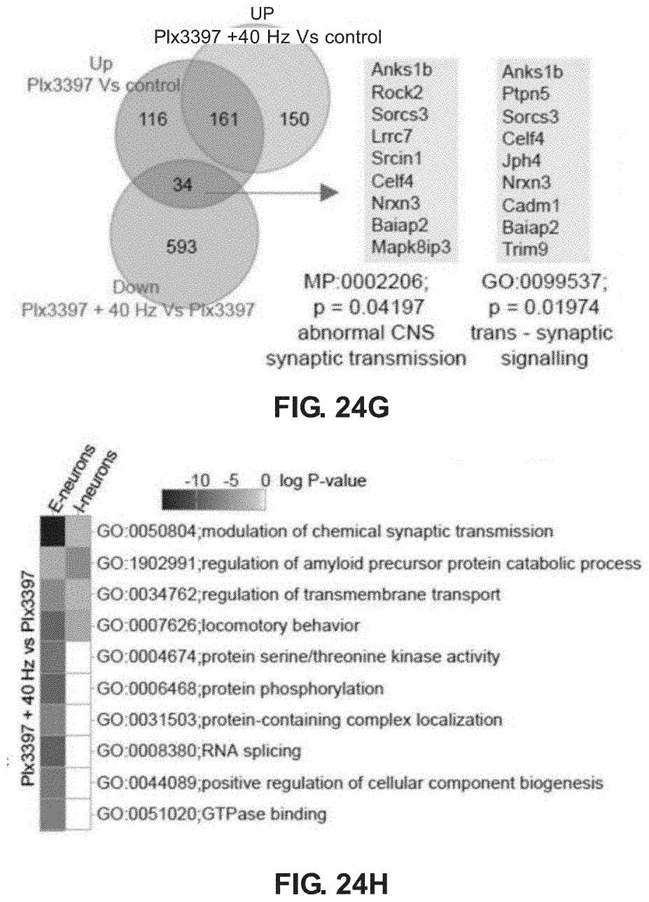

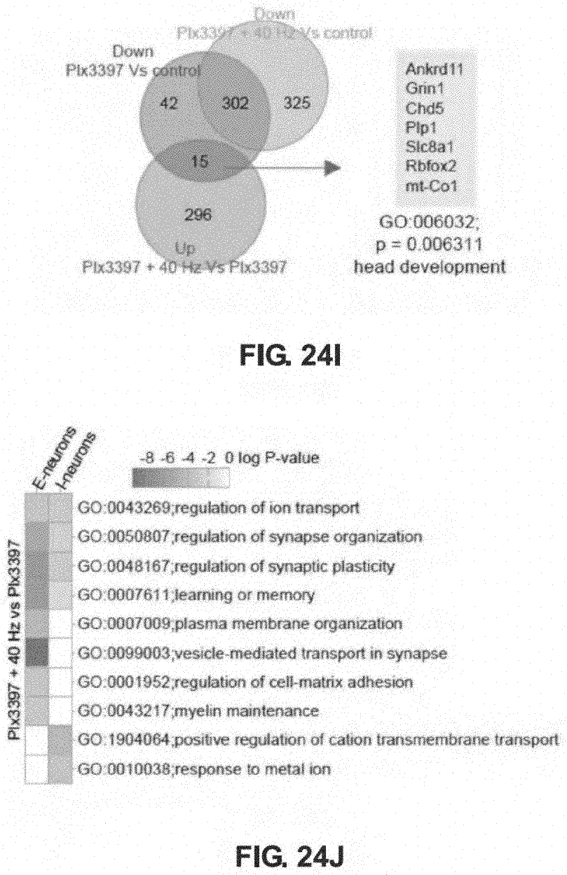

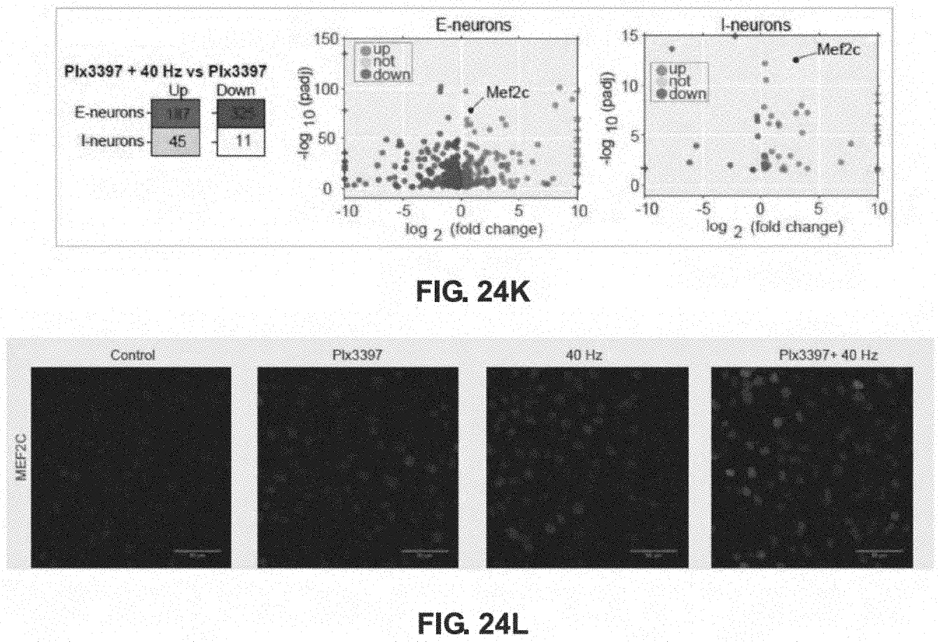

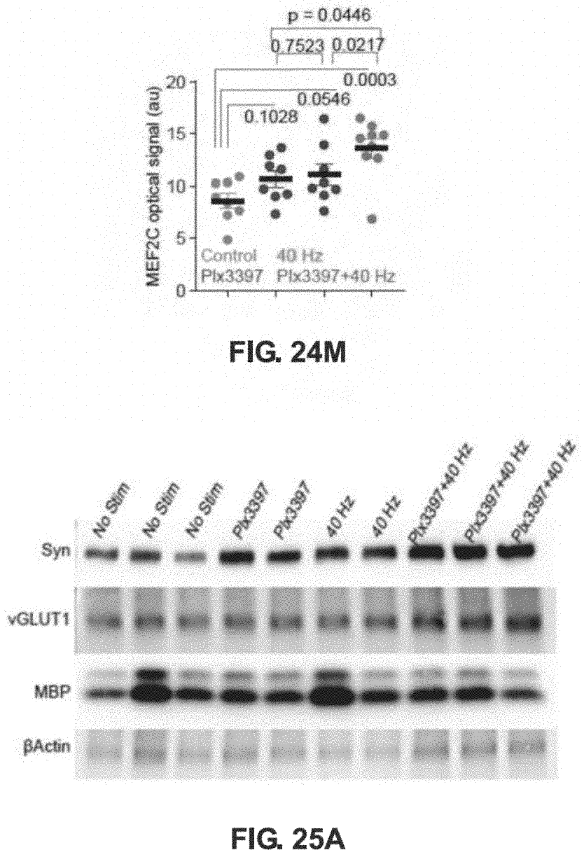

FIG. 24F shows summary graphs showing WFA optical signal as % of no treatment control (ANOVA, F (3, 29)=4.307, p=0.0125).

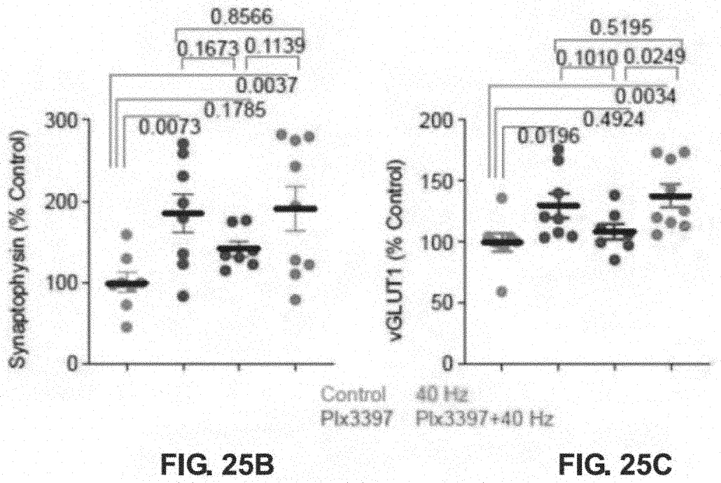

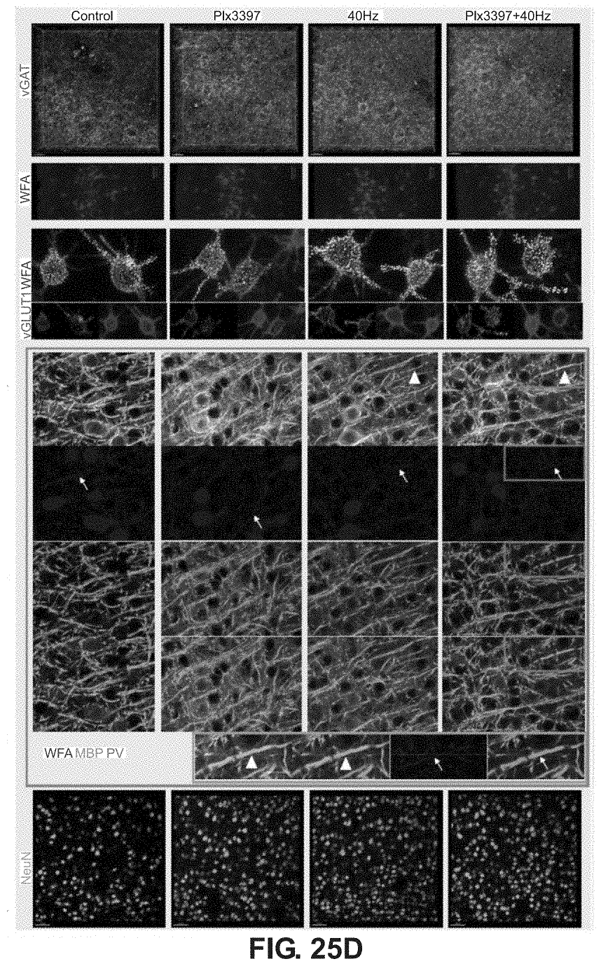

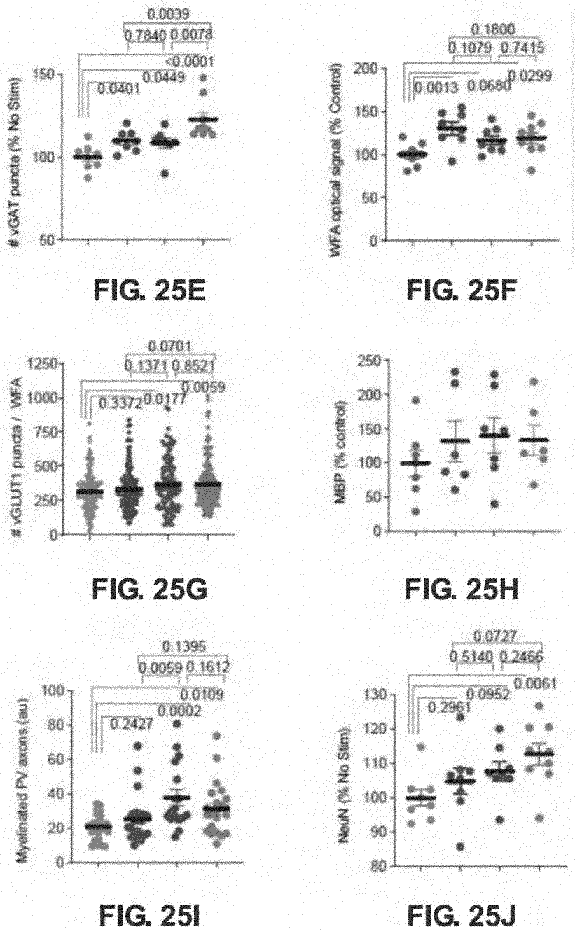

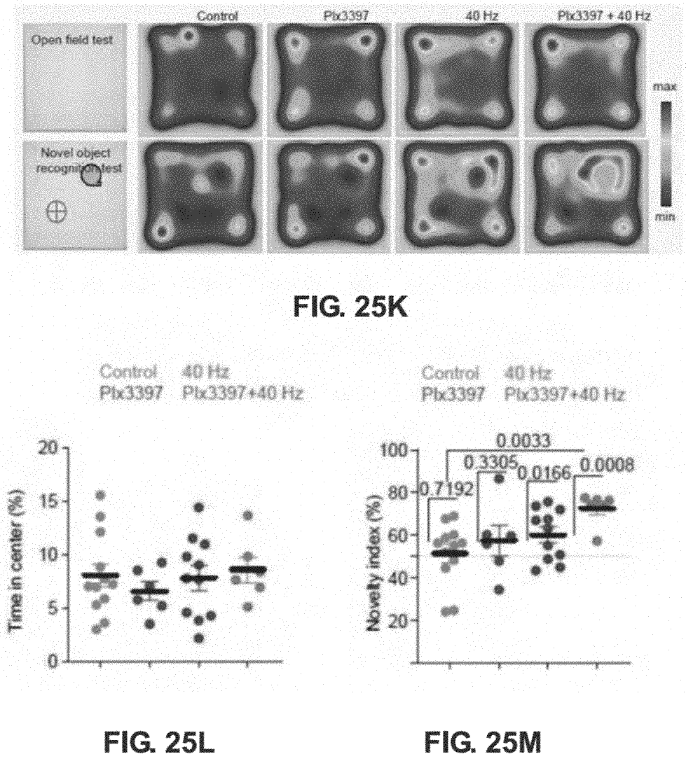

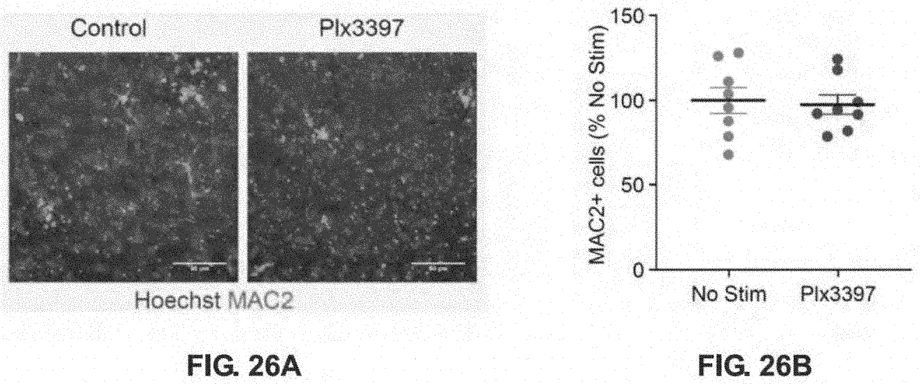

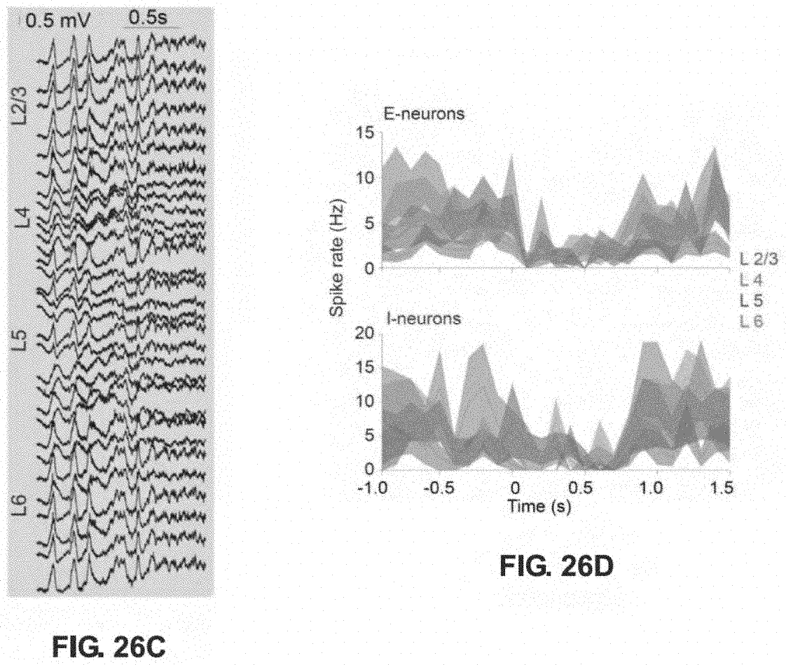

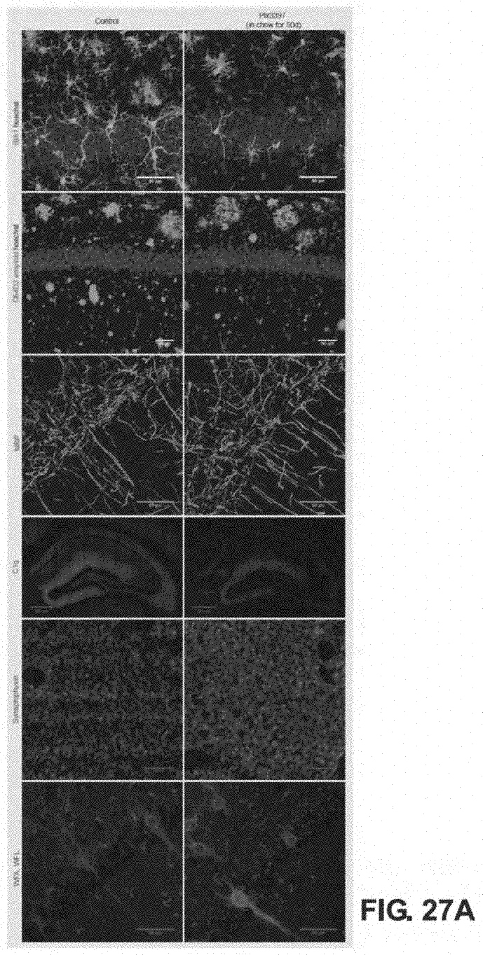

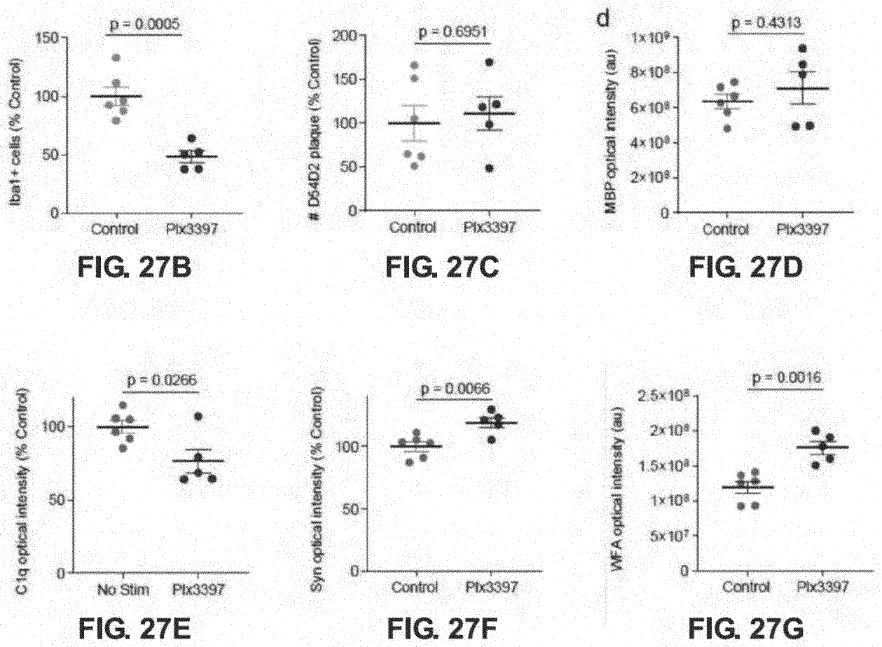

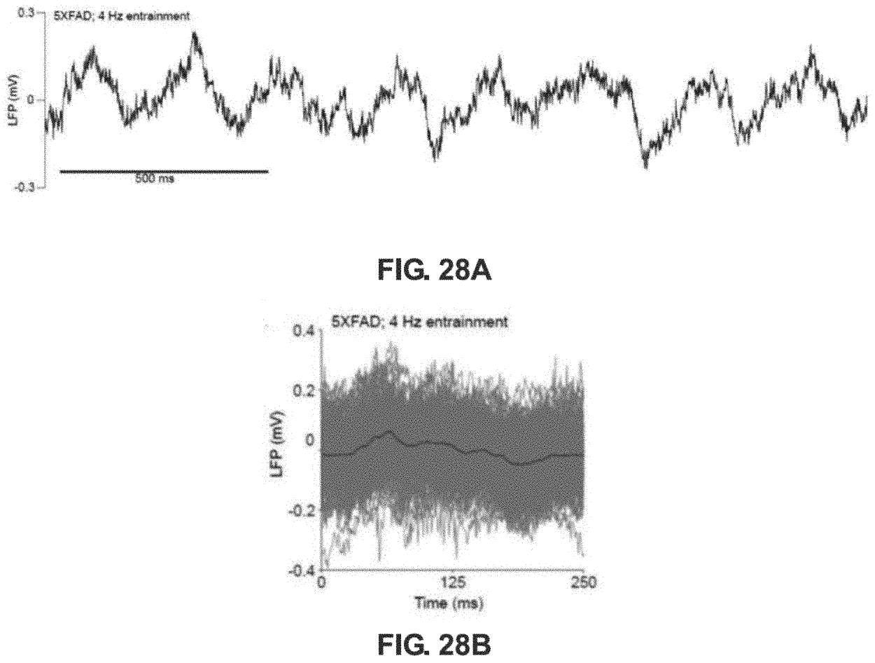

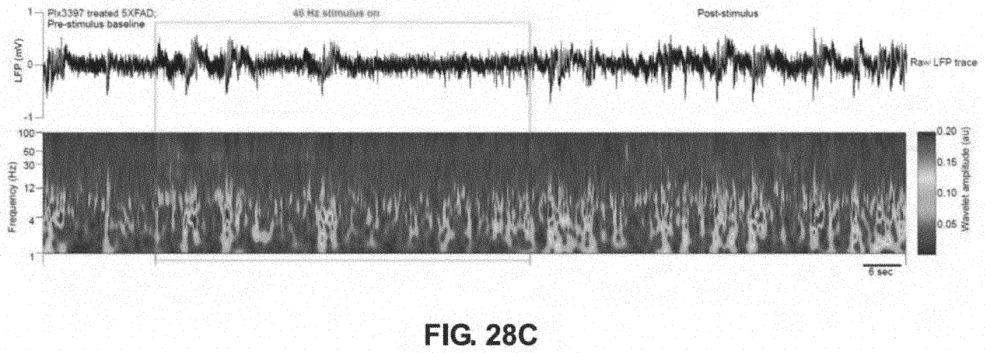

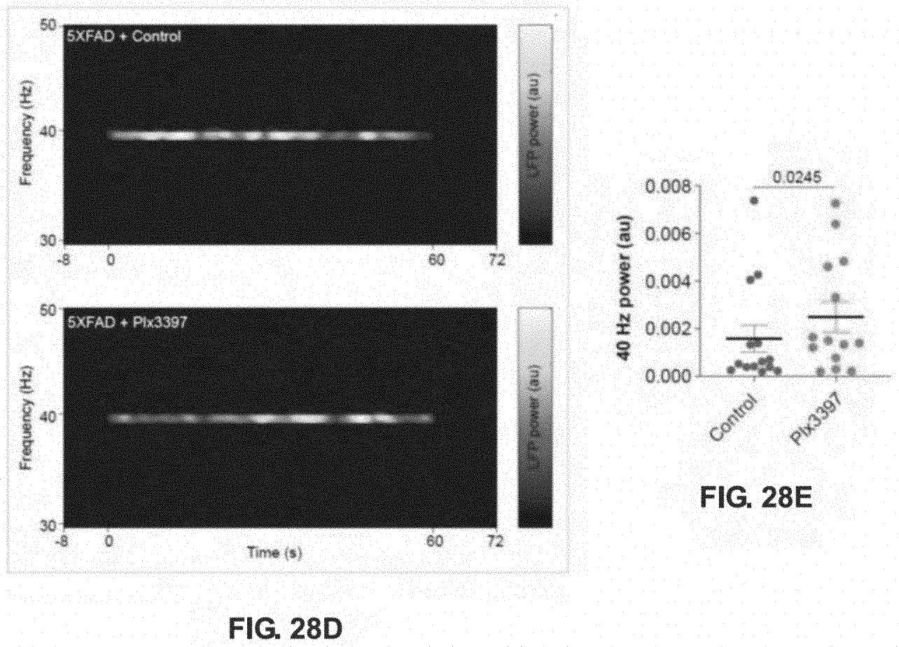

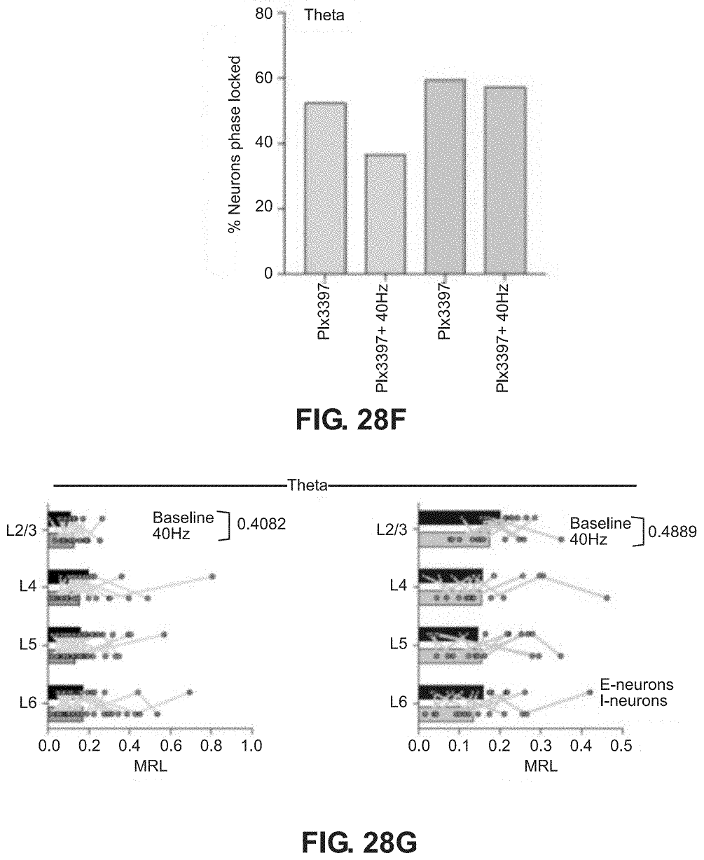

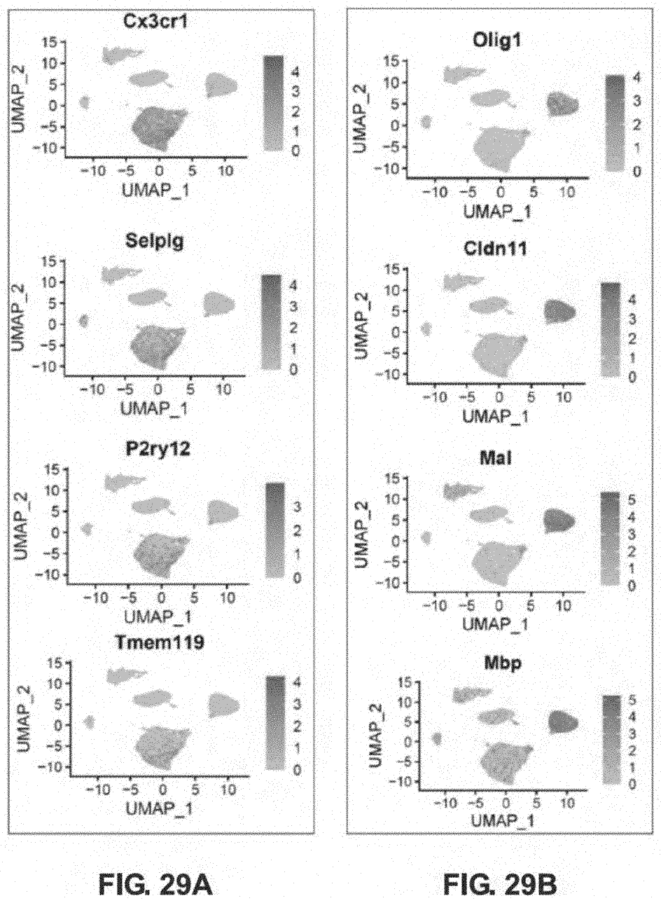

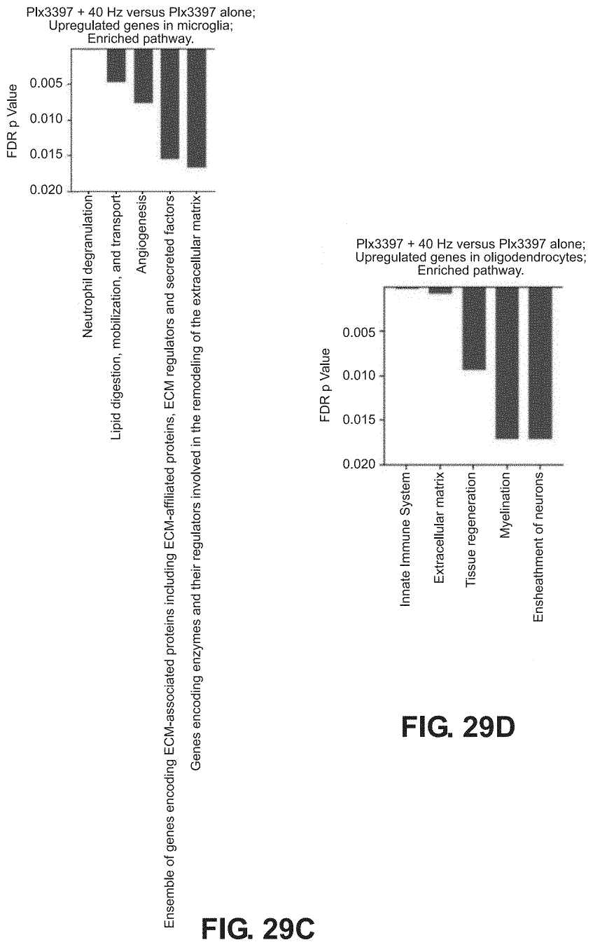

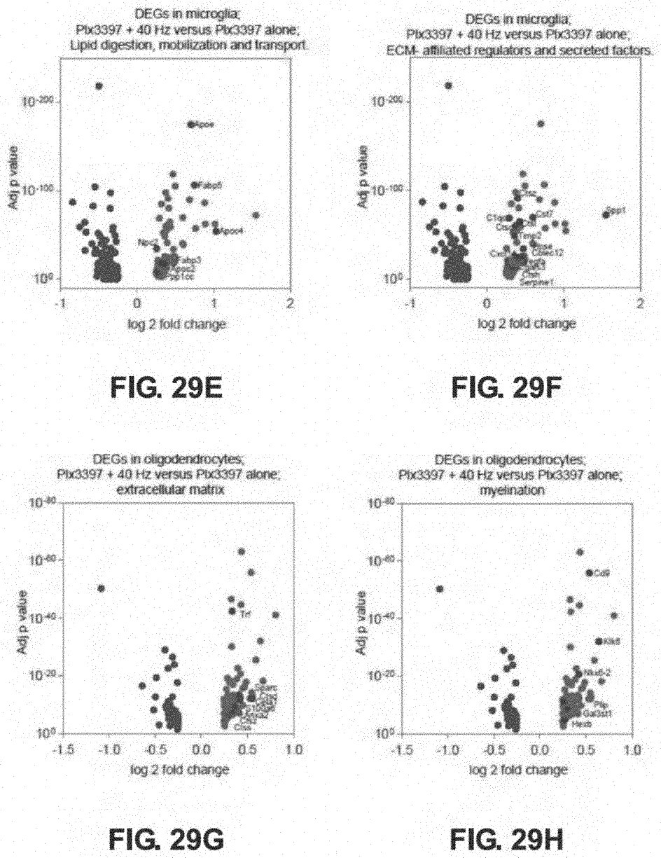

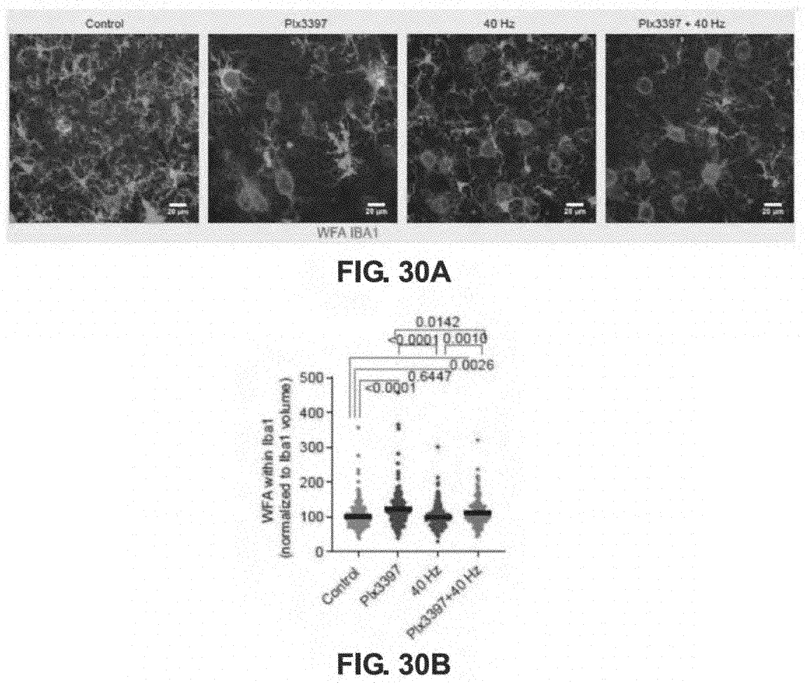

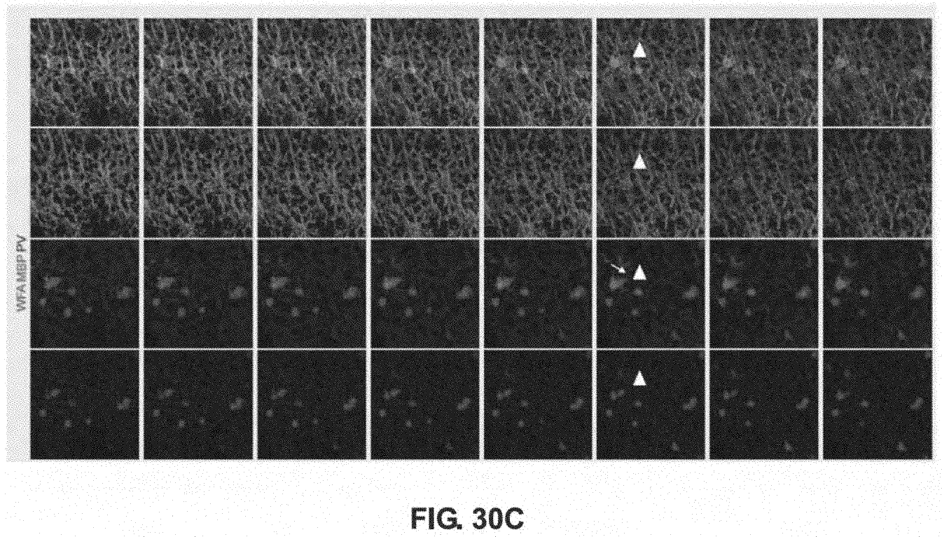

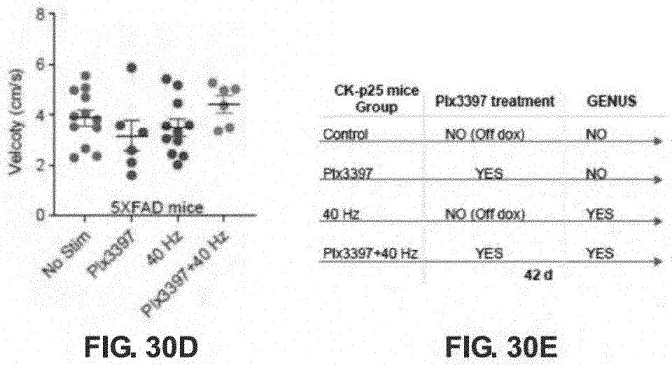

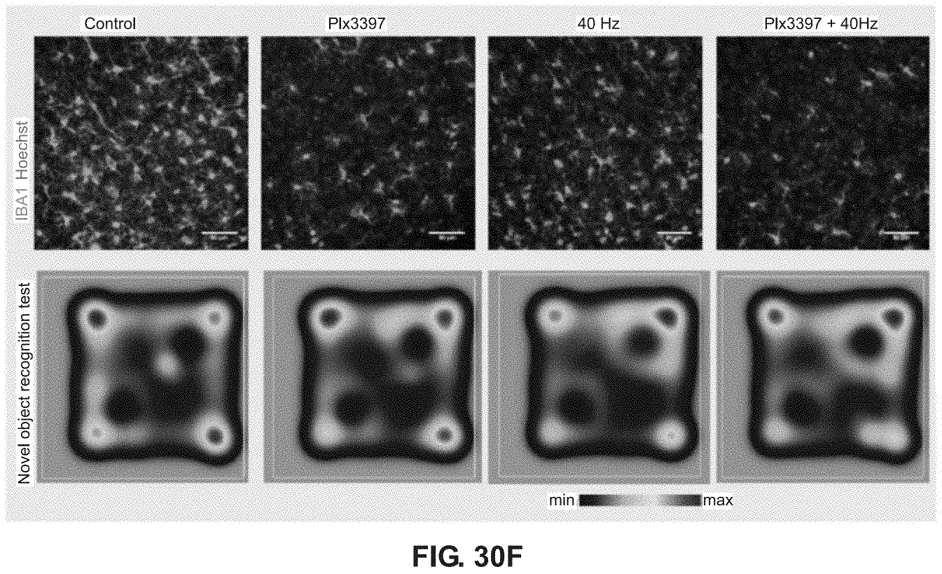

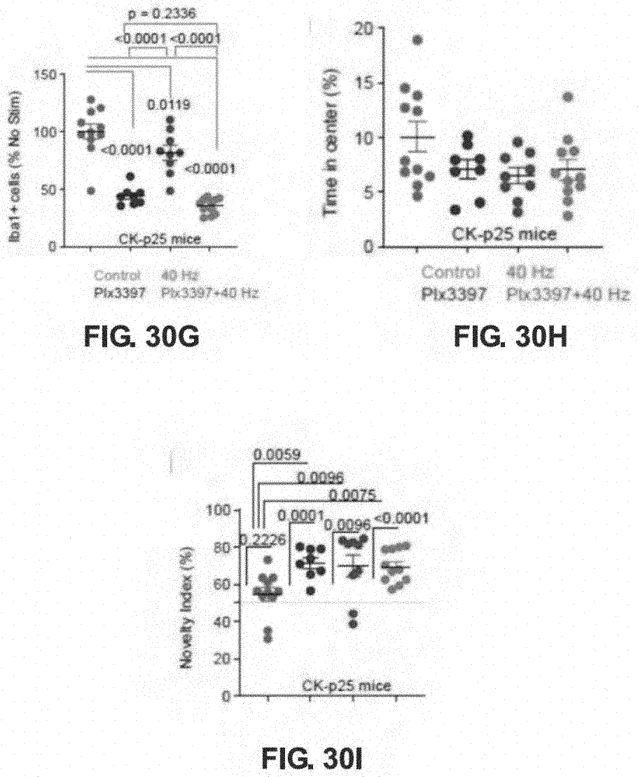

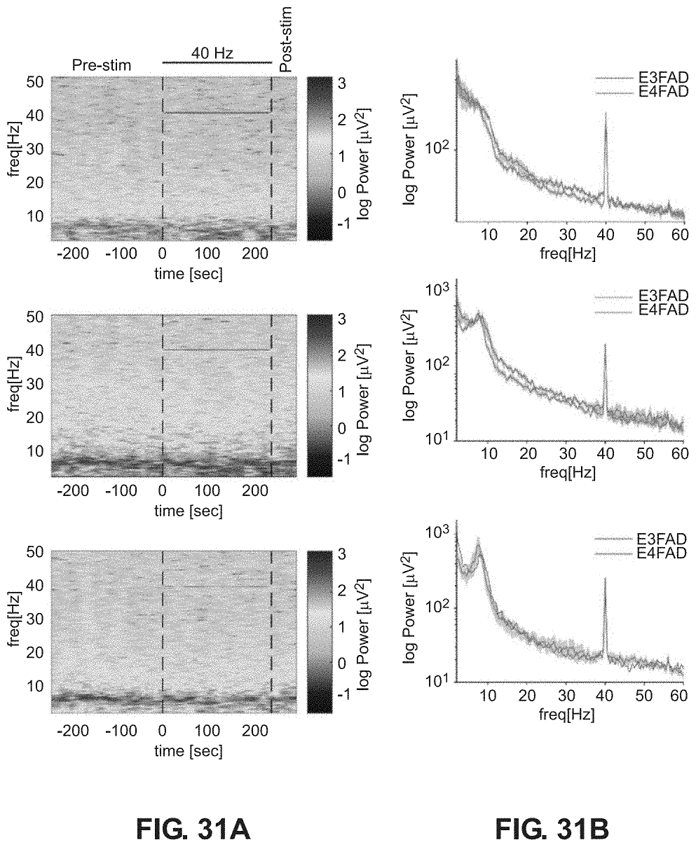

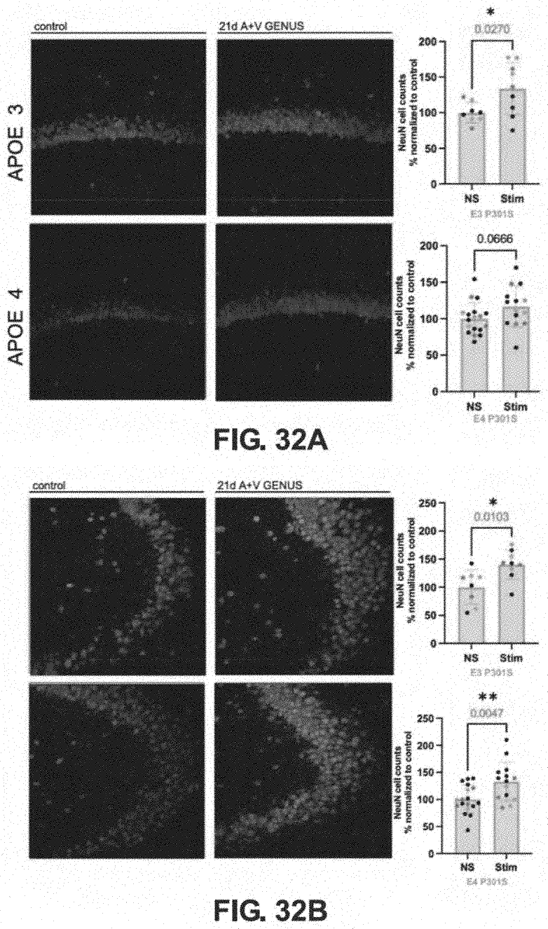

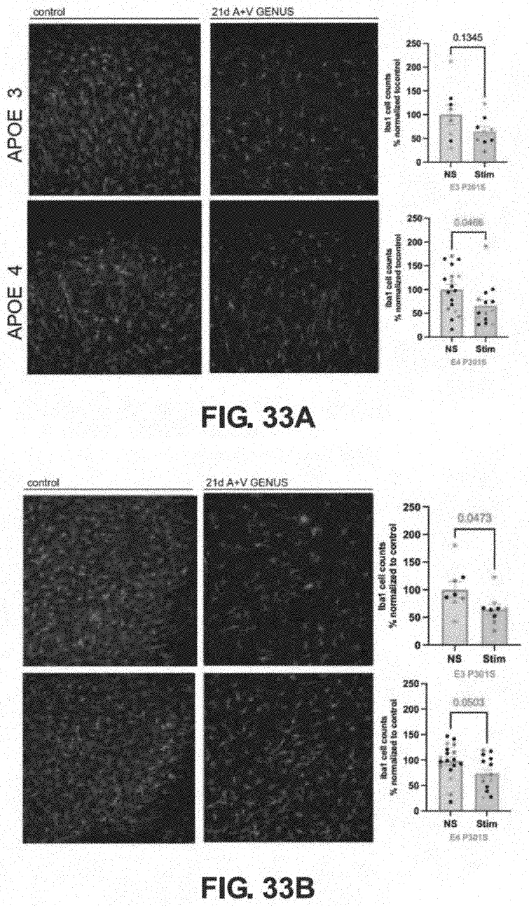

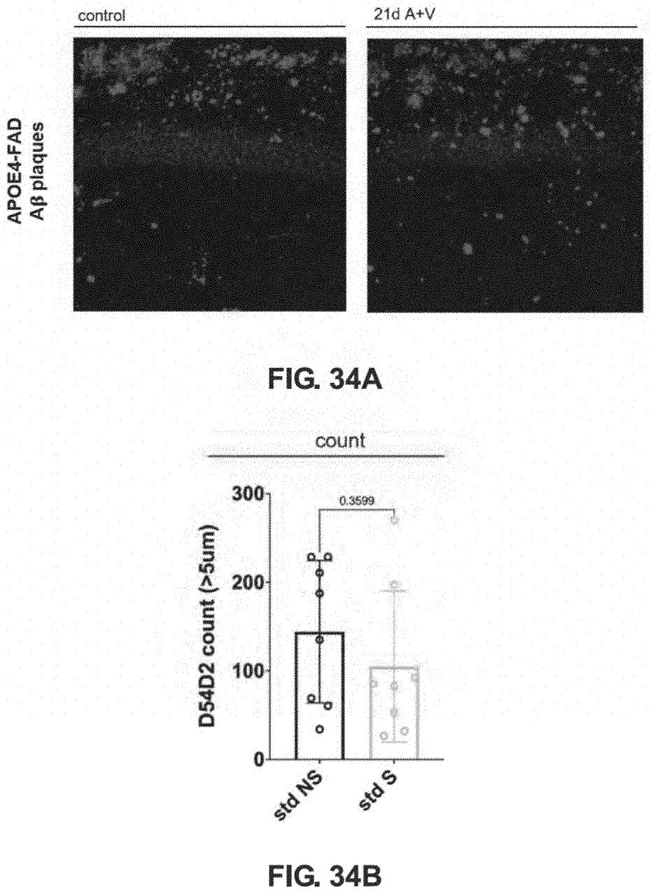

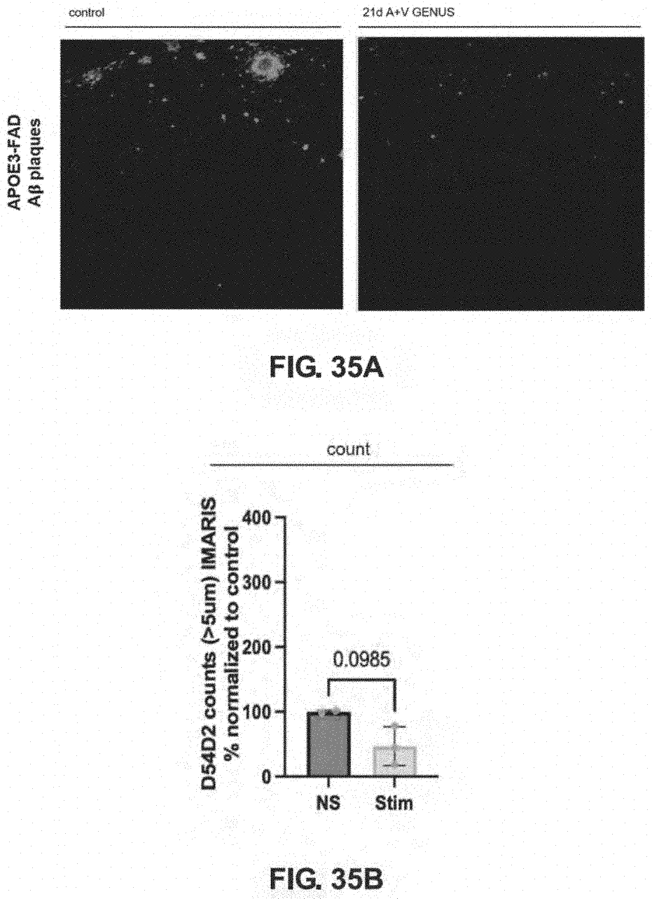

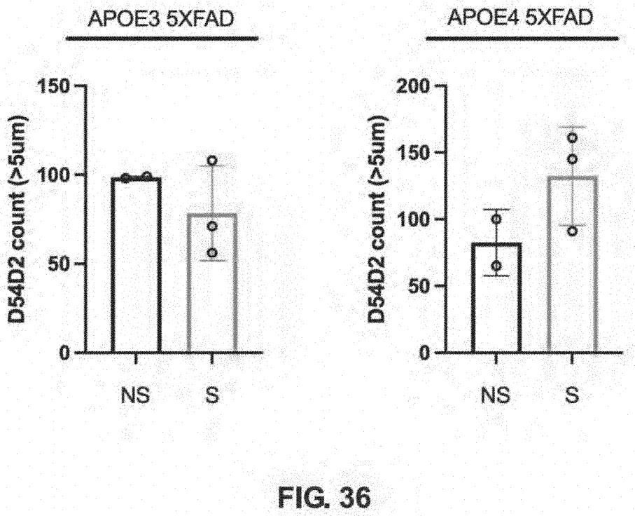

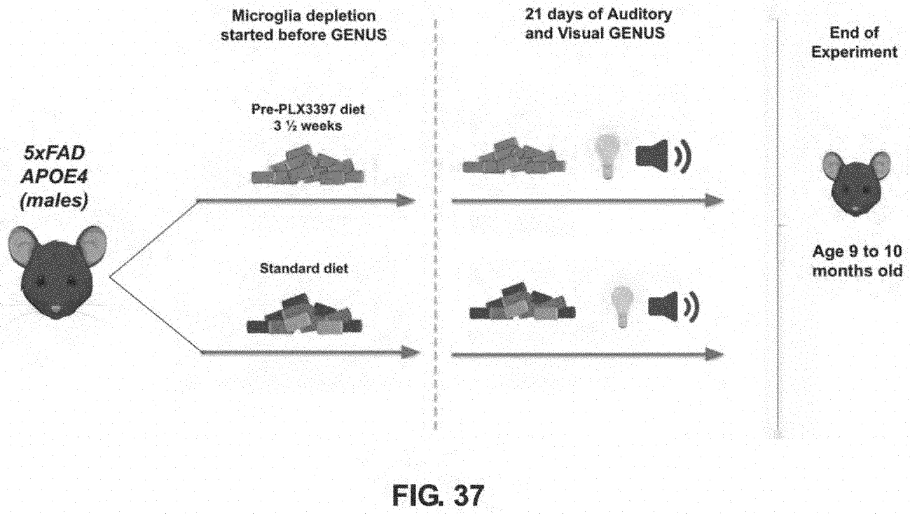

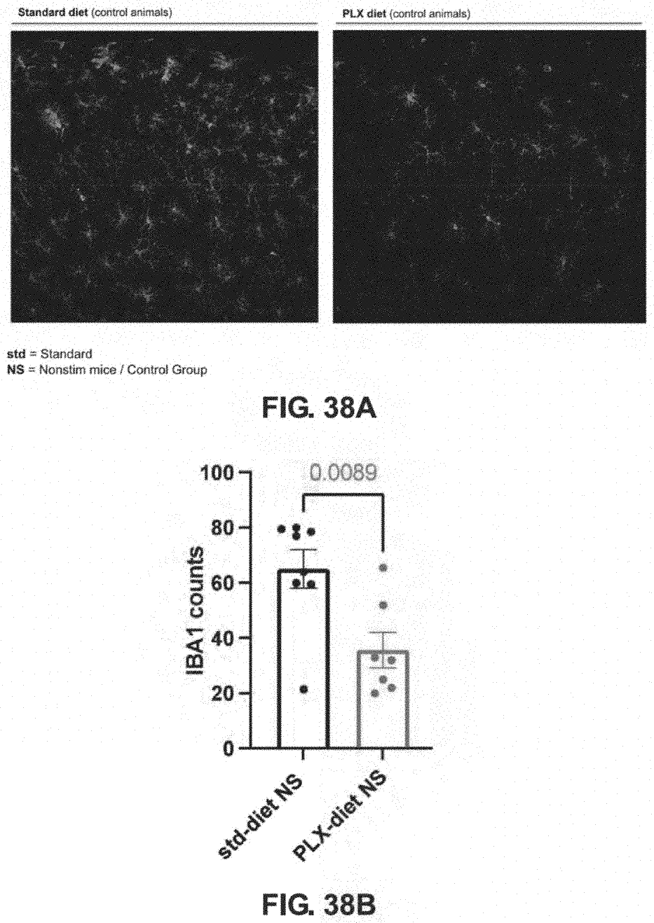

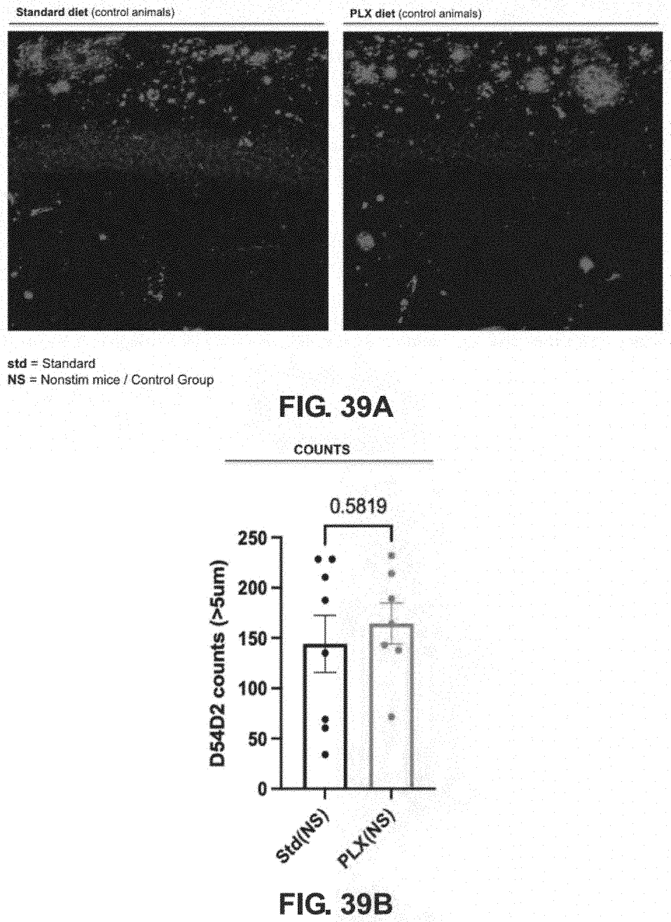

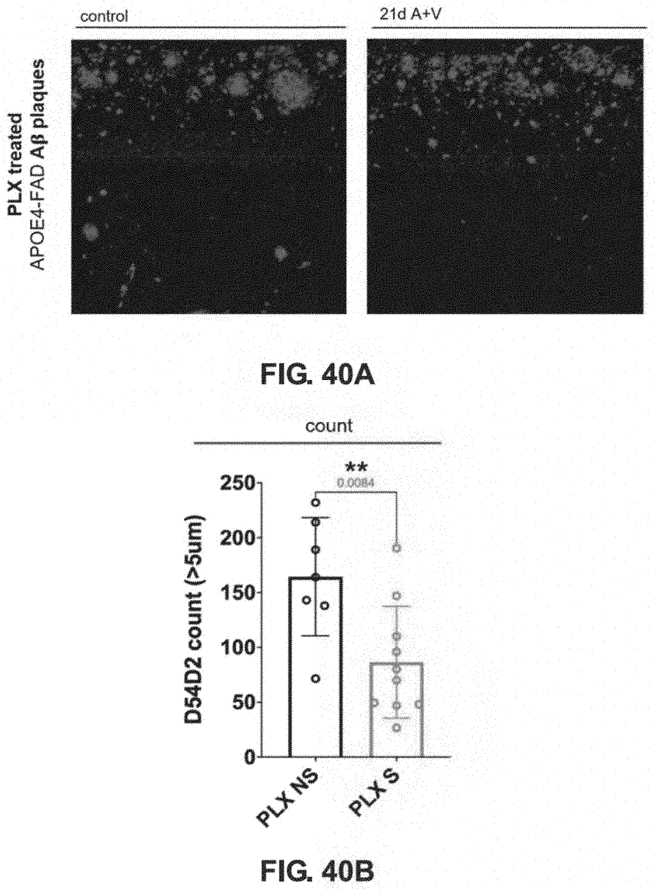

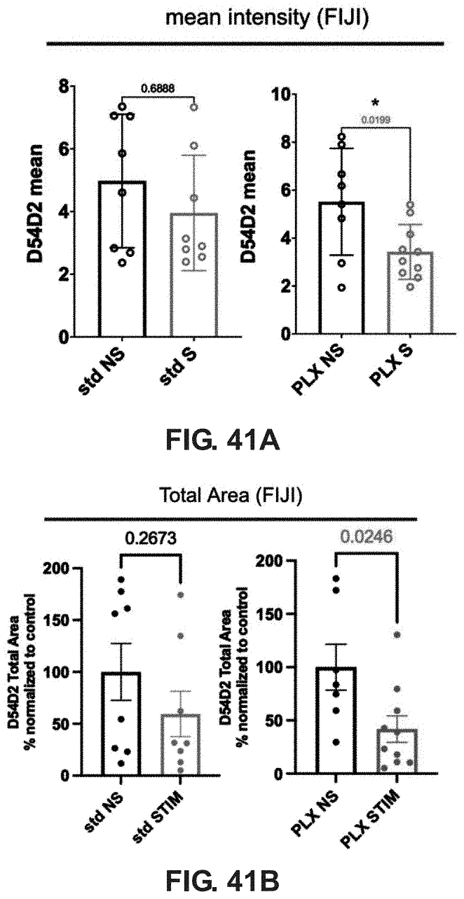

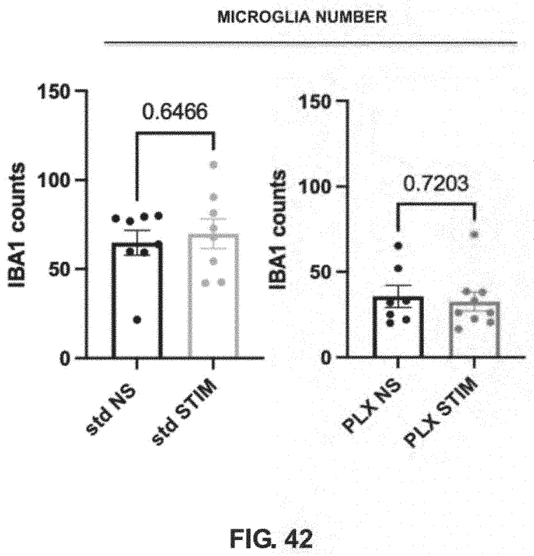

FIG. 25G shows summary graphs showing vGLUT1 puncta within WFA as % of no treatment control (nested ANOVA, F=3.300, p=0.0202).

FIG. 25H shows a summary graph showing the expression of MBP (ANOVA, F (3, 22)=0.5800, p=0.6343).

FIG. 25I shows a summary graph showing the expression of myelinated PV axons (ANOVA F (3, 83)=5.895, p=0.0011).

FIG. 25J shows a summary chart showing neuronal (NeuN) densities (ANOVA F (3,29)=3.072, p=0.0433).

FIG. 25K shows a schematic of and NOR test in 5×FAD, and mice occupancy heatmaps.

FIG. 25L shows the time spent in the center during OF (ANOVA, F (3, 31)=0.3847, p=0.7647) did not differ between groups

FIG. 25M shows the novelty index during NOR test was higher Plx3397+GENUS treated 5×FAD mice (ANOVA, F (3, 31)=3.456, p=0.0282).

FIGS. 26A-26D show CSF1R sensitive microglia elimination disrupts neural synchrony in 5×FAD mice.

FIG. 26A shows confocal images showing MAC2 signals in control and Plx3397 treated 5×FAD mice. Scale bar=50 μm.

FIG. 26B shows that MAC2+ signal did not differ between control and Plx3397 5×FAD mice.

FIG. 26C shows unprocessed raw LFP traces during theta-burst and gamma states in Plx3397 5×FAD mice. L2/3, L4, L5, & L6 indicate cortical layers 2/3, 4, 5, & 6, respectively.

FIG. 26D shows line plots show the mean (±s.e.m) spike rate of E-neurons (top) and I-neurons (bottom) pre-, during, and post-theta-burst from L2/3, L4, L5 and L6. Time zero represents theta-burst onset.

FIGS. 27A-27G show that chronic Plx3397 treatment impacts synaptic pathology in the hippocampus in 5×FAD mice. Scale bar=10, 50 or 100 m as indicated. N=5-6 mice per group, au=arbitrary units.

FIG. 27A show example confocal images showing IBA1, D54D2 amyloid, myelin basic protein (MBP), complementary molecule C1q, synaptophysin, and Wisteria floribunda agglutinin (WFA) in the control and Plx3397 administered 5×FAD mice.

FIG. 27B shows that Plx3397 reduced IBA1+ cells (unpaired t-test, t=5.290).

FIG. 27C shows that Plx3397 did not affect amyloid (t=0.4048).

FIG. 27D shows that Plx3397 did not affect MBP levels (t=0.8238).

FIG. 27E shows that Plx3397 reduced C1q levels (t=2.646).

FIG. 27F shows that Plx3397 increased synaptophysin (t=3.508).

FIG. 27G shows that Plx3397 increased WFA signals (t=4.455).

FIGS. 28A-28G show sensory evoked gamma oscillations in Plx3397 treated 5×FAD mice.

FIG. 28A shows representative LFP trace during 4 Hz entrainment in control 5×FAD mice.

FIG. 28B shows LFP waveforms as a function of 4 Hz stimulus in control 5×FAD mice.

FIG. 28C shows LFP trace (top) during pre-stimulation in Plx3397 treated 5×FAD mice. The corresponding wavelet LFP spectrogram before, during, and after acute 40 Hz stimulation. Note the reduction in theta-burst during the 40 Hz entrainment.

FIG. 28D shows LFP power spectrum in 5×FAD mice with or without Plx3397 administration for 50 days. Plx3397 administered 5×FAD mice exhibited clear 40 Hz entrainment during acute 60 sec stimulation.

FIG. 28E shows a summary graph showing the absolute power of 40 Hz entrainment in control and Plx3397 administered 5×FAD mice.

FIG. 28F shows a plot showing the percentage of total neurons phase-locked to theta oscillations based on circular Rayleigh statistics.

FIG. 28G shows plots showing the strength of phase locking between neuronal spiking and LFP theta. No significant effect was observed in E-neurons (F (1, 97)=0.6899, p=0.4082) and I-neurons (ANOVA, F (1, 43)=0.4873, p=0.4889) between baseline and 40 Hz entrainment.

FIGS. 29A-29H show that the administration of GENUS in Plx3397 treated 5×FAD improves extracellular matrix and myelination related genes in oligodendrocytes and/or microglia.

FIG. 29A shows a UMAP visualization of single cell (sc)RNA-seq from 11-month-old 5×FAD mice showing microglia clusters. Plots demonstrate scaled gene expression for cluster markers for microglia (Cx3cr1, Selplg, P2ry12, Tmem119).

FIG. 29B shows a UMAP visualization of single cell (sc)RNA-seq from 11-month-old 5×FAD mice showing oligodendrocytes clusters. Plots demonstrate scaled gene expression for cluster markers for oligodendrocytes (Olig1, Cldn11, Mal, MBP).

FIG. 29C shows the top 5 upregulated biological pathways in microglia.

FIG. 29D shows the top 5 upregulated biological pathways in oligodendrocytes.

FIG. 29E shows a volcano plot of differentially expressed genes in microglia. Red dots represent upregulated transcripts, while blue dots represent downregulated transcripts in Plx3397+GENUS compared to control 5×FAD mice. y-axes represent adjusted log 2 p-value for cluster changes. Genes involved in lipid metabolism & transport were upregulated in microglia after Plx3397+GENUS administration compared to Plx3397 administration alone.

FIG. 29F shows a volcano plot of differentially expressed genes in microglia. Red dots represent upregulated transcripts, while blue dots represent downregulated transcripts in Plx3397+GENUS compared to control 5×FAD mice. y-axes represent adjusted log 2 p-value for cluster changes. Genes involved in extracellular matrix organization were upregulated in microglia after Plx3397+GENUS administration compared to Plx3397 administration alone.

FIG. 29G shows a volcano plot of differentially expressed genes in oligodendrocytes. Red dots represent upregulated transcripts, while blue dots represent downregulated transcripts in Plx3397+GENUS compared to control 5×FAD mice. y-axes represent adjusted log 2 p-value for cluster changes. In oligodendrocytes, genes involved in extracellular matrix architecture were upregulated after Plx3397+GENUS administration compared to Plx3397 administration alone.

FIG. 29H shows a volcano plot of differentially expressed genes in oligodendrocytes. Red dots represent upregulated transcripts, while blue dots represent downregulated transcripts in Plx3397+GENUS compared to control 5×FAD mice. y-axes represent adjusted log 2 p-value for cluster changes. In oligodendrocytes, genes involved in myelination were upregulated after Plx3397+GENUS administration compared to Plx3397 administration alone.

FIGS. 30A-30I show that chronic administration of Plx3397 and GENUS improved novel object recognition memory in CK-p25 mouse model of neurodegeneration.

FIG. 30A shows representative confocal images showing WFA and IBA1 (scale bar=20 μm).

FIG. 30B shows a plot showing the WFA signal within IBA1 (ANOVA F=17.96, p<0.0001).

FIG. 30C shows a representative serial single plane confocal images show WFA, MBP, and PV. Note that MBP signals around the axonal process of PV interneurons are evident immediately after WFA but not within WFA. This suggests a multifaceted regulation of the PV axon through myelination and PNN.

FIG. 30D shows a plot showing the velocity of mice during a novel object recognition memory test in 5×FAD mice. ANOVA F (3, 31)=1.437, p=0.2508.

FIG. 30E shows an experiment outline to induce p25 expression in CK-p25 mice and subject the animals to Plx3397, GENUS, or Plx3397+GENUS treatments.

FIG. 30F shows confocal images of IBA1 from the visual cortex in CK-p25 mice (scale bar=50 μm) (top) and CK-p25 mice occupancy heatmap during the open field test (bottom).

FIG. 30G shows IBA1+ microglia were significantly reduced in Plx3397, GENUS Plx3397+GENUS CK-p25 mice (n=8-11 mice/group; ANOVA, F (3, 35)=40.93, p<0.0001).

FIG. 30H shows a plot showing time spent (% of total time) in the center of an open field arena. Plx3397, GENUS or Plx3397+GENUS did not have any effect on the open field exploration (ANOVA, F (3, 35)=2.563, p=0.0704).

FIG. 30I shows novelty index in NOR test (ANOVA, F (3, 35)=4.224, p=0.0119).

FIGS. 31A-31B show 40 Hz Combined Visual and Auditory Stimulation Entrains Gamma Oscillations in ApoE 5×FAD mice. Mean values, standard error of the mean, (ApoE3 5×FAD: n=4; ApoE4 5×FAD: n=5).

FIG. 31A shows a representative spectrogram of EEG signals recorded simultaneously from frontal (top), somatosensory (middle) and visual (bottom) derivations in an ApoE4x5×FAD mouse.

FIG. 31B shows EEG power density during 40 Hz stimulation in frontal (top), somatosensory (middle) and visual (bottom) derivations.

FIG. 32A shows neuronal nuclei staining (NeuN) in the CA1 region of the hippocampus in APOE3 and APOE4 tau mouse models of AD. Following 21 days of auditory and visual combined (A+V) GENUS, a significant increase in NeuN numbers is observed compared to control animals that did not receive GENUS. Quantification (right), students ttest, * p=0.027 (APOE3 tau), p=0.0666 (APOE4 tau).

FIG. 32B shows neuronal nuclei staining (NeuN) in the CA3 region of the hippocampus in APOE3 and APOE4 tau mouse models of AD. Following 21 days of auditory and visual combined (A+V) GENUS, a significant increase in NeuN numbers is observed compared to control animals that did not receive GENUS. Quantification (right), students ttest, * p=0.0103 (APOE3 tau), ** p=0.0047 (APOE4 tau).

FIGS. 33A-33B shows that APOE-KI animals show reduced microglia following 21d A+V GENUS.

FIG. 33A shows a decrease in Iba1+ cell numbers in CA1 observed compared to control animals that did not receive GENUS following 21 days of auditory and visual combined (A+V) GENUS. Quantification (right), students ttest, p=0.1345 (APOE3 tau), * p=0.0466 (APOE4 tau).

FIG. 33B shows a significant decrease in Iba1+ cell numbers in CA3 observed compared to control animals that did not receive GENUS following 21 days of auditory and visual combined (A+V) GENUS. Quantification (right), students ttest, * p=0.0473 (APOE3 tau), p=0.0503 (APOE4 tau).

FIGS. 34A-34B show that APOE4-KI 5×FAD animals do not show reduction in amyloid burden following 21 days A+V GENUS.

FIG. 34A shows images of hippocampal slices of animals treated with 21 days A+V GENUS (right panel) or control animals (left panel) that did not receive GENUS were stained for the amyloid antibody D54D2 to identify amyloid plaques (red).

FIG. 34B shows a quantification showing that the plaque number was not significantly reduced following 21d A+V GENUS. Students ttest, ns p=0.3599.

FIGS. 35A-35B show that APOE3-KI 5×FAD animals appear to show reduction in amyloid burden following 21 days A+V GENUS.

FIG. 35A shows images of hippocampal slices of animals treated with 21 days A+V GENUS or control animals that did not receive GENUS were stained for the amyloid antibody D54D2 to identify amyloid plaques (green).

FIG. 35B shows a quantification showing that the plaque number trended to reduction. Students ttest, ns p=0.0985.

FIG. 36 shows an independent cohort of younger (6 mo) APOE-KI 5×FAD animals treated with 21d A+V GENUS suggests APOE3 animals (left) that receive GENUS (S) may reduce amyloid burden compared to control animals (NS), while APOE4 (right) animals do not.

FIG. 37 shows a schematic of experimental set up to examine effect of microglia depletion using CSF1r inhibitor PLX3397 on APOE4-KI 5×FAD outcomes following 21 days A+V GENUS.

FIG. 38A shows hippocampus sections from APOE4-KI 5×FAD animals on PLX3397-containing diet showed significantly reduced microglia numbers by Iba1+ staining, compared to standard diet (std) controls.

FIG. 38B shows the quantification of the results of FIG. 38A. Students ttest, ** p=0.0089.

FIG. 39A shows that amyloid load (D54D2 staining, red) in aged (9-10 month old) APOE4-KI 5×FAD animals is not significantly altered by microglia depletion alone.

FIG. 39B shows the quantification of the results of FIG. 39A. Students ttest, ns p=0.5819.

FIGS. 40A-40B show that the combinatorial application of microglia depletion with PLX3397 diet and 21 days A+V GENUS results in significant reduction in amyloid plaque number in APOE4-KI 5×FAD animals.

FIG. 40A shows D54D2 amyloid plaque staining in the hippocampus CA1.

FIG. 40B shows the quantification of the results of FIG. 40A. Students ttest, ** p=0.0084.

FIG. 41A shows that the combinatorial application of microglia depletion with PLX3397 diet and 21 days A+V GENUS results in significant reduction in amyloid staining mean intensity in APOE4-KI 5×FAD animals, compared to standard diet (no depletion). Students ttest, * p<0.05.

FIG. 41B shows that the combinatorial application of microglia depletion with PLX3397 diet and 21 days A+V GENUS results in significant reduction in total area in APOE4-KI 5×FAD animals. Students ttest, * p=0.0246.

FIG. 42 shows that microglia numbers (Iba1+ cell counts) are not further modified by GENUS following PLX3397-mediated microglia depletion. Students ttest, ns p=0.7203.

### DETAILED DESCRIPTION

All combinations of the foregoing concepts and additional concepts are discussed in greater detail below (provided such concepts are not mutually inconsistent) and are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

The present disclosure is directed generally to non-invasively administering a stimulus (e.g., visual, auditory and/or tactile) to a subject, wherein the stimulus is in a range of frequencies that induces gamma oscillations in the brain of the subject, in combination with administering one or more pharmacological agents (e.g., drugs) to the subject, to significantly ameliorate one or more pathological conditions in the brain of the subject.

In one example implementation, the present disclosure provides methods, devices, and systems for treating Alzheimer's disease in a subject in need thereof that includes administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, administering derivatives of CSF1R inhibitors or allosteric modulators of CSF1R in combination with 20 Hz to 60 Hz stimulus may provide effects in improving daily life activities in subjects with neurological or brain/peripheral tumor.

In another aspect, the present disclosure provides methods, devices, and systems for reducing the number of microglia in at least one brain region of a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. When the CSF1R administration is done systemically, the reduction in number of microglia, and/or the other effects disclosed herein, can be observed substantially throughout the brain of the subject. Without being limited by theory, combined administration of an inhibitor and stimulus as disclosed herein may provide an effect throughout the body of the subject due to the fact that the inhibitor is administered orally (i.e., systemically).

In another aspect, the present disclosure provides methods, devices, and systems for increasing synaptic density in at least one brain region of a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may improve synaptic homeostasis and prevent further synaptic loss throughout the brain of the subject with advanced disease state.

In another aspect, the present disclosure provides methods, devices, and systems for increasing neuronal density in at least one brain region of a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may preserve neuronal density or prevent the loss of neurons throughout the brain of the subject with advanced disease state.

In another aspect, the present disclosure provides methods, devices, and systems for reducing neuroinflammation in at least one brain region of a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may reduce or mitigate inflammation throughout the brain and the body of the subject due to the fact that the treatment is administered systemically. Outside the central nervous system, combined administration of an inhibitor and stimulus as disclosed herein may mitigate inflammation in joints, guts, intestines, respiratory system and muscles of the subject.

In another aspect, the present disclosure provides methods, devices, and systems for reducing expression of genes associated with protein synthesis in microglia in a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may regulate protein synthesis rate and genes involved in protein synthesis in multiple cell types, including microglia, astrocytes, oligodendrocytes and neurons. Further, it may impact the protein synthesis mechanisms in non-neural cell-types throughout the body of the subject including, but not limited to, muscle cells, skin cells, intestinal cells and other cells alike due to the inhibitor being administered orally (i.e., systemically).

In another aspect, the present disclosure provides methods, devices, and systems for increasing expression of genes associated with transport of low-density lipoprotein by microglia in a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may regulate general lipoprotein transport and the downstream function of lipoproteins throughout the brain and the body of the subject.

In another aspect, the present disclosure provides methods, devices, and systems for increasing expression of genes associated with vesicle organization (e.g., one or more of vesicle packaging, vesicle transport, release of vesicles such as synaptic vesicles and endosomal vesicles, and/or the like) in a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may regulate both intracellular vesicles and extracellular vesicles such as exosomes, and this latter can impact non-physical cell-cell communications in the subject.

In another aspect, the present disclosure provides methods, devices, and systems for increasing phase locking of neuronal spikes to gamma oscillations in at least one brain region of a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may regulate spike rate and spike rhythmicity of excitatory and inhibitory neurons in the cortex, hippocampus and other brain regions. Further, combined administration of an inhibitor and stimulus as disclosed herein may improve aberrant oscillatory activity measured in local field potentials or electroencephalograms (EEG) of the subject.

In another aspect, the present disclosure provides methods, devices, and systems for increasing the density of perineuronal net of neurons in at least one brain region of a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may improve the functions of neurons covered by the perineuronal nets and thus oscillations. The brain region(s) as disclosed in these aspects can include the visual cortex, the hippocampus, and/or other cortical regions.

In another aspect, the present disclosure provides methods, devices, and systems for increasing expression of genes associated with extracellular matrix organization around neurons in a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may improve the overall extracellular space and thus brain mass in the subject.

In another aspect, the present disclosure provides methods, devices, and systems for increasing expression of transcription factors such as Mef2c associated with improved neuronal and circuit health in a subject for treating Alzheimer's disease in the subject in need thereof. This includes administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, the treatment may improve gene expression program through altering transcription factors in many cell types, including excitatory neurons, interneurons, and parvalbumin interneurons.

In another aspect, the present disclosure provides methods, devices, and systems for increasing myelination in a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, the treatment may improve myelination of excitatory neurons and interneurons and enhance the myelination process of microglia and oligodendrocytes throughout the brain in the subject.

In another aspect, the present disclosure provides methods, devices, and systems for improving memory in a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, combined administration of an inhibitor and stimulus as disclosed herein may improve the quality of life including sleep.

In another aspect, the present disclosure provides methods for providing a device that administers a stimulus to a subject during use of the device. The can have a stimulus has a frequency of from about 20 Hz to about 60 Hz. The subject can previously and/or concurrently have been administered an inhibitor including a colony-stimulating factor-1 receptor (CSF1R) inhibitor or a colony-stimulating factor-1 (CSF1) inhibitor.

In another aspect, the present disclosure provides methods, devices, and systems for phase locking of neurons to theta oscillations in a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, the combined administration of CSF1R inhibitor and stimulus as disclosed herein can improve neural phase locking during sleep oscillations and sleep quality in subjects.

In another aspect, the present disclosure provides methods, devices, and systems for increasing myelination in at least one brain region of a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, the combined administration of CSF1R inhibitor and stimulus as disclosed herein can be used to improve outcomes in subjects with brain tumor or trauma because brain tumors robustly associated with proliferations and higher densities of glial cells and the combined administration reduces glial populations in subjects.

In another aspect, the present disclosure provides methods, devices, and systems for reducing microglia in at least one brain region of a subject for treating Alzheimer's disease in the subject in need thereof. This is accomplished by administering an inhibitor including a colony-stimulating factor-1 receptor (CSF1R) inhibitor or a colony-stimulating factor-1 (CSF1) inhibitor to the subject, and also non-invasively administering a stimulus to the subject having a frequency from about 20 Hz to about 60 Hz. Without being limited by theory, the combined administration of CSF1R inhibitor and stimulus as described herein can be used to improve outcomes in epilepsy and seizures because the approach improves overall phase-locking and reduces aberrant neural synchrony that occurs in subjects that suffer from epileptic seizures.

In some cases, the subject has at least one Apolipoprotein E4 (APOE4) allele. Said another way, the subject can have one copy or two copies of the APOE4 gene. Inheritance of one or two copies of APOE4 can increase risk for Alzheimer's Disease in a dose dependent manner, and similarly decrease the age of onset for AD. Multiple brain cell types are affected by APOE4, including microglia, the immune cells of the brain. Bearing one or two copies of APOE4 may therefore impact the functioning of these brain cell types, and interfere with treatment outcomes. Without being limited by theory, combined administration of cell type specific modulation, including a colony-stimulating factor-1 receptor (CSF1R) inhibitor, and stimulus as disclosed herein may improve treatment outcomes.

Each of the aspects provided herein can further encompass manufacture and/or use of a device and/or system for the stated objective(s), i.e., for one or more of treating Alzheimer's disease, reducing number of microglia in at least one brain region, increasing synaptic density in at least one brain region, increasing neuronal density in at least one brain region, reducing neuroinflammation in at least one brain region, reducing expression of genes associated with protein synthesis in microglia, increasing expression of genes associated with transport of low-density lipoprotein by microglia, increasing expression of genes associated with vesicle organization, increasing phase locking of neuronal spikes to gamma oscillations in at least one brain region, increasing the density of perineuronal net of neurons in at least one brain region, increasing expression of genes associated with extracellular matrix organization around neurons, increasing expression of transcription factors associated with improved neuronal and circuit health, increasing myelination, improving memory, locking of neurons to theta oscillations, increasing myelination in at least one brain region, and reducing microglia in at least one brain region in a subject having at least one APOE4 allele.

The inhibitor can be administered orally such as, for example, with or without food. For example, 0.6% PLX-3397 can be included in diet/chow. In some cases, the PLX-3397 can be administered intraperitoneally.

In some cases, the inhibitor is a CSF1R inhibitor, and includes pexidartinib, also sometimes referred to as PLX-3397. In some cases, the inhibitor is a CSF1R inhibitor and includes one or more of pexidartinib, bosutinib, imatinib, gefitinib, ruxolitinib, dasatinib, sunitinib, erlotinib, lapatinib, pazopanib, crizotinib, vemurafenib, PLX7486, ARRY-382, Edicotinib, BLZ945, Emactuzumab, AMG 820, Cabiralizumab, and IMC-CS4.

In some cases, the inhibitor and the stimulus can be administered starting the same day. In some cases, the inhibitor can be administered prior to the administration of the stimulus such as, for example, one day before, two days before, a week before, 10 days before, 20 days before, 25 days, 40 days, 50 days, or more before, including all values and sub-ranges in between. In some cases, the inhibitor can then continue to be administered concurrently with the administration of the stimulus. In other cases, the administration of the inhibitor is stopped prior administration of the stimulus. Said another way, the timing of administration of the inhibitor and administration of the stimulus can partially overlap, completely overlap, or be mutually exclusive.

The stimulus can be administered invasively and/or non-invasively. The term “non-invasive,” as used herein, refers to methods, devices, and systems which do not require surgical intervention or manipulations of the body, such as injection or implantation of a composition or a device. The term “invasive,” as used herein, refers to methods, devices, and systems which do require surgical intervention or manipulations of the body. Non-limiting examples of non-invasive administration of stimulus can include audio, visual (e.g., flickering lights), haptic stimulation, and/or the like. Non-limiting examples of invasive administration of stimulus can include visual, audio, and/or haptic stimulations combined with an injection or implantation of a composition (e.g., a light-sensitive protein) or a device (e.g., an integrated fiber optic and solid-state light source). Other examples of invasive administration can include magnetic and/or electrical stimulation via an implantable device or a device disposed on the body of the subject.

The stimulus may include any purposive, detectable change in the internal (e.g., when the stimulus is administered invasively) or external (e.g., when the stimulus is administered non-invasively) environment of the subject that directly or ultimately has the desired effect. For example, the stimulus may be designed to at least stimulate electromagnetic radiation receptors (e.g., photoreceptors, infrared receptors, and/or ultraviolet receptors) and sound receptors, and may further stimulate one or more of mechanoreceptors (e.g., mechanical stress and/or strain), nociceptors (i.e., pain), electroreceptors (e.g., electric fields), magnetoreceptors (e.g., magnetic fields), hydroreceptors, chemoreceptors, thermoreceptors, osmoreceptors, or proprioceptors (i.e., sense of position). The absolute threshold or the minimum amount of sensation needed to elicit a response from such receptors may vary based on the type of stimulus and the subject. In some embodiments, the stimulus is adapted based on individual sensitivity to the stimulus.

For example, the stimulation may be visual (e.g., a flickering light), as generally disclosed in PCT Publication Nos. 2017/091698, 2019/074637, and/or 2019/075094 the entire disclosure of each of which is incorporated herein by reference. In some cases, the stimulation may include an auditory stimulus and/or a haptic/tactile stimulus, as generally disclosed in the aforementioned applications. Each of the haptic/tactile stimulus, auditory stimulus, and the visual stimulus can independently be non-invasive, or invasive, or a combination thereof.

The stimulus can have a frequency of less than about 20 Hz, about 20 Hz, about 30 Hz, about 40 Hz, about 50 Hz, about 60 Hz, or more than 60 Hz, including all values and sub-ranges in between. In particular embodiments, the stimulus is a visual stimulus including a light flashing at about 20 Hz to about 60 Hz. In some embodiments, the light is flashing at about 40 Hz. In some embodiments, the subject receives (e.g., is placed in a chamber with or wears a light blocking device emitting) about 20 Hz to about 100 Hz flashing light, or about 20 Hz to about 50 Hz flashing light or about 35 Hz to about 45 Hz flashing light, or about 40 Hz flashing light.

The stimulus can be applied for a duration of about 15 minutes, about 30 minutes, about an hour, about two hours, about four hours more than four hours, including all values and sub-ranges in between. In another aspect, the stimulus can be applied for a predetermined duration (e.g., about an hour) once or daily for a week, for two weeks, three weeks, a month, or more than a month, including all values and sub-ranges in between. In some cases, the stimulus can be applied for about an hour a day for at least three weeks.

Systems and devices for delivering the stimulus as disclosed herein can generally include any suitable stimulus emitting and/or delivery device. Examples of such devices for generating and/or delivering a visual stimulus can include, but are not limited to, flash lamps, pulsed lasers, light emitting diodes including laser diodes (and generally, any solid-state light source), intense pulsed light (IPL) sources, a device screen (e.g., the screen of a Smartphone, a laptop, a desktop computer, and/or the like), combinations thereof, and/or the like. Examples of such devices for generating and/or delivering an audio stimulus can include, but are not limited to, electroacoustic transducers, speakers, headphones, and/or the like. Examples of such devices for generating and/or delivering a haptic stimulus can include, but are not limited to, actuators (including eccentric rotating mass actuators, linear resonant actuators, magnetic voice coils, piezoelectric actuators, and/or the like), motors, focused ultrasound, and/or the like.

By way of example, in some embodiments, the visual stimulus can include repeated 12.5 ms light on then 12.5 ms light off. As another example, the light emitting device can include a light-emitting diode with 40-80 W power. As yet another example, the visual stimulus can include a light flickered at 40 Hz for 10 s period with a duty cycle of about 10% to about 80%.

In some cases, systems and devices for delivering the stimulus can also generally include a processor and a memory/database. All components of the systems and devices can be in communication with each other, including with the stimulus-emitting/delivery device. It will also be understood that the database and the memory can be separate data stores. In some embodiments, the memory/database can constitute one or more databases. Further, in other embodiments, at least one database can be external to the system/device. The system/device can also include one or more input/output (I/O) interfaces (not shown), implemented in software and/or hardware, for other components of the system/device, and/or external to the system/device, to interact with the system/device.

The memory/database can encompass, for example, a random access memory (RAM), a memory buffer, a hard drive, a database, an erasable programmable read-only memory (EPROM), an electrically erasable read-only memory (EEPROM), a read-only memory (ROM), Flash memory, and/or so forth. The memory/database can store instructions to cause the processor to execute processes and/or functions associated with the system/device. For example, the memory/database can store stimulus parameters (e.g., frequency, amplitude, duty cycle, etc.), processor executable instructions to control the stimulus-emitting device to emit the stimulus according to the stimulus parameters, and/or the like.

The processor can be any suitable processing device configured to run and/or execute a set of instructions or code associated with the system/device. The processor can be, for example, a general purpose processor, a Field Programmable Gate Array (FPGA), an Application Specific Integrated Circuit (ASIC), a Digital Signal Processor (DSP), and/or the like.

### Example 1

### INTRODUCTION

Alzheimer's disease (AD) is a debilitating and highly prevalent brain disorder that accounts for 60-80% of dementia cases, with more than 20% of people over age 75 being affected. There is a pressing need to both understand the mechanisms and find treatments for AD. Recent studies have used 40 Hz visual and/or auditory stimulation in a paradigm termed Gamma ENtrainment Using Sensory stimuli (GENUS). Using in vivo electrophysiology, it was confirmed that GENUS noninvasively induced neural oscillations at 40 Hz in multiple AD mouse models including 5×FAD, Tau P301S and CK-p25 mice. Significant reductions in Aβ peptides and amyloid plaque levels were found, as well as effects on microglia, astrocytes, and the brain vasculature after GENUS. It was also found that chronic GENUS in these mouse models reduced neuroinflammation, phosphorylation of tau protein, neurodegeneration, and loss of synapses while improving cognitive performance. In addition, other work demonstrated that chronic GENUS improves network connectivity and memory in human AD. These findings implicate multiple microglial changes in the beneficial effects of the GENUS response. Further, pharmacological reduction of microglia by colony-stimulating factor receptor-1 (CSF1R) inhibition also produced protective effects in mouse models of AD. Therefore, the primary goal of this research is to elucidate the importance and roles of microglia in the GENUS response. Specifically, whether the microglia reduction by CSF1R inhibitor (Plx3397) treatment together with GENUS will reduce AD-associated pathology while improving neuronal network and cognitive function was tested.

Results

Combined Administration of CSF1 Inhibitor and GENUS Improves Synaptic Density.

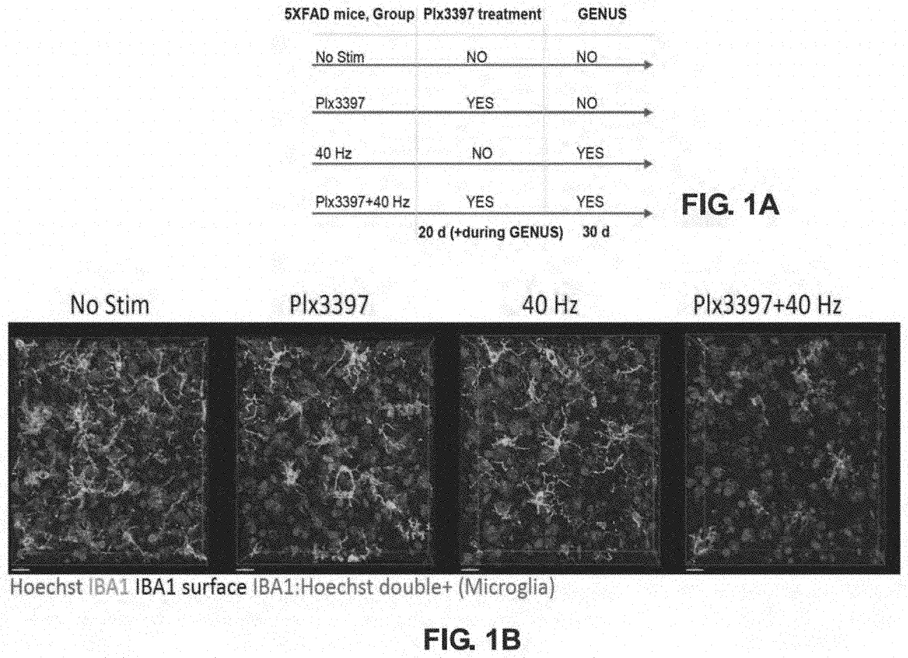

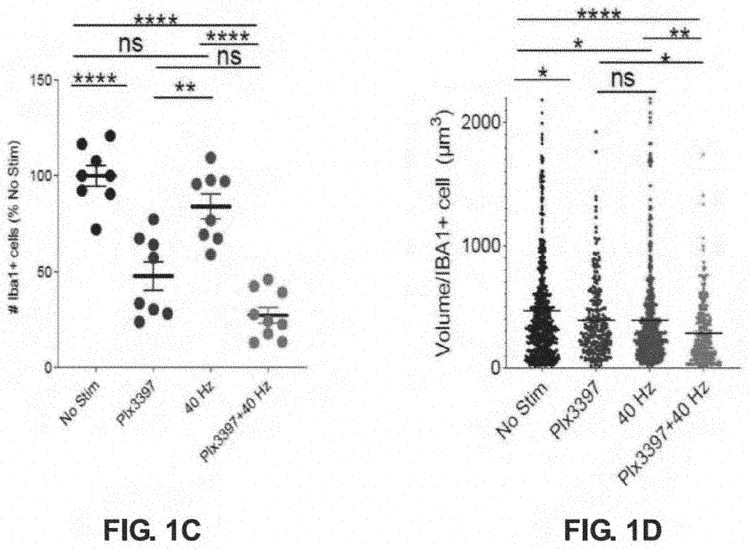

Experiments were performed to reduce microglia from 10-month-old 5×FAD mice, transgenic mice that overexpress human APP and PSEN1 genes harboring 5 AD-associated mutations, prior to GENUS treatment. Specifically, 5×FAD mice were treated, via oral delivery in mouse chow, with the selective CSF1R/c-kit/FLT3 inhibitor (Plx3397, Medkoo, irradiated and premixed into chow at 600 ppm by Envigo) that has been shown to eliminate microglia in vivo. Following 20 days of administration of Plx3397 chow, untreated- and Plx3397-treated 5×FAD mice (which continued Plx3397 administration) were subjected to 30 days of daily GENUS (FIG. 1A). Following completion of these treatments, microglial density in the visual cortex was compared between 1) untreated (No Stim), 2) Plx3397 treated, 3) GENUS treated and 4) Plx3397+GENUS treated 5×FAD mice (FIG. 1B). A significant reduction in IBA1+(Wako Chemicals, #019-19741) positive microglia was found with Plx3397, and Plx3397+GENUS treatment (ANOVA, F (3,29)=32.27, P<0.0001; N=8-9 mice per group) (FIGS. 1B, 1C). Further, a synergistic effect in Plx3397+GENUS treated mice was observed, which had significantly lower volume of microglia than mice receiving either treatment alone (Nested ANOVA, F (3,29)=13.12, P<0.0001) (FIG. 1D).

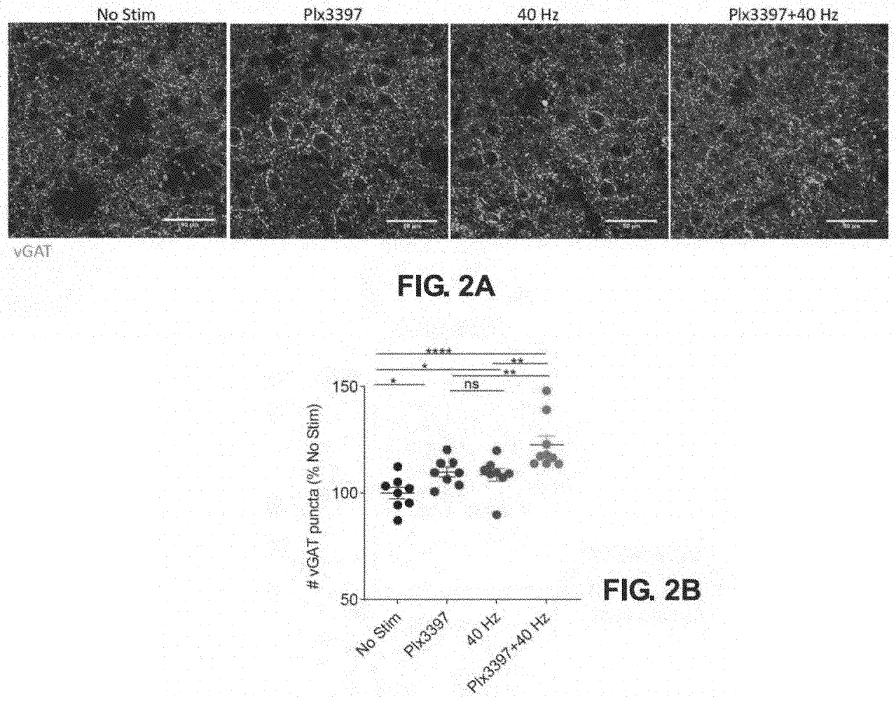

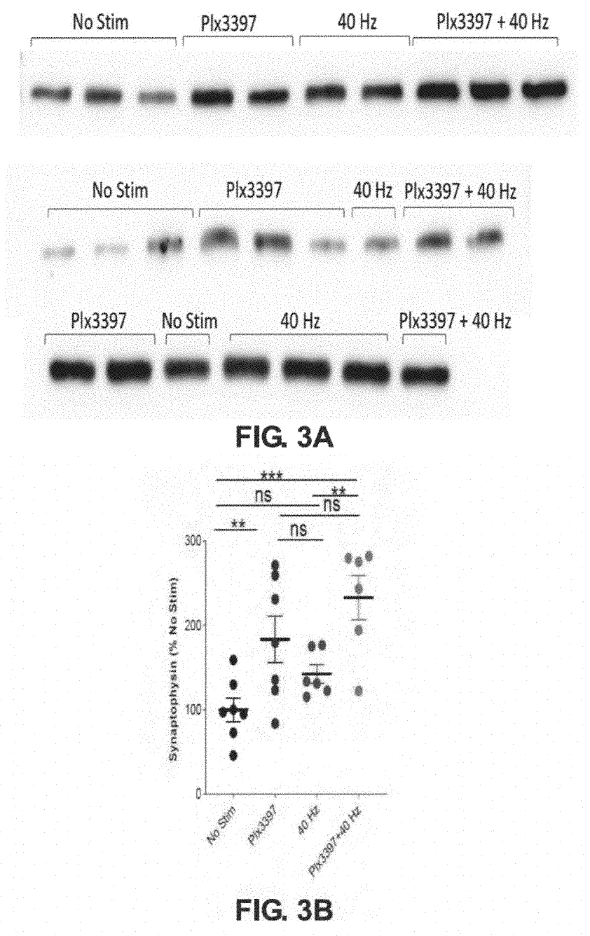

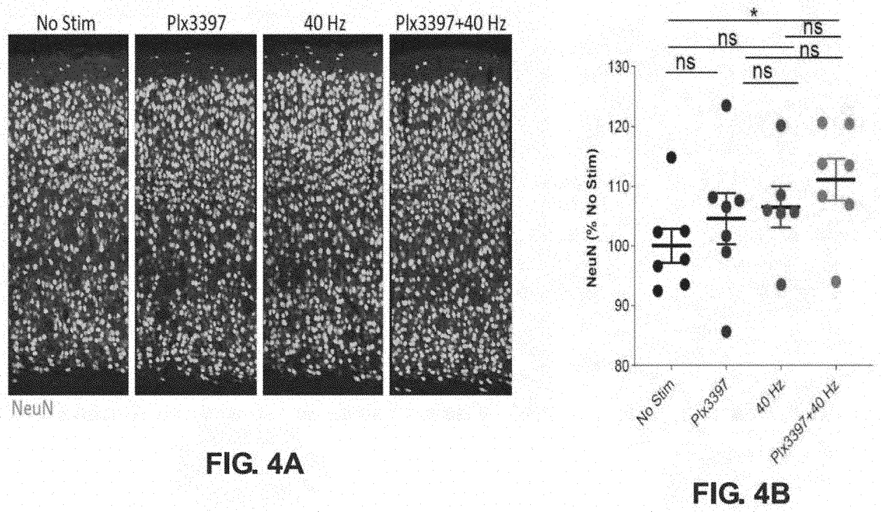

Loss of synapses and neurons are closely associated with higher neuroinflammatory response and cognitive decline in AD. Therefore, synaptic markers in the visual cortex of Plx3397 and/or GENUS treated mice were evaluated. Plx3397, GENUS, and Plx3397+GENUS treated mice exhibited a significant increase in vGAT (Synaptic Systems, #131 013) positive synaptic puncta (ANOVA, F (3,29)=8.831, P=0.0003) (FIGS. 2A, 2B). A stronger increase in vGAT puncta in Plx3397+GENUS treated mice compared to Plx3397 and GENUS mice was also observed (FIG. 2B). In addition, 5×FAD mice treated with Plx3397+GENUS showed significantly higher synaptophysin (Sigma, #S5768) signals in western blots from visual cortex tissue (ANOVA, F (3,22)=7.216, P=0.0015) (FIGS. 3A-3B). Previous studies showed a neuronal loss in layer 5 cortex in 11-month-old 5×FAD mice. Thus, whether these treatments had any effect on neuronal density by immunohistochemical analysis of neuronal marker NeuN was examined (Synaptic Systems, #266 004) positive cells (FIG. 4A). It was found that 5×FAD mice treated with Plx3397+GENUS showed significantly higher neuronal density in the visual cortex (FIG. 4B). These results suggest that microglia reduction together with GENUS can improve neuronal and synaptic density in 5×FAD mice.

Combined Administration of CSF1 Inhibitor and GENUS Reduces Neuroinflammatory Markers

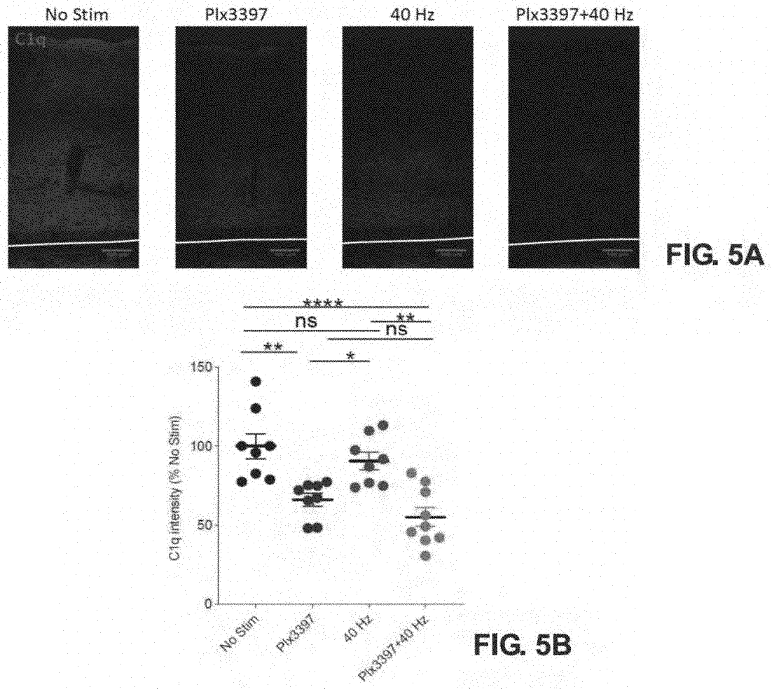

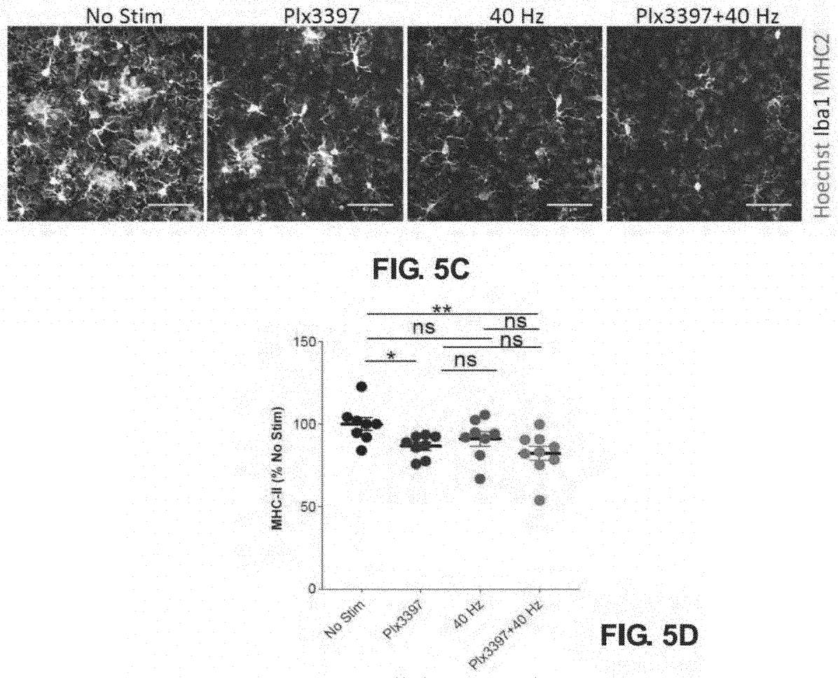

Elevated expression of C1q is observed in both human AD and mouse models of AD, and further such an elevated expression is closely associated with the elimination of synapses in microglia. In addition, MHC2 expression is increased in the AD brain. Therefore, to test whether these treatments, that improved synaptic density, had any effect on these neuroinflammatory markers, C1q (Abcam, #ab182451) and MHC2 (EMD Millipore, #MABF33) levels (FIGS. 5A-5D) were examined. It was observed that combined treatment of Plx3397 and GENUS significantly reduced C1q (ANOVA, F (3,29)=12.07, P<0.0001) (FIG. 5B) and MHC2 levels (ANOVA, F (3,29)=3.861, P=0.0194) (FIG. 5D) in the visual cortex, suggesting that the improved synaptic density associates with reduced inflammatory markers after Plx3397+GENUS treatment in the 5×FAD mice.

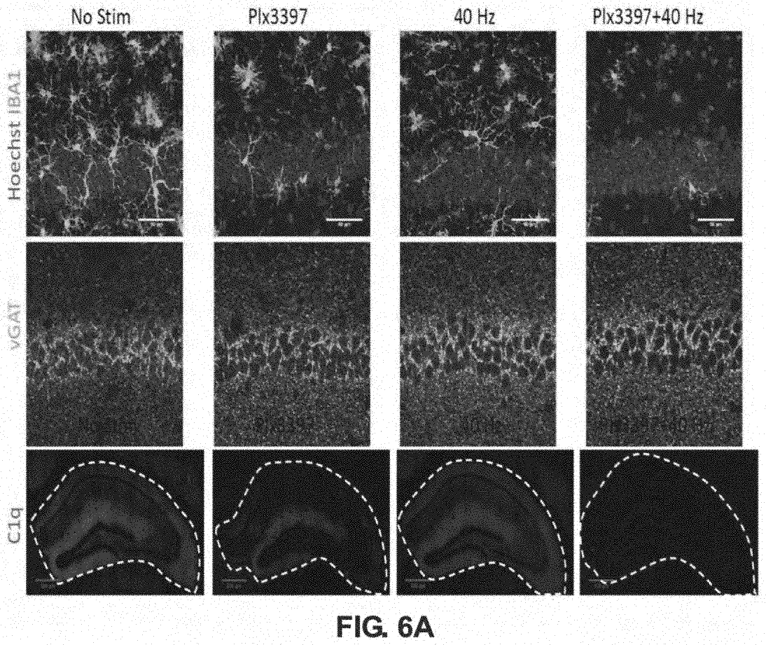

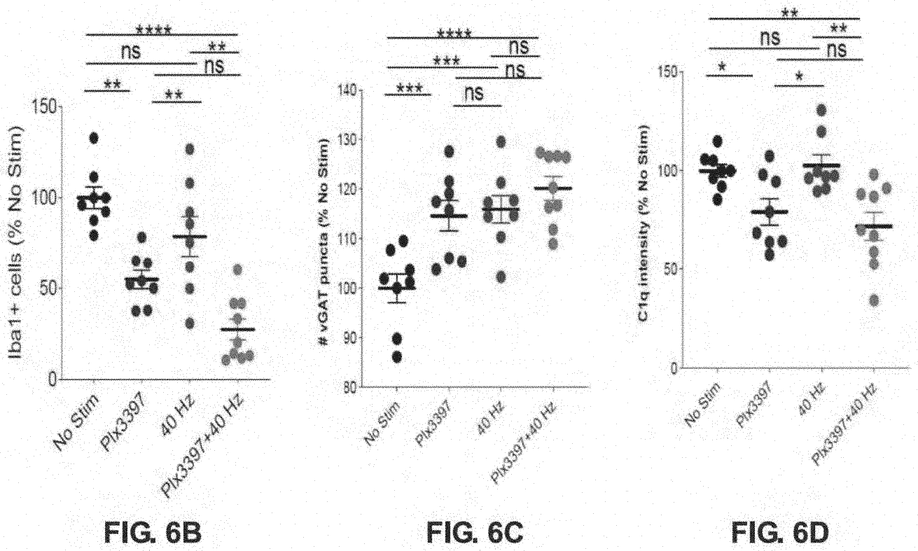

The effect of the treatments in the hippocampus were next evaluated. Consistent with the results obtained in the visual cortex, Plx3397, and Plx3397+GENUS treatment groups showed a significant reduction in IBA1+ positive microglia in the CA1 region of the hippocampus (ANOVA, F (3,29)=19.1, P<0.0001) (FIGS. 6A, 6B). Further, Plx3397+GENUS treatment significantly increased vGAT synaptic puncta (ANOVA, F (3,29)=10.09, P=0.0001) (FIG. 6C) while reducing C1q signal (ANOVA, F (3,29)=7.015, P=0.0011) (FIG. 6D) compared to no treatment in the hippocampus in 5×FAD mice.

Combined Administration of CSF1 Inhibitor and GENUS Improves Synaptic Density while Reducing Inflammatory Markers in the CK-p25 Mice.

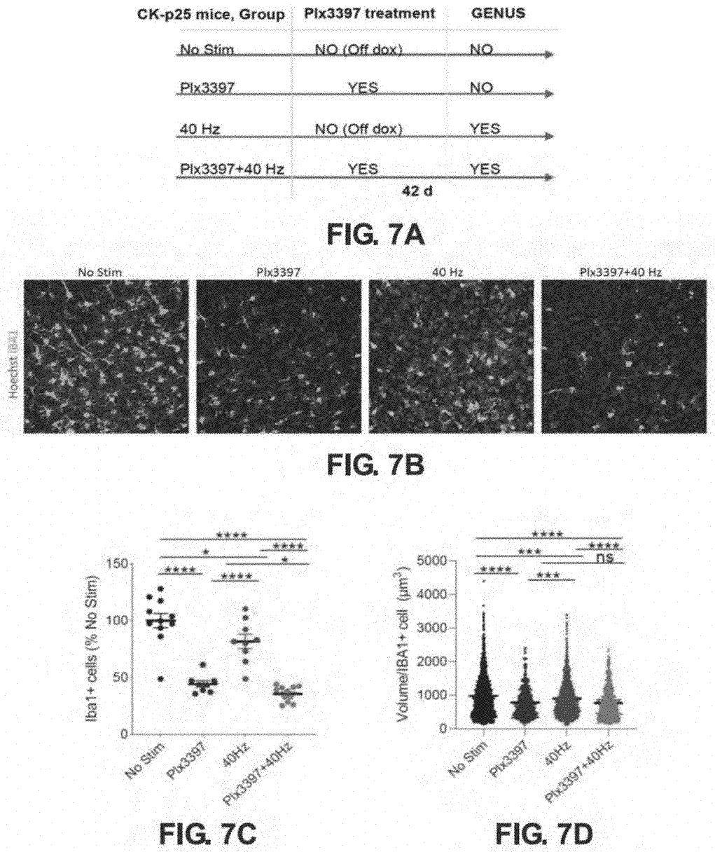

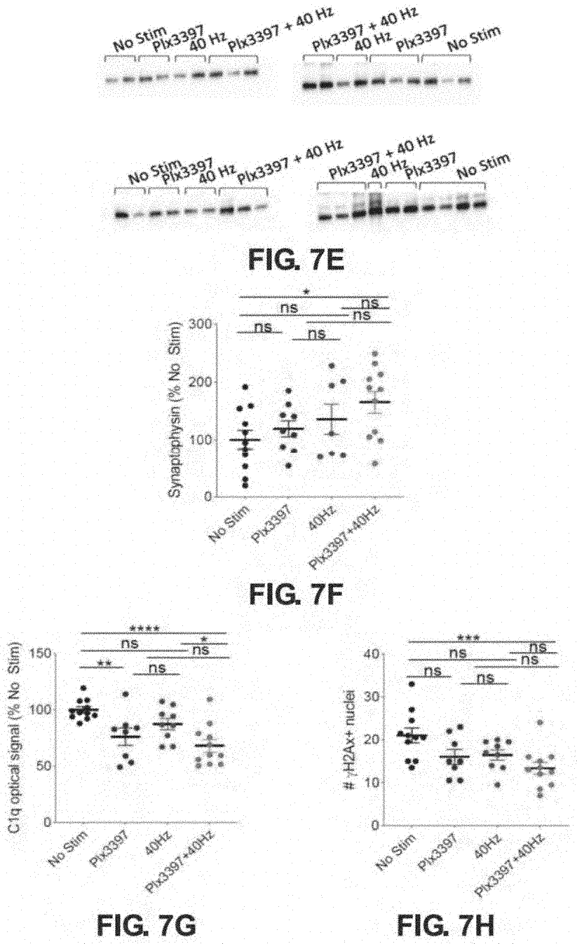

Whether the neuroprotective effect is broader and can be replicated in other mouse models of neurodegeneration was evaluated. The CK-p25 mice, transgenic mice that overexpress CDK5 activator p25 in excitatory neurons, were subjected to these treatments. CK-p25 mice, which was raised in doxycycline containing food, was given either normal rodent chow (containing no doxycycline) or Plx3397 chow, and the mice simultaneously underwent no sensory stimulation or GENUS (FIG. 7A). After 42 days of treatment, neuroprotective factors were evaluated. Plx3397, GENUS and Plx3397+GENUS treatments reduced microglia in the visual cortex in CK-p25 mice (ANOVA F (3,35)=40.93, P<0.0001) (FIGS. 7B-7D), consistent with the results observed in 5×FAD mice and recent findings. Further, it was observed a synergistic effect in Plx3397+GENUS treated mice, which had significantly fewer IBA1+ cells than mice receiving either treatment alone (FIG. 7C). Plx3397, GENUS and Plx3397+GENUS treatments also resulted in lower volume of microglia (ANOVA F (3,35)=27.41, P<0.0001) (FIG. 7D). Examination of synaptic and inflammatory markers revealed that Plx3397+GENUS treatment increased expression of synaptophysin (ANOVA F (3,34)=2.55, P=0.04) while reducing C1q (ANOVA F (3,35)=7.835, P=0.0004) in the visual cortex (FIGS. 7E-7G). In addition, γH2Ax, a known marker for DNA damage and is highly increased in CK-p25 mice, was significantly reduced after Plx3397+GENUS treatment (ANOVA F (3,35)=4.825, P=0.0065) (FIG. 7H). Together, these results suggest that Plx3397+GENUS treatment improves protective neuronal and/or synaptic markers while reducing pathological neuroinflammatory markers in two distinct mouse models of neurodegeneration (5×FAD and CK-p25).

Combined Administration of CSF1 Inhibitor and GENUS Induces Gene Expression Changes in Microglia

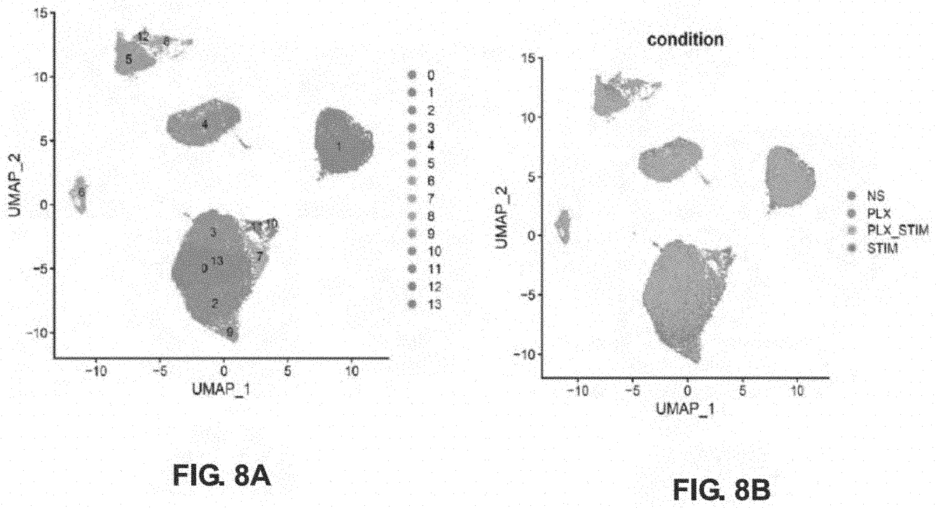

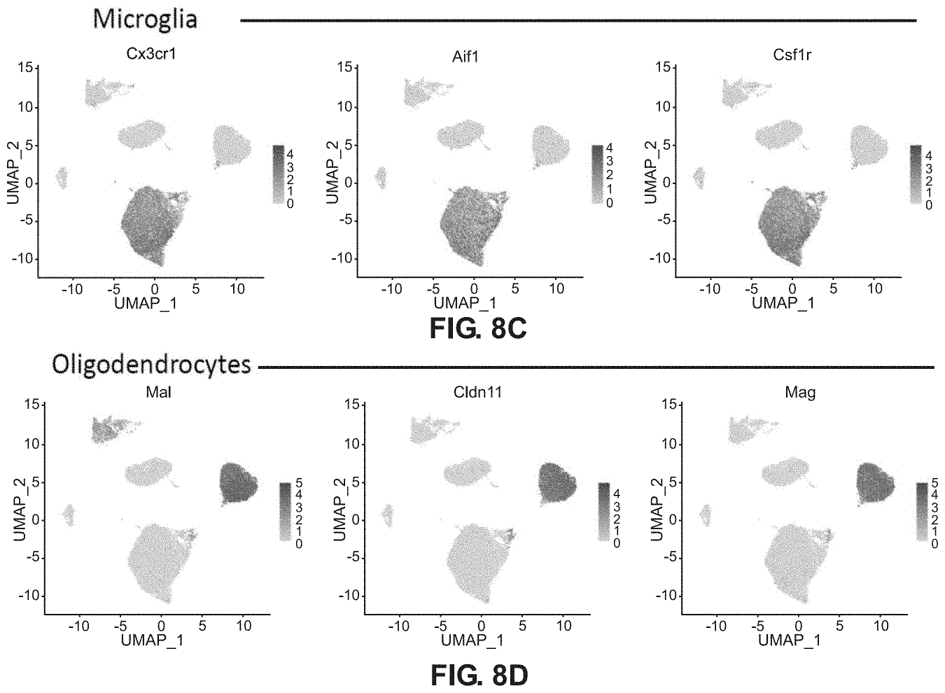

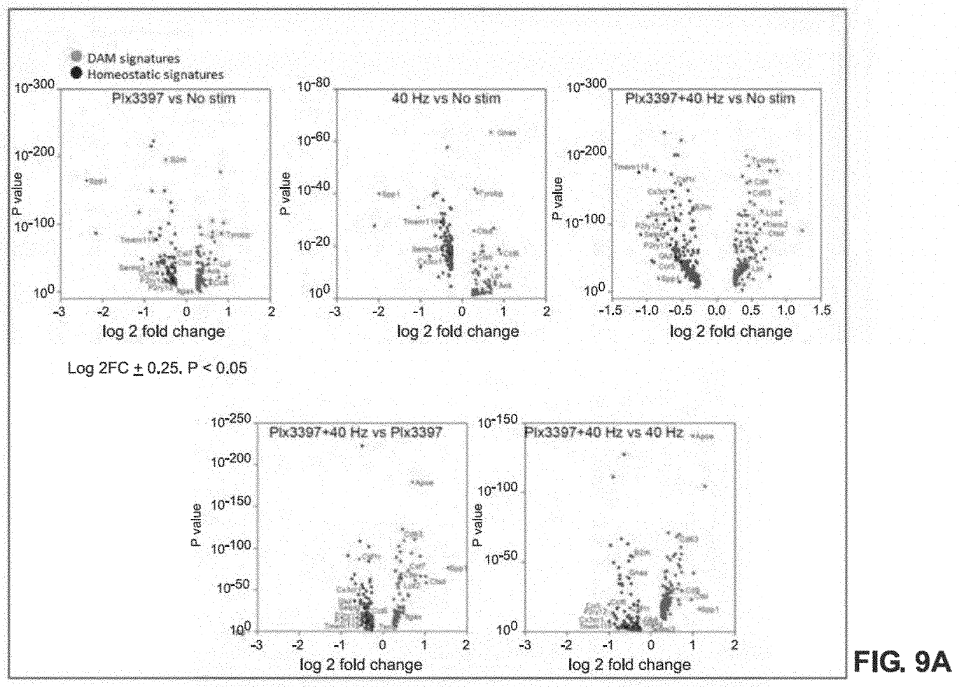

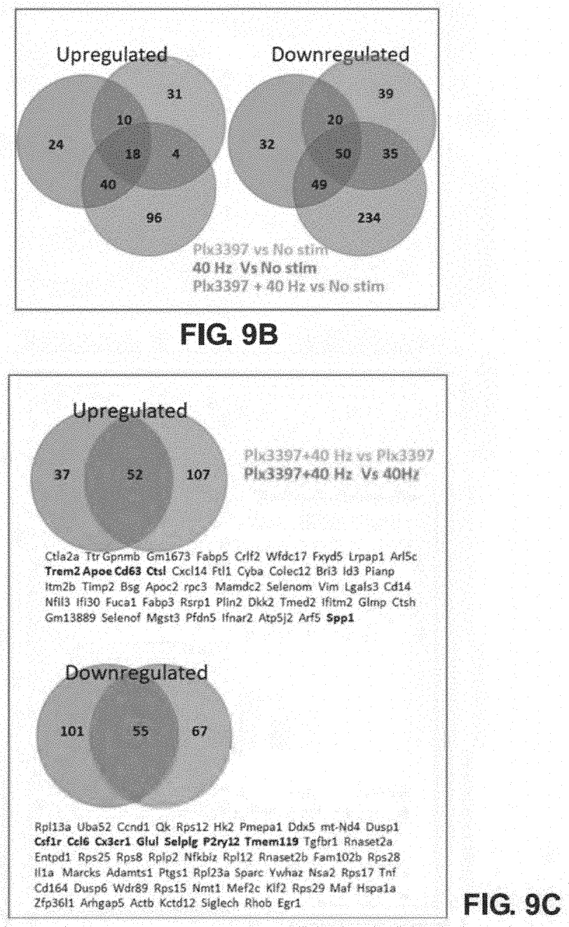

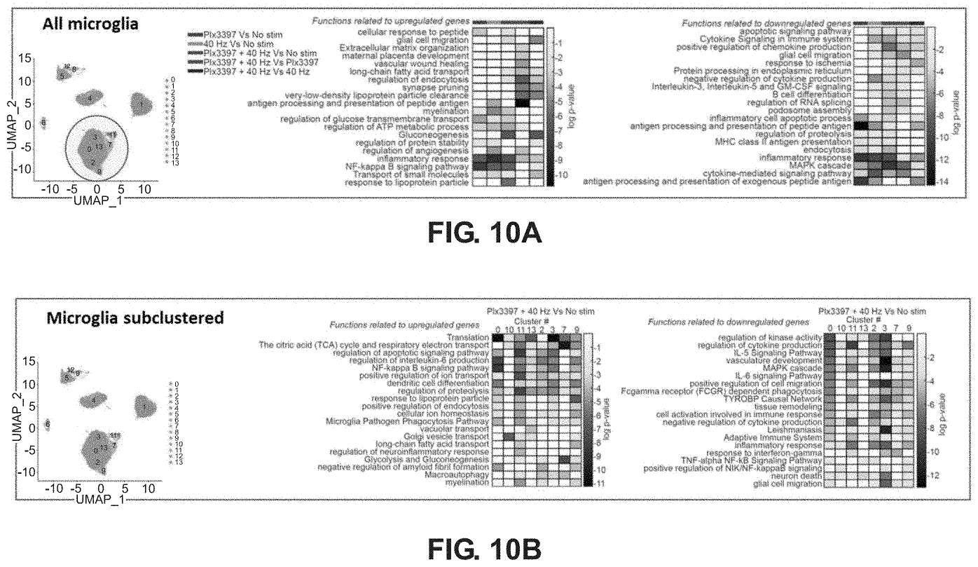

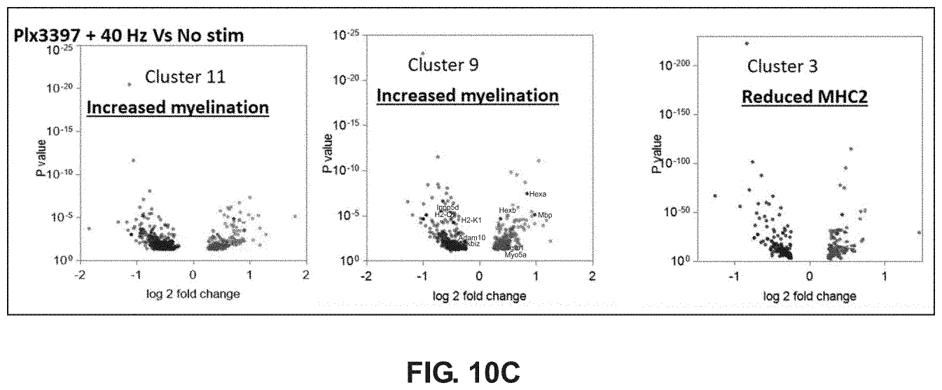

Gene expression changes in microglia are strongly associated with AD pathogenesis. Thus, the effect of these treatments on gene expression using single-cell RNA sequencing was investigates (10× Genomics, #Chromium Next GEM Single Cell 3′ Kit v3.1, 16 rxns PN-1000268). Clustering of cells based on the marker genes revealed a good representation of microglia (Cx3cr1, Aif1, and Csflr) and oligodendrocyte (Mal, Mag, and Cldn11) cell populations in the dataset (FIGS. 8A-8D). The gene expression changes between treatment conditions in the microglia cluster were examined. It was observed that Plx3397, GENUS, and Plx3397+GENUS treatments upregulated genes related to Tyrobp-trem2-Apoe pathway in microglia (FIGS. 9A-9C). Overall, gene enrichment analysis revealed that these treatments increased the clearance of low-density lipoprotein, extracellular matrix organization, vascular wound healing, regulation of protein stability, and the organization of vesicles (FIG. 10A). Previous studies showed that microgliosis is associated with the increased expression of genes related to MHC-II antigen presentation and inflammatory response in AD. It was observed that genes related to these processes in microglia were downregulated after Plx3397+GENUS treatment in 5×FAD mice (FIGS. 10A, 10B). Further, due to the depth of the gene expression analysis sub-clustering of microglia was performed. This revealed several distinct clusters of microglia, after the treatment with two sub-clusters of microglia showing genes related to myelination upregulated (Mbp, Igf1, Tgf1b, Hexa) and a sub-cluster showing reduced NMC-II genes (Cd74, Hz-Aa, H2-Ab1, H2-Eb1) (FIGS. 10B, 10C). Together, these results suggest that microglia reduction combined with GENUS induces unique gene expression changes associated with the neuroprotective effects.

Combined Administration of CSF1 Inhibitor and GENUS Induces Gene Expression Changes in Oligodendrocytes

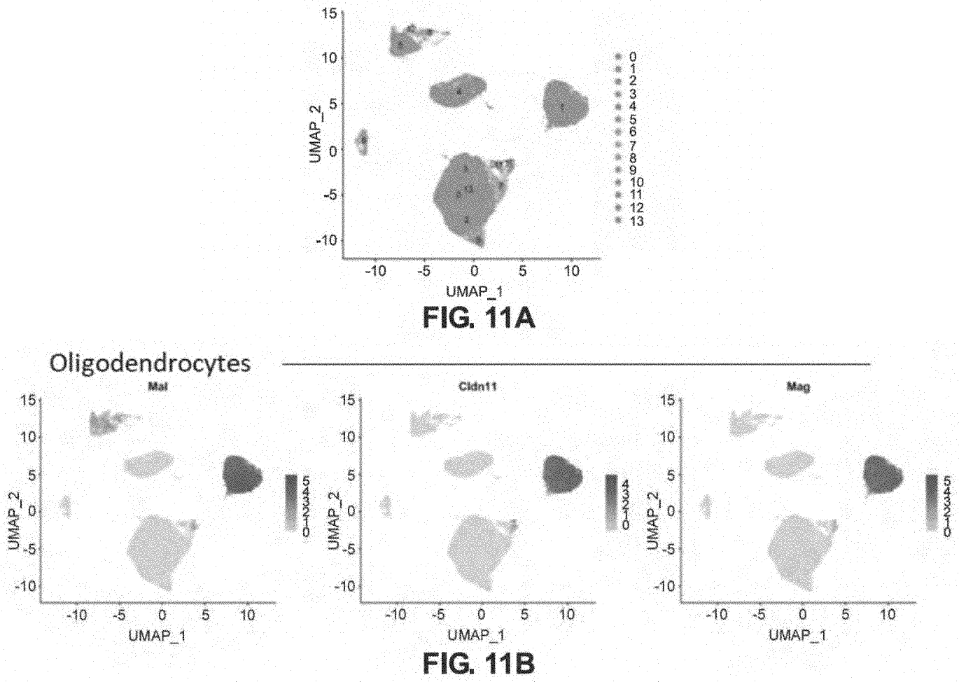

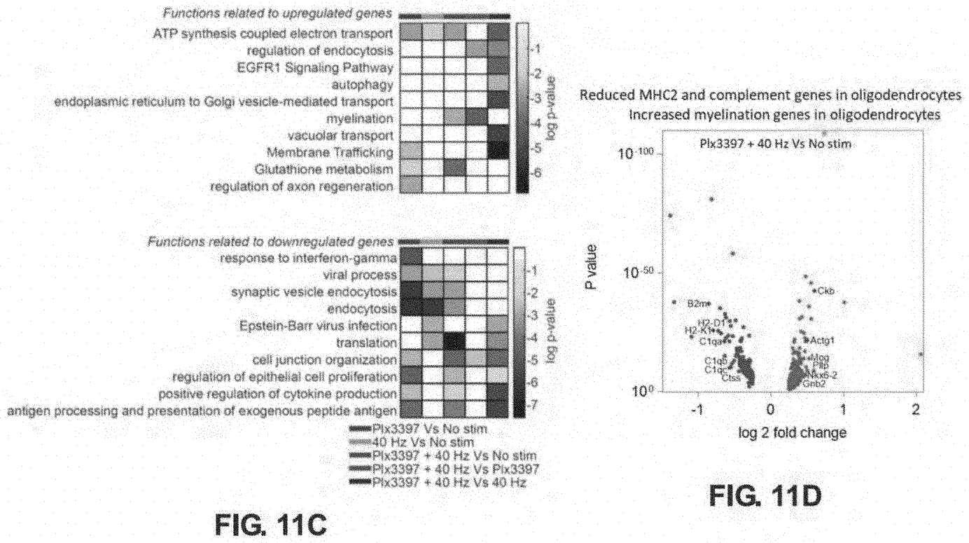

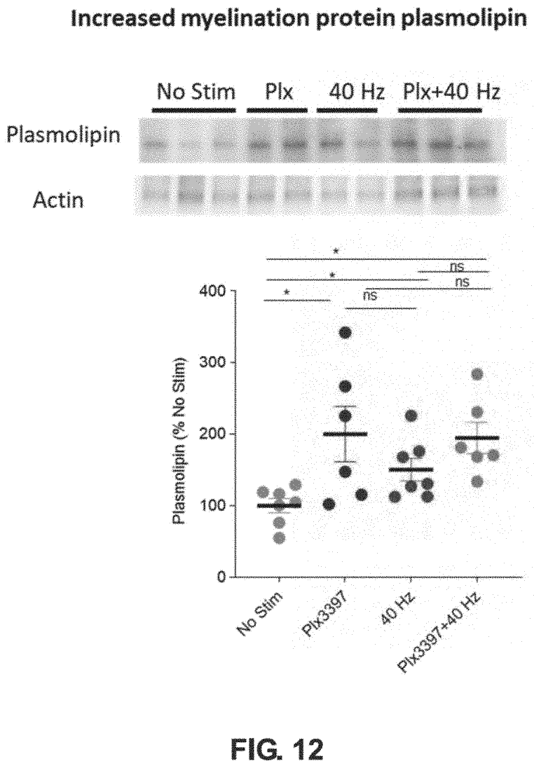

The gene expression changes between treatment conditions in the oligodendrocytes cluster were examined. Previous studies showed that MHC-II and complement pathway is associated with reduced myelination. Combined treatment upregulated genes related to myelination (Mog, Pllp, Nkx6-2, Gnb2), whereas it reduced genes related to MHC-II (H2-K1, H2-D1) and complement (C1a, C1b, C1q) (FIGS. 11A-11D). Further validation with immunoblot revealed that myelination protein plasmolipin was upregulated after the combined treatment (FIG. 12). Together, these results suggest that Plx3397+GENUS treatment improves myelination while reducing pathological neuroinflammatory markers such as MHC-II and complement pathway genes.

Combined Administration of CSF1 Inhibitor and GENUS Induces Gene Expression Changes in Neurons

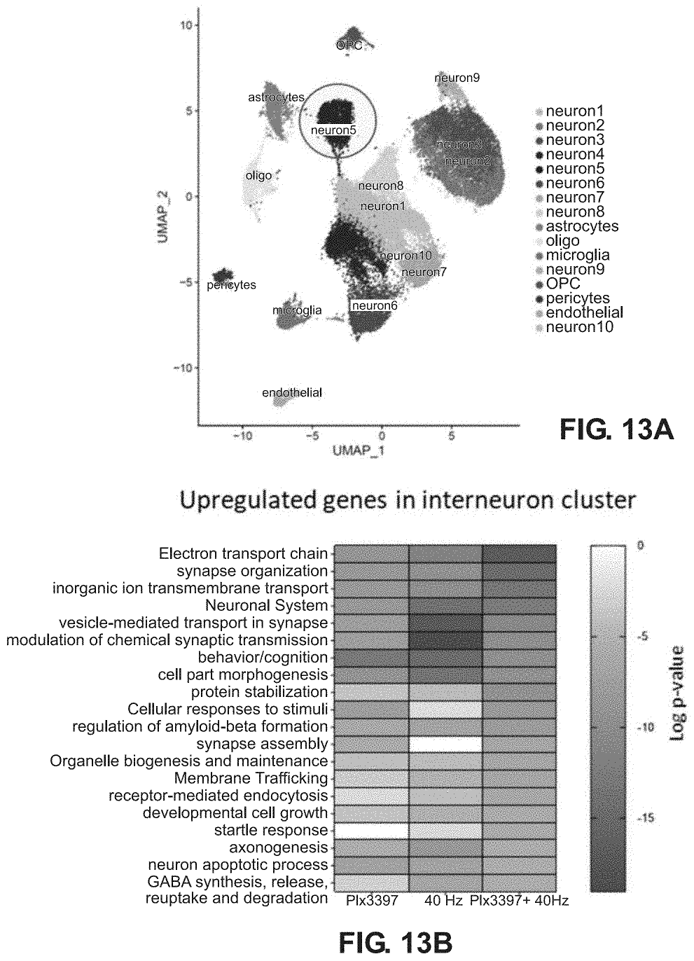

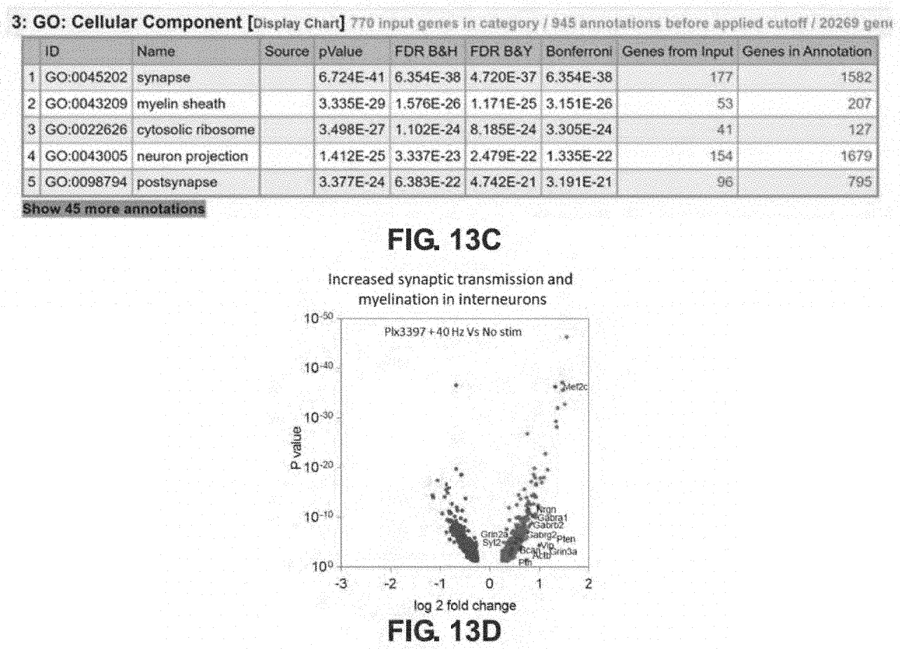

As neurons were not represented from the single-cell RNA-sequencing (FIGS. 8A-12), to study the effect of these treatments on gene expression in neurons, single nucleus RNA-sequencing was performed (10× Genomics, #Chromium Next GEM Kit v3.1, 16 rxns PN-1000268). Clustering of cells based on the marker genes revealed a good representation of all major neural cell types including excitatory neurons, interneurons and other glial cell populations in the dataset (FIG. 13A). The gene expression changes between treatment conditions in the interneurons cluster were examined. Learning and memory, synapse assembly and organization, membrane trafficking and intracellular transport related genes were all up-regulated (FIG. 13B). In addition, the majority of the upregulated genes are also involved in myelination (Pten, Actb), and excitation and inhibition balance (Mef2c) (FIGS. 13C, 13D).

Combined Administration of CSF1 Inhibitor and GENUS Induces Synaptic Gene Expressions

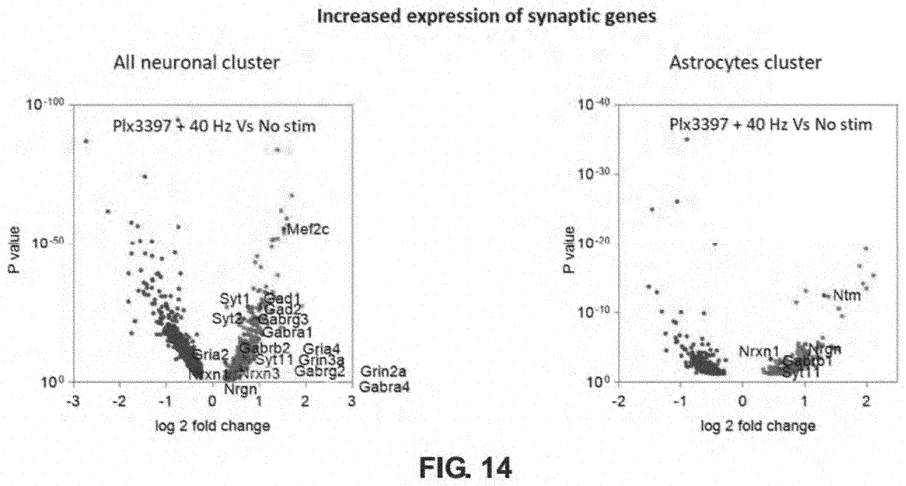

Neurons and astrocytes together form tripartite synapses. Unbiased RNA-sequencing revealed that the CSF1R inhibitor+GENUS combined treatment significantly elevated the expression of many synaptic genes in both neurons and astrocytes. These genes include NMDA-receptors (Grin2a, Grin3a), AMPA-receptors (Gria2, Gria4), GABA-receptors (Gabral, Gabrb2, Gabrg3) and general synaptic genes (Nrxnl, Nrgn, Syt1, Syt2) in neurons (FIG. 14). In astrocytes, CSF1R inhibitor+GENUS combined treatment increased the expression of Nrx1, Syt11, Nrgn, Ntm and Gabrb1. Together, these results suggest that CSF1R inhibitor+GENUS combined treatment increased overall expression of synaptic genes and possibly improved the communication between neurons and astrocytes (FIG. 14).

Combined Administration of CSF1 Inhibitor and GENUS Enhances Phase Locking of Neurons to Gamma Oscillations In Vivo

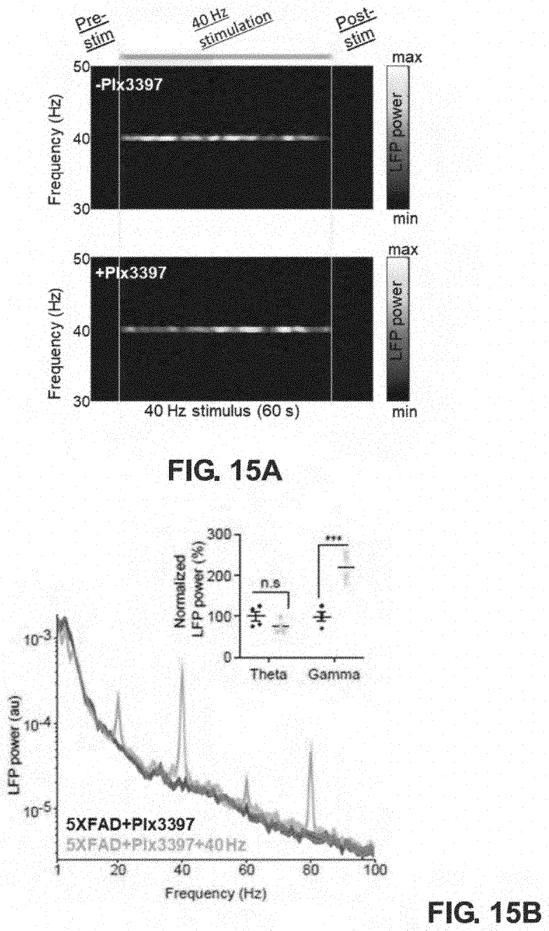

Next, it was aimed to understand how these treatments impacted the LFP oscillations and neuronal action potentials. First, in vivo awake animal electrophysiology was performed using high-density linear probes and verified whether 5×FAD mice with reduced microglia by Plx3397 treatment can entrain 40 Hz sensory stimulation. It was observed that plx3397 treated 5×FAD mice can indeed entrain 40 Hz (FIGS. 15A, 15B). Further, gamma response latency is comparable between untreated and Plx3397 treated mice, suggesting that the microglia reduction did not impact sensory response time in the cortex. At the group level gamma stimulation increased gamma but not theta power as expected based on previous findings (2 W ANOVA, groups x frequency, F (1,6)=32.03, P=0.0013) (FIGS. 15A, 15B).

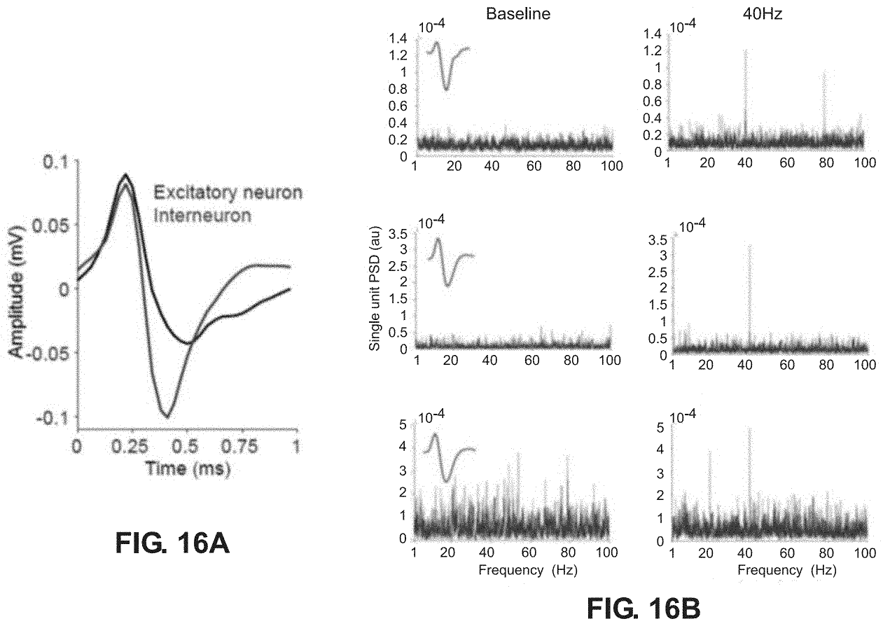

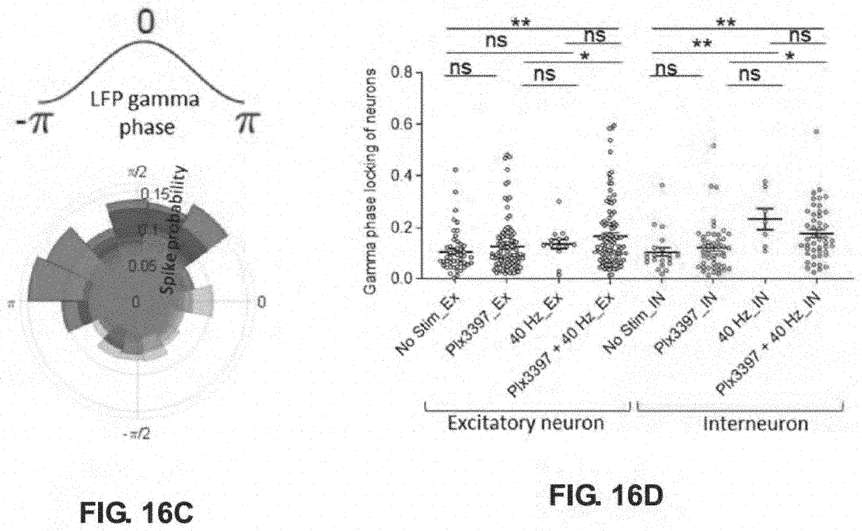

Next, principal component analyses was performed using action potential properties, isolated single units, and further separated them into excitatory and interneurons (FIG. 15A). Interneurons in Plx3397+GENUS treated mice exhibit clear gamma entrainment with an LFP phase preference around descending phase (FIGS. 16B, 16C). Importantly, phase locking of both excitatory neurons (ANOVA, F (3, 257)=4.006, P=0.0082) and interneurons (ANOVA, F (3, 117)=5.393, P=0.0016) with LFP gamma was significantly enhanced after Plx3397+GENUS treatment in 5×FAD mice (FIG. 16D). Together, these findings suggest that microglia reduction in combination with daily GENUS improves the relationship between an ensemble of neurons as evaluated by enhanced phase locking of individual neurons with population activity reflected by LFP in 5×FAD mice.

Combined Administration of CSF1 Inhibitor and GENUS Increases the Perineuronal Net of Neurons

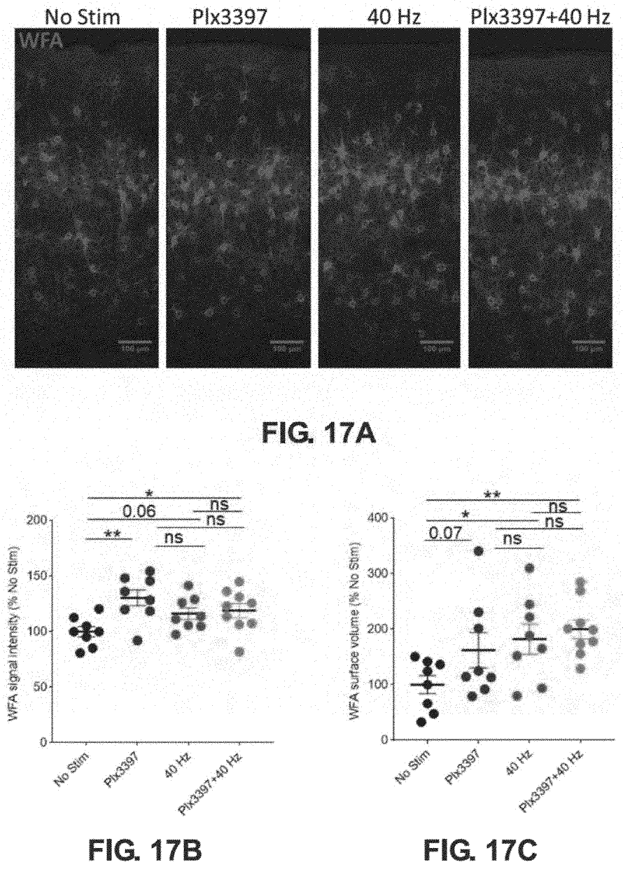

Perineuronal nets (PNN) of neurons are necessary for neuronal integrity and circuit plasticity. Loss of PNN is shown to occur in AD. The gene expression and electrophysiological analyses suggested a strong effect of these treatments on cortical circuit. Specifically, several commonly upregulated genes (e.g. Mamdc2, Itm2b) after Plx3397+GENUS treatment is implicated in an extracellular matrix organization. Further, Plx3397+GENUS treatment improved phase locking of neurons. Therefore, the effect of the treatment on PNN was examined. Staining of Wisteria floribunda lectin (WFA, WFL; Vector Biolabs, #B-1355-2), the most commonly used method to label PNN, indeed revealed that the Plx3397, GENUS, and Plx3397+GENUS increased overall signal intensity (ANOVA, F (3,29)=4.307, P=0.0125) and PNN coverage (ANOVA, F (3,29)=3.432, P=0.0299) in the visual cortex in 5×FAD mice (FIGS. 17A-17C).

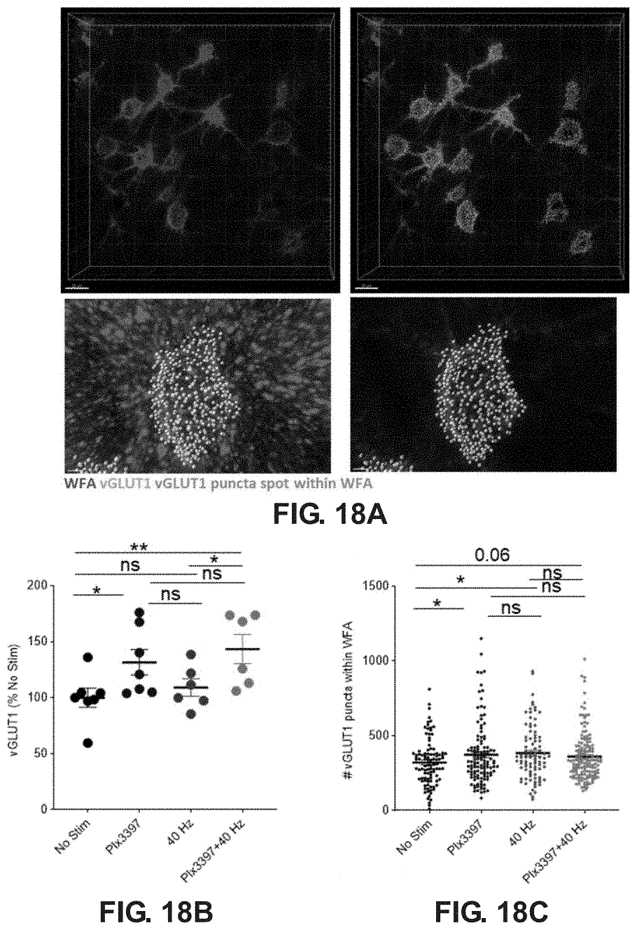

It was reasoned that the increased WFA coverage and enhanced phase locking of neurons after these treatments are related. Thus, whether synaptic markers are also enhanced within WFA carrying neurons, which are shown to significantly regulate plasticity and circuit architecture, was tested. WFA has been predominantly observed around interneurons, specifically PV interneurons. vGLUT1 (Synaptic Systems, #1135 302), an excitatory synaptic marker, was labeled with WFA. It was observed that the overall vGLUT1 signal was higher after Plx3397+GENUS treatment in mice (ANOVA, F (3,22)=3,686, P=0.0273) (FIGS. 18A, 18B). WFA was 3D rendered and surface created and examined (vGLUT1) within WFA, and observed increased vGLUT1 within WFA (Nested ANOVA, F (3,29)=3.377, P=0.0493) (FIGS. 18A, 18C). Together, these results suggest that Plx3397+GENUS treatment improves extracellular matrix with more synaptic density within PNN in 5×FAD mice.

Combined Administration of CSF1 Inhibitor and GENUS Enhances Novel Object Recognition Memory.

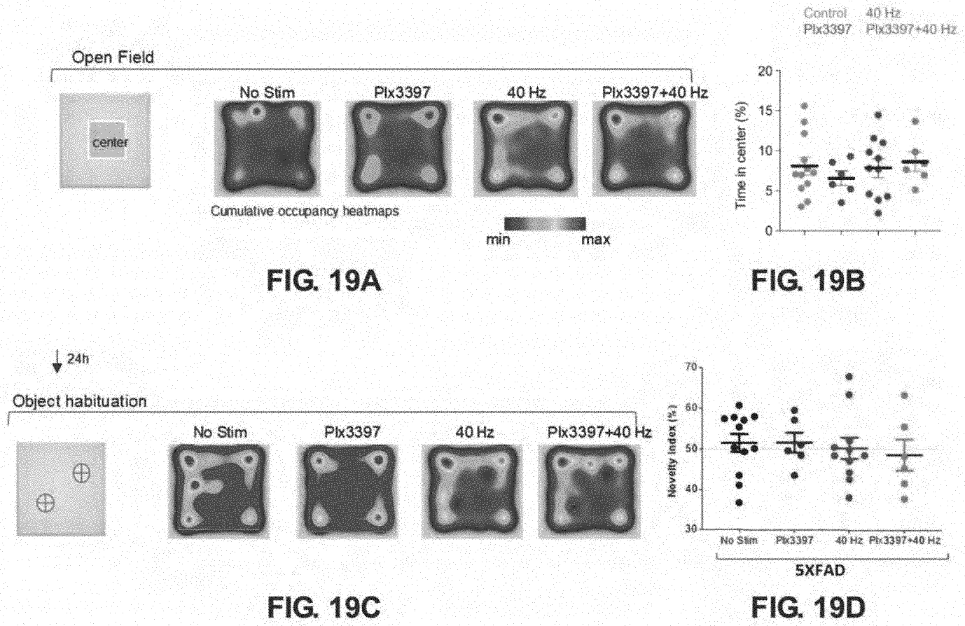

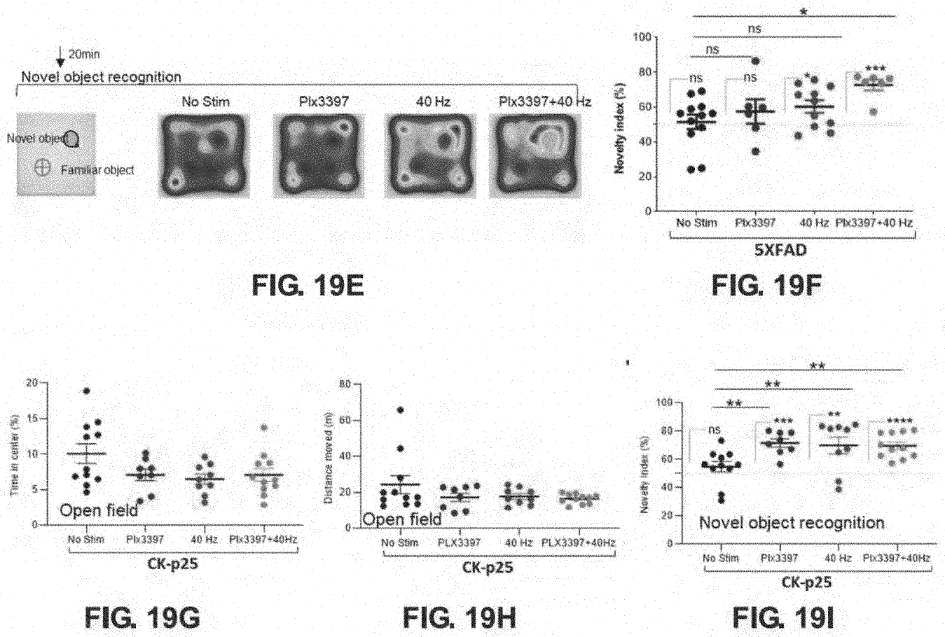

Given the neuroprotective effect of Plx3397+GENUS treatment, whether this treatment also impacted learning and memory was evaluated. Mice were tested in an open field (OF) and assessed for changes in anxiety and activity levels, followed by a novel object recognition (NOR) test of memory. 10-month-1d 5×FAD mice were treated with Plx3397. Following 20 days administration of Plx3397 chow, untreated- and Plx3397 treated 5×FAD mice (which continued PLX administration) were subjected to 30 days of daily GENUS. Mice were tested in OF and NOR during the last week of these treatments. None of the treatments had any effect on the time spent in the center of the OF arena compared to control-treated 5×FAD mice (ANOVA F (3,31)=0.384, P=0.764) (FIGS. 19A, 19B), suggesting no changes in anxiety level. Consistently, these treatments did not overtly affect exploratory behavior during habituation for the NOR (ANOVA F (3,31)=0.2198, P=0.8819) (FIGS. 19C, 19D). In NOR, GENUS, and Plx3397+GENUS treated 5×FAD mice but not control-treated and Plx3397 treated mice showed an increased preference for the novel object compared to chance level (50%) (FIGS. 19E, 19F). Further, Plx3397+GENUS treatment significantly improved NOR memory compared to no treatment (ANOVA F (3,31)=3.456, P=0.0282). Next, CK-p25 mice were treated for 42 days and assessed behavioral performance. No significant difference in the time spent in the center of the arena (ANOVA F (3,35)=2.563, P=0.070), and total distance traveled (ANOVA F (3,35)=1.516, P=0.227) was found between any groups in OF test (FIGS. 19G, 19H). It was observed that Plx3397, GENUS, and Plx3397+GENUS treatments significantly improved novel object recognition memory in the NOR test (ANOVA F (3,35)=4.224, P=0.0119) (FIG. 19I). Overall, these data suggest that Plx3397+GENUS can improve novel object recognition memory in multiple mouse models of neurodegeneration.

### DISCUSSION

Data presented herein is consistent with the view that; (a) Plx3397 treatment reduces microglia, microglia-mediated neuroinflammation, and synaptic elimination, and (b) the preserved synapses are then reorganized & strengthened by repeated GENUS. This view is supported by evidence at multiple levels of analysis. Specifically, Plx3397+GENUS treatment (a) reduced inflammatory markers, which are closely associated with increased excitatory and inhibitory synaptic markers, (b) increased extracellular matrix reorganizing genes in microglia, which closely associated with increased perineuronal nets, and (c) neurons are strongly coupled with gamma oscillations. These protective changes are associated with the improvements in recognition memory in AD mice. In conclusion, these findings suggest that anti-inflammatory drugs can be combined with non-invasive gamma stimulation to offer neuroprotection and cognition in AD.

Methods

Animal Models

All the experiments were approved by the Committee for Animal Care of the Division of Comparative Medicine at the Massachusetts Institute of Technology (MIT), and carried out at MIT. Tg(Camk2a-tTA), and Tg(APPSwFlLon, PSEN1*M146L*L286V) were obtained from the Jackson laboratory. Tg(tetO-CDK5R1/GFP) was generated.

GENUS Stimulation

Light flicker stimulation was delivered as previously described. Mice were transported from the holding room to the flicker room, located on adjacent floors of the same building. Mice were habituated under dim light for 1 hour before the start of the experiment, and then introduced to the test cage (similar to the home cage, except without bedding and three of its sides covered with black sheeting). All GENUS protocols were administered on a daily basis for 1 h/d for the number of days as specified. Mice were allowed to freely move inside the cage but did not have access to food or water during the 1 hour light flicker. An array of light-emitting diodes (LEDs) was present on the open side of the cage and was driven to flicker at a frequency of 40 Hz with a square wave current pattern using an Arduino system. The luminescence intensity of light that covered inside the total area of GENUS stimulation cage varied from ˜200-1000 lux as measured from the back and front of the cage (mice were free to move in the cage). After 1 h of light flicker exposure, mice were returned to their home cage and allowed to rest for a further 30 min before being transported back to the holding room. No-stimulation mice underwent the same transport and were exposed to similar cages with similar food and water restriction in the same room, but experienced normal room light (of similar lux as 40 Hz stimulation) for the 1 h duration. Experimenters who stimulated the mice were male.

Open Field (OF) and Novel Object Recognition (NOR) Test

For OF, mice were introduced into an open field box (dimensions: length=460 mm, width=460 mm and height=400 mm; TSE-Systems) and were tracked using Noldus (Ethovision) for 12 min, with time spent in the center and peripheral area of the arena measured. NOR occurred on the following day, when mice were re-introduced into the same open field box which now additionally contained two identical novel objects and were allowed to explore the objects for 7 min (novel object habituation). Mice were then placed back in their home cages for 20 min after the last exploration. They were then returned to the same arena, with one of the two objects replaced with a new object. Mouse behavior was monitored for 7 min. Time spent exploring both the familiar and novel objects was recorded using Noldus and computed offline. Percentage of novelty preference index was calculated as follows: time exploring novel object (Nt) divided by total time exploring novel and familiar (Ft) objects and presented in %−{[Nt/Nt+Ft]*100}.

Immunohistochemistry

Mice were transcardially perfused with 40 mL of ice-cold phosphate-buffered saline (PBS) followed by 40 mL of 4% paraformaldehyde (PFA; Electron Microscopy Sciences, Cat #15714-S) in PBS. Brains were removed and post-fixed in 4% PFA overnight at 4° C. and transferred to PBS prior to sectioning. Brains were mounted on a vibratome stage (Leica VT 1000S) using superglue and sliced into 40 mm sections. Slices were subsequently washed with PBS and blocked using 5% normal donkey serum prepared in PBS containing 0.3% Triton X-100 (PBST) for 2 hours at room temperature. Blocking buffer was aspirated out and the slices were incubated with the appropriate primary antibody (prepared in fresh blocking buffer) overnight at 4° C. on a shaker. Slices then were washed three times (10 min each) with the blocking buffer and then incubated with the Alexa Fluor 488, 555, 594 or 647 conjugated secondary antibodies for 2 hours at room temperature. Following three washes (15 min each) with blocking buffer and one final wash with PBS (10 min), slices were mounted with fluromount-G (Electron microscopic Sciences). The following combination of secondary antibodies were used: (1) Alexa Fluor 488, 594 and 647, (2) Alexa Fluor 555 and 647, (3) Alexa Fluor 594 and 647, or (4) Alexa Fluor 488 and 647.

Images were acquired using either LSM 710 or LSM 880 confocal microscopes (Zeiss) with 10×, 20×, or 40× objectives at identical settings for all conditions. Images were quantified using Imarisx64 9.3 (Bitplane, Switzerland). For each experimental condition, two coronal sections per mouse from the indicated number of animals were used. The averaged values from the two to four images per mouse were used for quantification. The experimenter blinded to the treatment conditions performed all the image processing and quantification.

NeuN and gH2Ax positive cell: All images were acquired in Z stacks—10 per image (step of 2 μm) and were quantified. The spot-count inbuilt function in multi-point tool in Imarisx64 9.3 was used to count cells automatically.

vGAT and vGLUT1 puncta: LSM 710, with a 40× objective, was used to acquire the images. The entire 40 m thickness of the slices was acquired in Z stacks—80 per image (step of 0.5 μm). The spot-count inbuilt function in Imarisx64 9.3 was used to count cells automatically.

C1q and MHC2 signal intensity: Using an LSM 710 with a 20× or 40× objectives, z stacks of the entire slice thickness 40 mm (40 images from each field) were acquired. The signal intensity was measured.

Microglia: Iba1 immunoreactive cells were considered microglia. Using an LSM 710 or LSM 880 with a 10× (for Iba1+ cell counts) or 40× (for morphological analysis) objective z stacks of the entire slice thickness 40 m with 0.5 m step size were acquired. Imaris was used for 3D rendering of images to quantify the total volume of microglia.

Western Blotting

The visual cortex was dissected out and snap-frozen in liquid nitrogen and stored in an −80° C. freezer until processing. Samples were homogenized using a glass homogenizer with RIPA (50 mM Tris HCl pH 8.0, 150 mM NaCl, 1% NP-40, 0.5% sodium deoxycholate, 0.1% SDS) buffer which contains protease and phosphatase inhibitor. The concentration of proteins in samples were quantified using a Bio-Rad protein assay. Equal concentrations of proteins were prepared and added with SDS—sample buffer. Ten mg of protein was loaded onto 4-20% polyacrylamide gels and electrophoresed. Protein was transferred from acrylamide gels to nitrocellulose membranes for 12 min (Semi-dry system, Bio-Rad). Membranes were blocked using BSA (5% w/v) diluted in TBS containing 0.1% Tween-20 (TBSTw), then incubated in primary antibodies overnight at 4° C. The following day, they were washed three times with TBSTw and incubated with horseradish peroxidase-linked secondary antibodies (GE Healthcare) at room temperature for 60 min. After three further washes with TBSTw, membranes were treated with chemiluminescence substrates and the blots were visualized (Chem doc, Bio-Rad). Signal intensities were quantified using ImageJ 1.46q and normalized to values of loading control.

In Vivo Electrophysiology

Mice were anaesthetized with isoflurane, restrained in a stereotactic apparatus and craniotomies were made exposing the visual cortex (AP: −3.2 & ML: +2.5). Linear probes (Neuronexus) Probes were implanted and slowly lowered to the target depth. The reference electrode was targeted to the white matter tract above the hippocampus. Mice were allowed to recover for a period of 4 days.

Following a 2-3-day habitation period for the recording, recordings commenced with the animal allowed to move freely in their home cages. Data were acquired using Neuralynx SX system (Neuralynx, Bozeman, Mont., USA) and signals were sampled at 32,000 Hz. The position of animals was tracked using red light-emitting diodes affixed to the probes. At the conclusion of the experiment, mice underwent terminal anesthesia and electrode positions were marked by electrolytic lesioning of brain tissue with 50 mA current for 10 s through each electrode individually, to confirm their anatomical location.

Spikes

Single units were manually isolated by drawing cluster boundaries around the 3D projection of the recorded spikes, presented in SpikeSort3D software (Neuralynx). Cells were considered pyramidal neurons if the mean spike width exceeded 220 ms and had a complex spike index (CSI)≥5.

Data Analyses

LFPs were first filtered to the Nyquist frequency of the target sampling rate then downsampled to 1000 Hz. Power spectral analyses were performed using the pwelch function in MATLAB using a 500 ms time window with a 50% overlap.

The relationship between spike firing times and LFP gamma phase was calculated by mean resultant length using the Circular Statistics Toolbox. Briefly, spikes were sorted and LFP traces were filtered using the continuous wavelet transform returning the instantaneous signal phase and amplitudes. Spike times were linearly interpolated to determine phase, with peaks and troughs of gamma defined as 0 and ±pi radians respectively. The resulting phase values were binned to generate firing probabilities, for each 20-degree interval. Cells were considered to be phase-locked if they had a distribution significantly different from uniform (p<0.05 circular Rayleigh test), with the strength of phase-locking calculated as the mean resultant length. All analyses were performed using MATLAB.

RNA Sequencing

Mice were killed and the brain tissue was freshly dissected out and the single cell suspension or nuclei was prepared. Single cell RNA libraries were prepared using the Chromium Next GEM Single Cell 3′ Kit v3.1 according to the manufacturer's protocol (10× Genomics). The generated scRNA-seq libraries were sequenced using NovaSeq. Gene counts were obtained by aligning reads to the mouse genome. All analyses were performed in R package following the methods as described previously (Mathys et al., 2019).

Statistical Analyses

Statistical analysis was conducted in Prism. Statistical significance was calculated as noted in the appropriate figure descriptions, using one-way ANOVA with a Two-stage linear step-up procedure of Benjamini, Krieger and Yekutieli post hoc analysis. Statistical significance was set at 0.05.

### Example 2

11-month-old APP.PS1 mice were either a) untreated as control, b) treated with GENUS alone (40 Hz light flicker delivered for 1 hour/day for 30 days), or c) a combination of GENUS (40 Hz delivered for 1 hour/day for 30 days) and levetiracetam (10 mg/kg body weight, intraperitoneal injection daily for 30 days), an agent shown to offer benefits in AD model mice. After the treatment mice were transcardially perfused with ice cold phosphate buffered saline (PBS) followed by 4% paraformaldehyde in PBS. Forty micron brain slices were prepared, and immunohistochemistry was then performed to evaluate the amyloid levels.

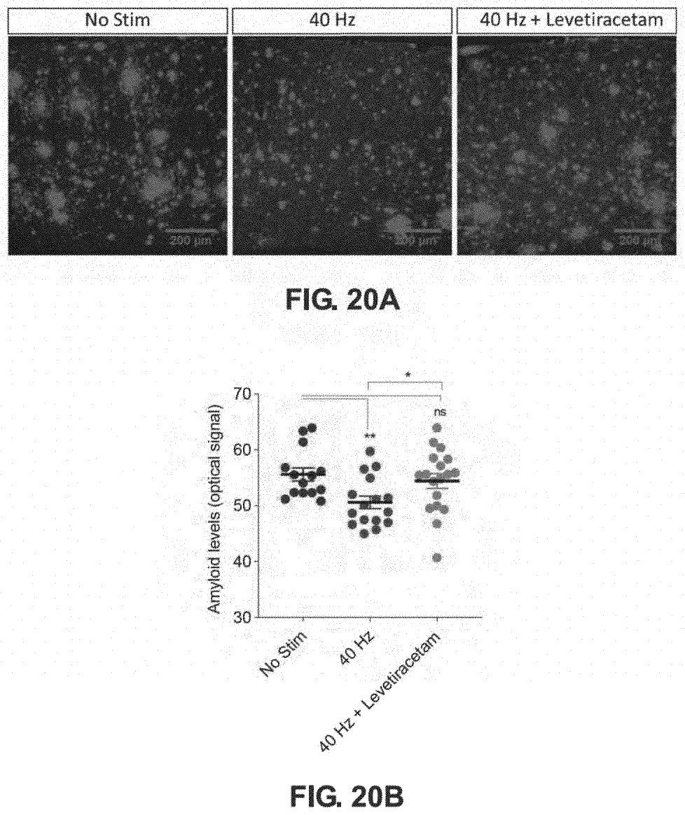

FIGS. 20A-20B illustrate the resulting amyloid levels in the cortex. GENUS reduced amyloid levels in the visual cortex compared to no stimulation control mice. With co-administration of levetiracetam with GENUS, on the other hand, it was observed that levetiracetam actually dampened or attenuated the effect of GENUS, with higher amyloid levels being observed in the visual cortex relative to treatment with GENUS alone.

Accordingly, it is not a given that coadministration of any drug/agent targeting a neurological condition with GENUS would result in an additive effect. Indeed, the inventive concepts disclosed herein relate to the inventors' findings that reducing pathology in the brain with a multi-modal approach that includes GENUS may be agent-dependent and/or disorder dependent.

### Example 3

Depleting CSF1R-sensitive microglia reduces inflammation and improves synaptic density in mouse models of Alzheimer's disease (AD). However, the effects of CSF1R-sensitive microglial depletion on synaptic and neural functions in AD remain largely unknown. Shown herein is that microglial depletion results in the decoupling of neuronal spiking from theta and gamma oscillations, which associates with changes in synaptic density but not amyloid levels in an amyloidosis mouse model. Furthermore, non-invasively driving gamma oscillations improves neural circuit function, and novel object recognition memory in CSF1R inhibited 5×FAD model mice. Molecular analysis revealed that entraining neural spiking and oscillations at gamma frequency in CSF1R inhibited 5×FAD mice improved intrinsic neural mechanisms by enhancing the expression of MEF2C, synaptic and extracellular matrix organizing genes, resulting in improved synaptic and extracellular architecture. This example highlights the indispensability of CSF1R-sensitive microglia in regulating the stochastic nature of neuronal activity and oscillations through a synaptic organization, and further that entraining spiking at gamma frequency in CSF1R inhibited AD mice is neuroprotective.

### INTRODUCTION

Microglia are the resident macrophages of the brain involved in sensing and regulating neuronal activity. Although the microglial function is necessary for normal brain functions, aberrant activation is thought to drive neuroinflammation and degeneration of synapses and neurons in Alzheimer's disease (AD). Specifically, microglia have been shown to be excessively proliferative and inflammatory in most parts of the brain, including the cortex and hippocampus, during AD disease progression. Further, microglia have been shown to facilitate the propagation of amyloid and tau during the early stages of disease progression. Therefore, studying the impact of altered microglial density and function is of general interest in the field. Accordingly, pharmacologically reducing microglia via inhibition of colony-stimulating factor 1 receptor (CSF1R), whose expression is crucial for microglial survival, attenuates neuroinflammation and neurodegeneration in mouse models of AD. However, despite numerous studies examining the effect of depletion of CSF1R-sensitive microglia on AD-associated pathological measures such as amyloid plaques, neuroinflammation, neurodegenerative phenotypes and gene expressions, little is known regarding how depletion affects neural functions in vivo. Recent studies that investigated the effect of systemic inhibition of CSF1R on neural oscillations showed a somewhat conflicting picture, with some studies reporting a lower threshold for seizure after CSF1R inhibitor treatments and others showing an anti-epileptic effect of CSF1R inhibitor in rodent models. Additionally, the CSF1R inhibitor Plx3397 (also known as pexidartinib) has been approved by the Food and Drug Administration (FDA) for the treatment of adult patients with symptomatic tenosynovial giant cell tumors—a rare disease characterized by joint/soft tissue neoplasms. Thus, understanding the impact of Plx3397 treatment on neural activity and function will help comprehend the relevance of CSF1R-sensitive microglia to neural activity and will also instruct future treatment strategies for neurodegeneration and/or tumors.

In this example, an amyloidosis mouse model, 5×FAD, is used and the effect of CSF1R-sensitive microglial removal by Plx3397 treatment is characterized. Plx3397 administration in 5×FAD mice aberrantly altered neural activity, manifesting as increased synaptic density and reduced percentage of neurons phase locked to gamma oscillations. These observations led to investigations into whether entraining neurons will improve the neural circuit alterations induced by Plx3397 administration. The gamma phase locking of neurons was increased by driving gamma using patterned sensory light stimulation. Repeatedly driving gamma in Plx3397 treated 5×FAD mice impacted the neuronal intrinsic gene expression profile to improve synaptic mechanisms, neural oscillations, and novel object recognition memory. These findings suggest that anti-inflammatory drugs, such as Plx3397 which show great promise for pathological modification, can be combined with non-invasive sensory stimulation to offer neuroprotection and improve memory functions in AD.

Results

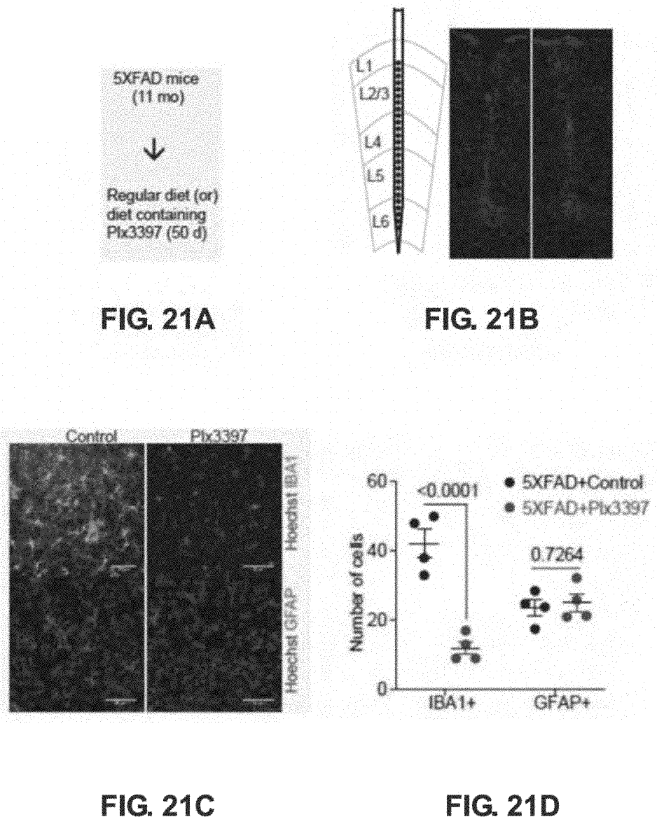

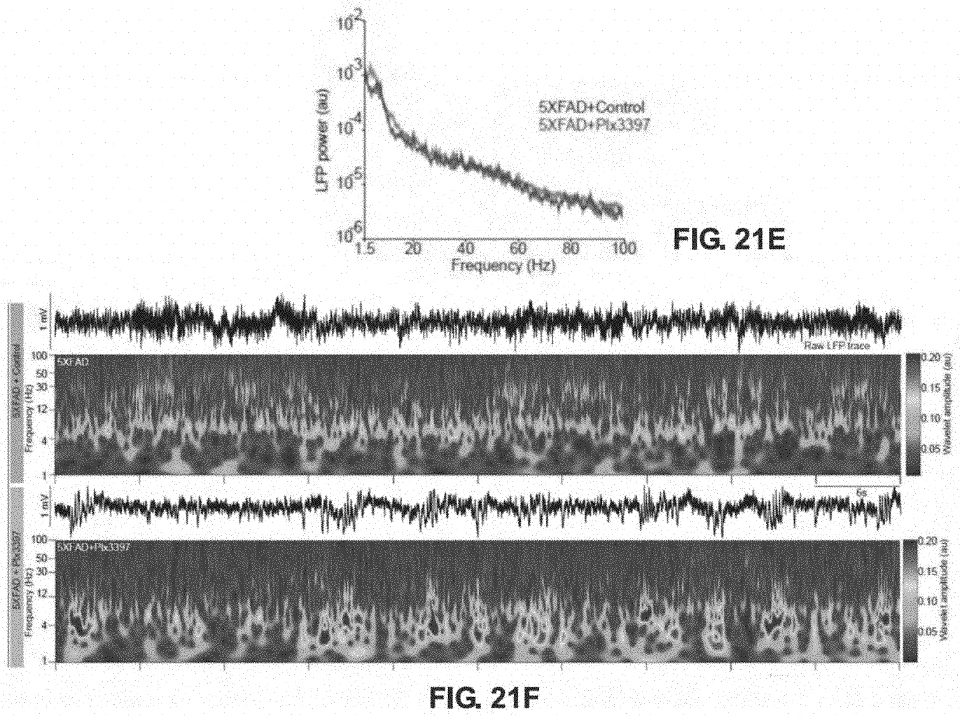

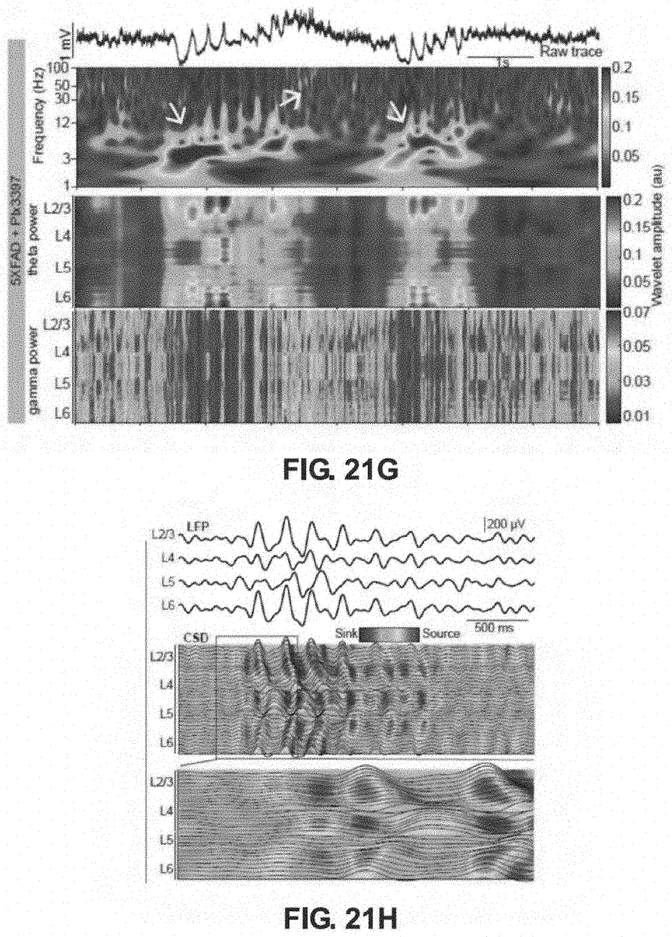

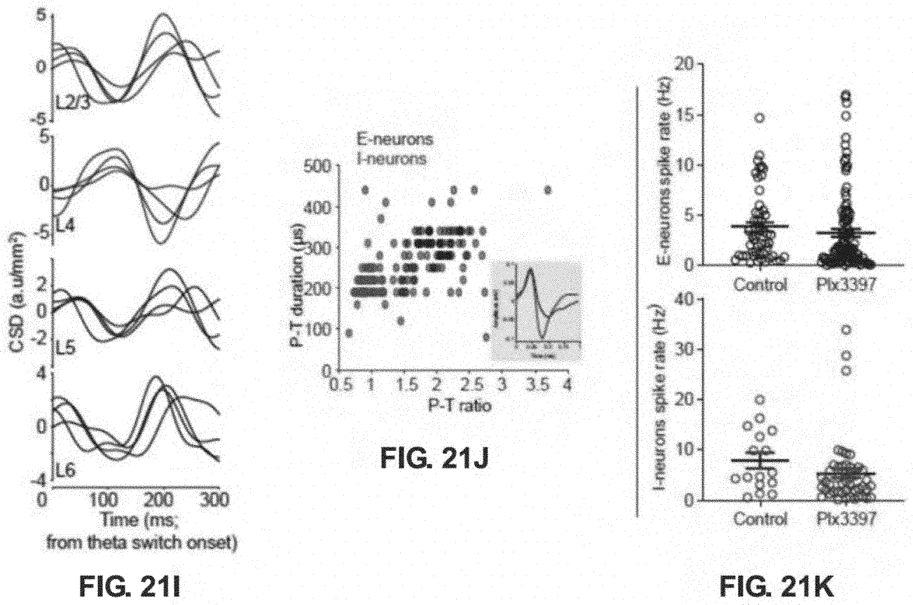

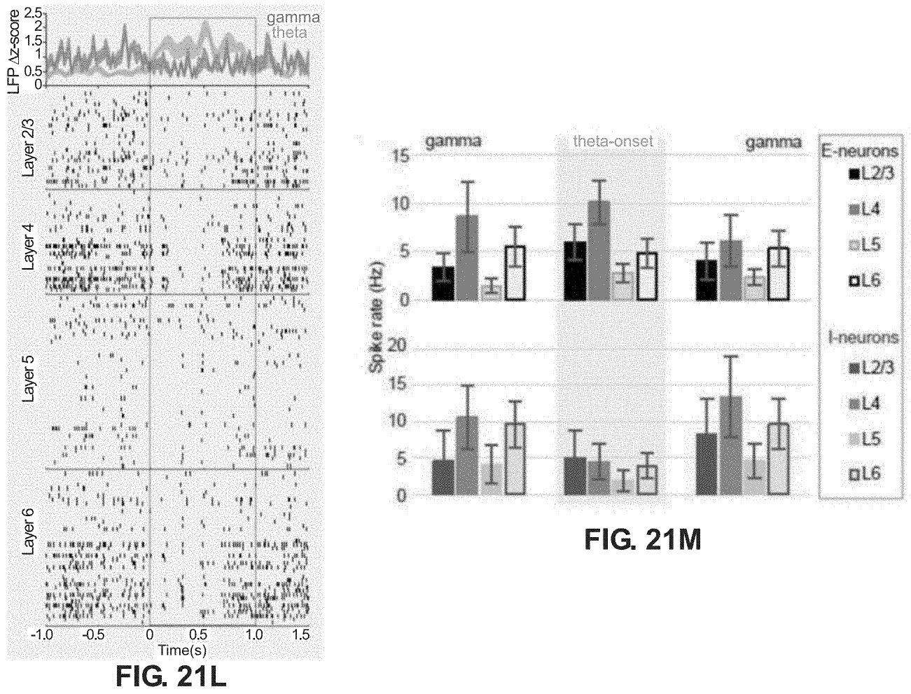

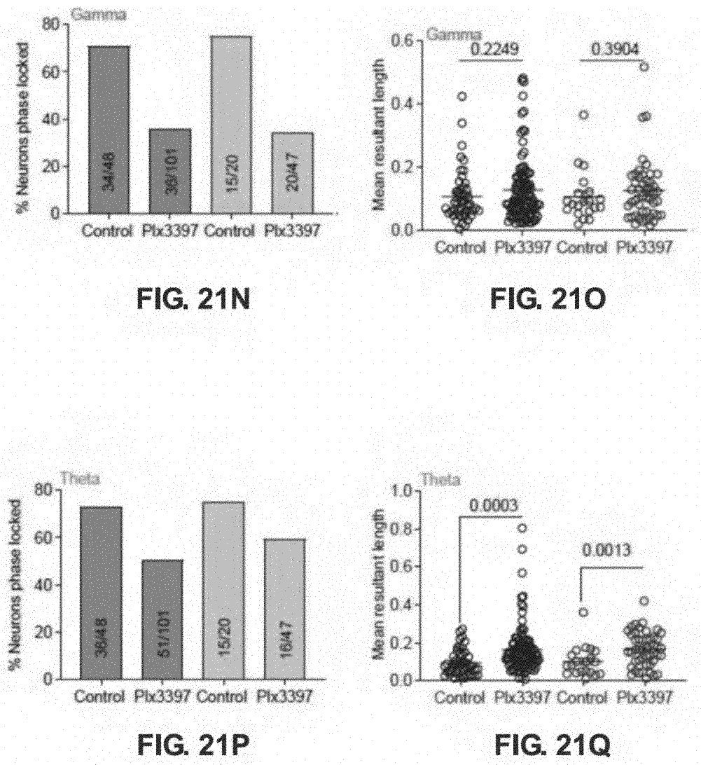

To study how CSF1R-sensitive microglia impact neural activity in the context of AD, an amyloidosis mouse model, the 5×FAD mice—which is a most commonly used mouse model for AD, was subject to a diet containing CSF1R inhibitor Plx3397 for 50 days (FIG. 21A). The CSF1R inhibitor Plx3397 (600 ppm) was employed, which was previously shown to effectively reduce microglia in vivo. Control mice were age-matched 5×FAD litters that received a regular diet. Electrophysiological recordings were performed using linear probes. Post-recording, the recording site in the visual cortex was verified by histology (FIG. 21), and microglia reduction was evaluated by assessing IBA1+ cell numbers. It was observed that Plx3397 treatment significantly reduced microglia (12±1.91 versus 42.25±4.09 IBA1+ cells in the control regular diet) (FIGS. 21C-21D). As controls, GFAP+ astrocytes (FIGS. 21C-21D) and CSF1R resistant progenitor-like MAC2+ microglia were examined and no difference between control and Plx3397 (FIGS. 26A-26B) was observed, suggesting Plx3397 reduced microglia without affecting astrocytes, and the remaining IBA1+ cells are CSF1R-resistant immature MAC2+ microglia.

To explore the effect of Plx3397 administration on neural oscillations, the power spectra of local field potential (LFP) was examined. While an overall spectral power change as a function of frequency in both groups was observed, there was no significant difference between control and Plx3397 groups (FIG. 21E). However, a modest non-significant trend in the LFP power with slight increase around the theta band (˜3-6 & ˜12 Hz) in the Plx3397 group was observed. Time-resolved analysis of the LFP revealed that chronic Plx3397 administration results in elevated power of gamma and theta that occur incongruently (FIGS. 21F-21G, FIG. 26C). In other words, Plx3397 treated 5×FAD mice exhibit an alternation of gamma (30-50 Hz) and theta (3-12 Hz) oscillations—hereinafter, these oscillations as referred to as gamma state and theta-bursts. These two oscillatory states were evident across all cortical layers (FIG. 21G). Conversely, control 5×FAD mice did not show such an alternating oscillatory state (FIG. 21F), consistent with previous reports. Further, the current source density analysis of laminar LFP revealed alternating sinks and sources (FIG. 21H). When aligned to the first rising phase of the LFP theta-burst, sinks were localized in layer 4, and the corresponding sources were observed in L2/3 and L5 (FIG. 21H). These observations led to study of the relationships between the aberrant LFP oscillations and neuronal spiking patterns after Plx3397 administration. To this end, single units were isolated and classified into either putative excitatory neurons (E-neurons) or interneurons (I-neurons) (FIG. 21J) using the parameters as described previously. 47 and 102 E-neurons and 20 and 47 I-neurons were isolated from the control and Plx3397 groups, respectively (FIG. 21K). While no differences were detected in the overall mean spiking rate of E-neurons and I-neurons (FIG. 21K), the spiking patterns in the Plx3397 group markedly differed between LFP theta-burst and gamma states (FIG. 21L). As shown in FIG. 21L single unit raster plots and the aggregated line plot (FIG. 26D), spiking rates of E-neurons and I-neurons during theta-bursts were significantly lower than that of the gamma state. As theta-bursts subside and gamma emerges, neuronal spiking increases substantially (FIG. 26D). At the population level, E-neurons maintained their spiking rates until the onset of the theta-burst; I-neurons reduced their overall spiking rate preceding and during the theta-burst (FIGS. 21L-21M). It was observed that layer 4 (L4) E-neurons transiently increased their spiking closer to the onset of the theta-burst (FIGS. 21L-21M), whereas I-neurons in layers 4 and 6 exhibited higher spiking during the gamma state (FIGS. 21L-21M). Overall, these data suggest that neurons alter their spiking patterns between LFP theta-bursts and gamma states rather than simply changing their overall mean spiking rate after CSF1R-sensitive microglial removal.

Further, the phase-locking of neurons was assessed to explore the relationship between neuronal spiking and LFP oscillations after Plx3397. The percentage of LFP phase-locked neurons and the strength of phase locking was quantified by Rayleigh statistics and mean resultant vector length, respectively. These analyses revealed that the percentage of both E-neurons and I-neurons in the Plx3397 group were less phase-locked to 30-50 Hz gamma oscillations than those neurons in the control 5×FAD group (FIG. 21N), without significant difference in the strength of those phase-locked neurons to gamma oscillations (FIG. 21O). Furthermore, it was observed that Plx3397 treatment significantly affected both the percentage of E-neurons and I-neurons phase-locked to 3-12 Hz theta oscillations (FIG. 21P) as well as the strength of theta phase-locking of neurons (FIG. 21Q) manifesting as a reduction in the % phase-locked neurons and increase in the phase-locking strength. Together these observations suggest that CSF1R-sensitive microglia play a crucial role in regulating the stochastic nature of the neuronal activity and their relationship to the LFP oscillations.

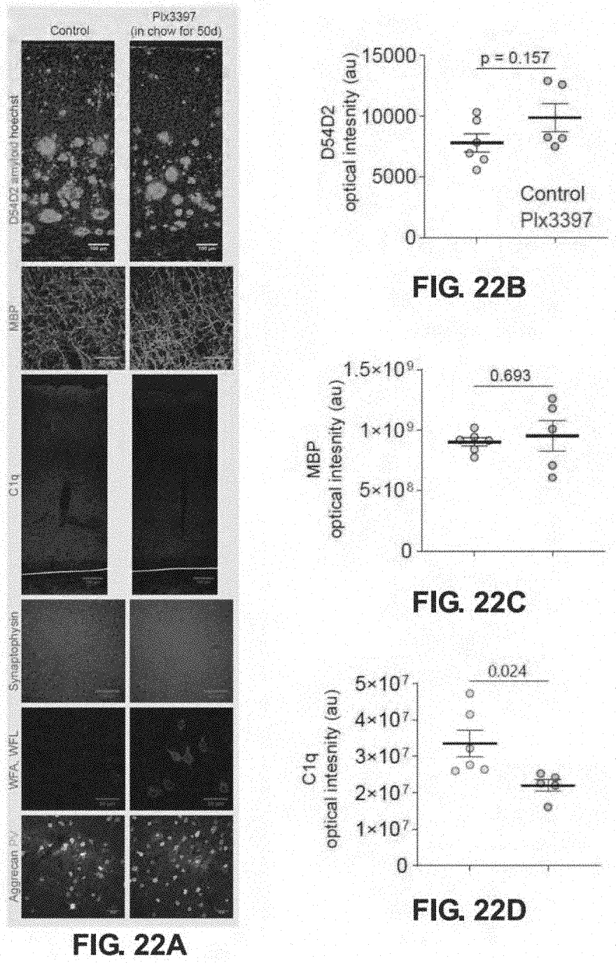

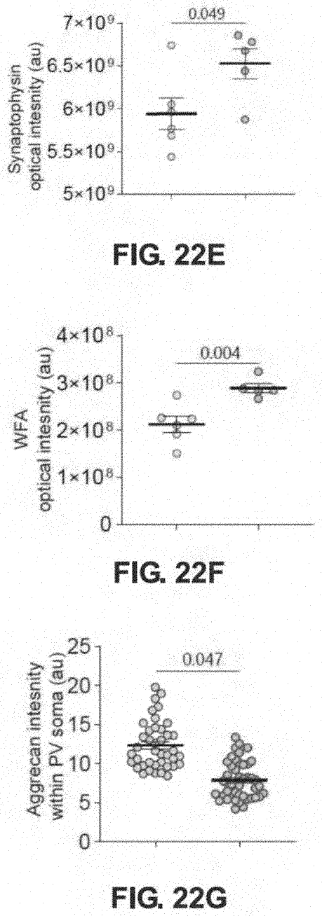

It was next sought to understand whether Plx3397 administration-dependent electrophysiological alterations were attributed to changes in amyloid plaque levels. Immunohistochemical (IHC) analysis revealed that chronic Plx3397 administration did not affect amyloid levels (arbitrary units (au), 9894±1181) as analyzed by the D54D2 positive amyloid signal in the visual cortex compared to control mice (7795±762.5) (FIGS. 22A-22B, FIGS. 27A-27B), consistent with previous findings in 5×FAD mice. In the cuprizone model of demyelination, Plx33977 administration preserved myelin. It was thus considered whether electrophysiological alterations are associated with myelin levels in Plx3397 treated 5×FAD mice. However, it was found that Plx3397 administration did not significantly affect the overall MBP signals (au, 953800000±1277515477 versus 904333333±34172764 in controls) (FIGS. 22A, 22C).

Turning next to neuronal and synaptic pathologies, IHC examination revealed that Plx3397 administration impacted synaptic integrity, manifesting as a reduction in C1q (au, 22040955±1586416 versus 33537105±3628262 in controls) (FIGS. 22A, 22D, FIGS. 27C-27D) and a concomitant increase in synaptophysin signals (au, 6524128709±177336289 versus 5937635931±183052579 in controls) (FIGS. 22A, 22E, FIGS. 27E-27F). These observations led to examination of the effect of Plx3397 administration on neuronal integrity. Extracellular matrix organization, specifically perineuronal nets (PNN), is thought to regulate the activity of neurons, and interestingly, microglia are shown to play crucial roles in this process. Indeed, it was observed that Plx3397 administration increased WFA+(Wisteria floribunda agglutinin, a PNN marker) PNN signals (au, 289400000±9384029 versus 213000000±16633300 in controls) (FIG. 22A, 22F), while aggrecan (a component in PNN) signals were reduced specifically around PV interneurons in Plx3397 administered 5×FAD mice (12.34±0.4791 versus 7.837±0.3549 in controls) (FIG. 22A, 22G). Collectively, these findings highlight the significant impact of CSF1R-sensitive microglial removal on neuronal architecture and further suggest the neuronal architectural alterations, but not amyloid level, after Plx3397 may contribute to the in vivo electrophysiological changes.

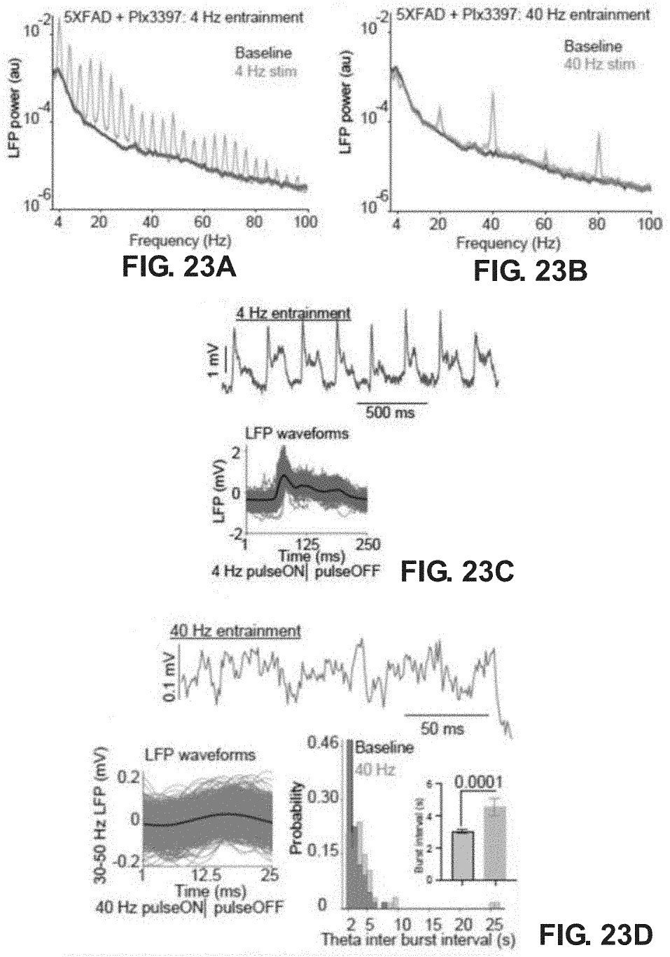

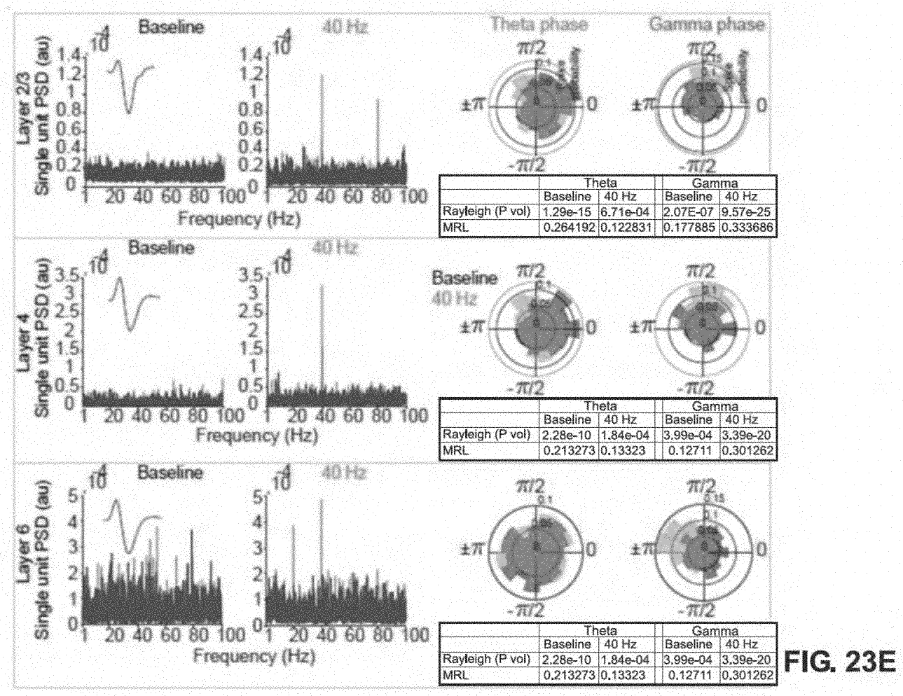

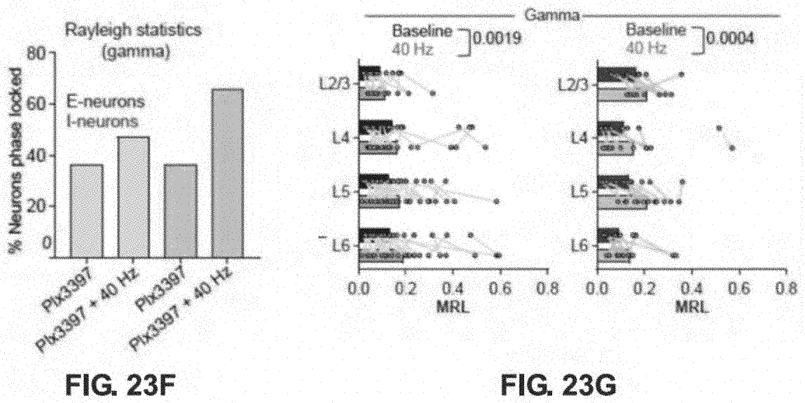

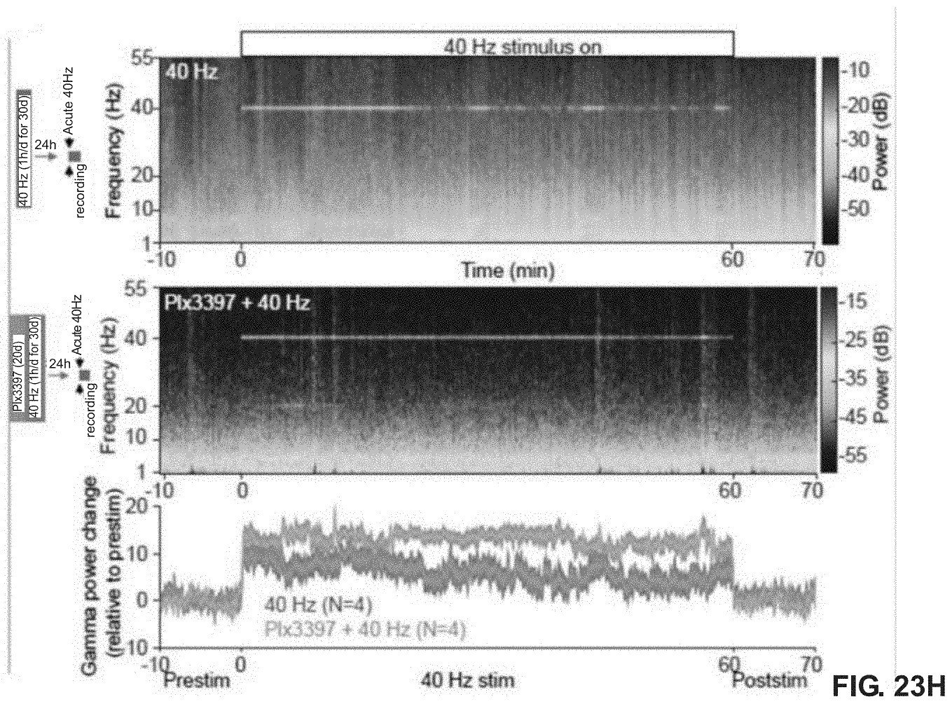

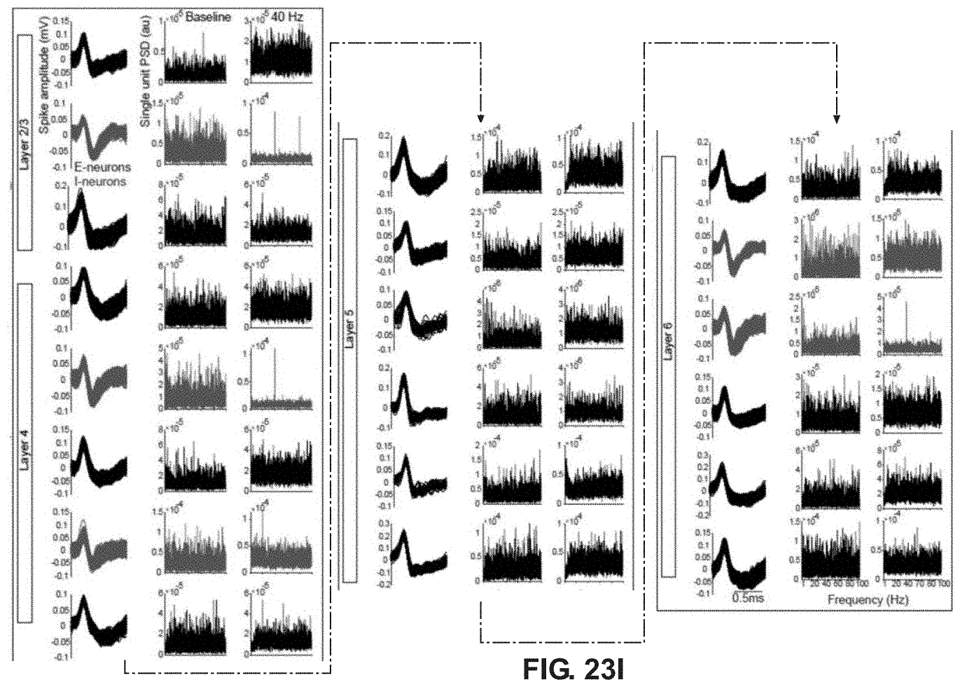

Given these observations, it was considered whether entraining neural spiking and oscillations at theta or gamma will morph the neural connectivity and oscillations in Plx3397 administered 5×FAD mice. Specifically, it was reasoned that evoking gamma oscillations, which are thought to be modulated by interneurons, could induce I-neuronal activity to improve the neuronal phase-locking and aberrant neural activity caused by the Plx3397 administration. It is also possible that evoked theta would impact neural phase-locking and oscillations in Plx3397 treated 5×FAD mice. To answer these questions, Plx3397 treated 5×FAD mice were exposed to either 4 Hz theta or 40 Hz gamma sensory stimulations and it was found that these sensory stimulations robustly induced LFP spectral power at the stimulated frequency (FIGS. 23A-23B). Thus, CSF1R-sensitive microglial removal did not abolish the ability of mice to entrain patterned theta or gamma frequency (i.e., 4 or 40 Hz) visual stimuli. Further, ascending and descending LFP phases were observed as a function of light pulse on and off periods during 4 Hz stimulation (FIGS. 23C-23D), consistent with previous findings. Although Plx3397 treated 5×FAD mice exhibited 4 Hz entrainment, assessment of the LFP waveform revealed an abnormal waveform with a sharp rise and a slow decay (FIG. 23C, FIGS. 28C-28D). On the other hand, it was observed that acute 40 Hz visual stimulation did induce physiological LFP waveforms and reduced the aberrant theta-bursts (FIG. 23D). Interestingly, Plx3397 administered 5×FAD mice exhibited higher power of 40 Hz entrainment compared to control 5×FAD mice (FIGS. 28D-28E) Together, these observations suggest that driving gamma could mitigate the aberrant neural oscillations after CSF1R-sensitive microglial removal.

To explore whether the reduction in aberrant theta-burst was due to rhythmic spiking of neurons during 40 Hz entrainment, neuronal spiking rhythmicity and phase locking to LFP oscillations during acute 40 Hz entrainment was characterized. Of the 47 I-neurons, 13 neurons showed 40 Hz entrainment as analyzed by 40 Hz peak in power spectral density of units (FIG. 23E). Although the mean spiking phase varied between units (FIG. 23E), spiking of 40 Hz rhythmic I-neurons occurred during the ascending phase of LFP gamma (FIG. 23E). Despite a subset of I-neurons entraining at 40 Hz (13 of 47), the phase-locking analysis showed that 40 Hz entrainment dramatically increased the percentage of I-neurons phase-locked to gamma (65.95% versus 34.04% in the baseline). Similarly, also observed was a marked increase in the percentage of gamma phase-locked E-neurons (46.53% versus 35.64% in the baseline) (FIG. 23F). Next, whether neurons in specific or all layers of cortex entrain 40 Hz and are phase-locked was examined. Overall, it was observed that I-neurons in L2/3, L4 and L6 show 40 Hz entrainment (FIG. 23E). Furthermore, both E-neurons and I-neurons distributed across all layers of the cortex (L2/3, L4, L5 and L6) showed enhanced gamma phase-locking strength (FIG. 23G). The theta phase-locked E-neurons, but not I-neurons, were modestly reduced during 40 Hz entrainment, without a significant difference in the strength of the theta phase-locking of neurons (FIGS. 28F-28G). Together, these results suggest that acute 40 Hz sensory stimulation (termed Gamma Entrainment Using Sensory stimulation; GENUS) induces 40 Hz rhythmic spiking of I-neurons in many cortical layers and that rhythmic modulation of I-neurons is sufficient to enhance the percentage of neurons phase locked to gamma oscillations and further reduce the aberrant alterations of oscillations caused by Plx3397 treatment.

Although 40 Hz entrainment was observed during acute sensory stimulation, it was desired to verify that chronic GENUS is also possible in control and Plx3397 treated 5×FAD mice. To this end, control diet- and Plx3397-treated 5×FAD mice were subject to 40 Hz stimulation one hour per day for 30 days and performed electrophysiological recordings. It was observed that 40 Hz stimulation robustly induced 40 Hz entrainment across the entire one-hour stimulation period in these mice (FIG. 23H), and I-neurons in L2/3, L4 & L6 in the Plx3397+GENUS group were 40 Hz rhythmic (FIG. 23I). Interestingly, mice that were treated with the combination of Plx3397 diet and GENUS exhibited a stronger 40 Hz entrainment than mice that received only GENUS (FIG. 23H).

How does regulating gamma oscillations affect synaptic and neural circuit function in Plx3397 treated 5×FAD mice? Specifically, it was asked what gene expression patterns are after the Plx3397 treatment and how gamma entrainment impacts such a signature. To address these questions, an unbiased RNA sequencing approach was utilized. 5×FAD mice were treated, via oral delivery in mouse chow, with the Plx3397. Following 20 days of administration of Plx3397 chow, untreated- and Plx3397-treated 5×FAD mice (which continued Plx3397 administration) were subjected to 30 days of daily GENUS (FIG. 24A). Following completion of the treatments, microglial density was quantified between 1) untreated (control), 2) Plx3397, 3) GENUS and 4) Plx3397+GENUS treated 5×FAD mice (FIGS. 24A-24D). A significant reduction in IBA1+ microglia number and % area covered by IBA1 in Plx3397 (microglia number & %, 47.18±5.44 & 7.27±0.45), and Plx3397+GENUS (24.34±2.41 & 4.59±0.78) groups compared to controls (100±5.12 & 13.72±0.89) (FIGS. 24B-24D) was found. Also observed was a reduction in IBA1 signal in the GENUS group (84.0±6.42 & 9.17±0.85) (FIGS. 24C-24D), which is consistent with previous observation in CK-p25 mice. After the treatments, the visual cortex was dissected, and single nucleus RNA-sequencing was performed.

All major brain cell types were identified based on marker genes and overall gene expression patterns (FIGS. 24E-24F). The overlap and biological functions of differentially expressed genes (DEG) in E-neuronal clusters was examined, which also showed the highest number of DEGs (FIGS. 24G-24K). The genes down and upregulated in the combined administration of the Plx3397 and GENUS group were also compared to that of the Plx3397 group alone (FIGS. 24G-24K). Consistent with electrophysiological and IHC analyses, abnormal synaptic transmission genes were upregulated after Plx3397 administration alone; daily GENUS in Plx3397 administered 5×FAD downregulated these genes in E-neurons (FIG. 24G). Genes related to protein phosphorylation (e.g., Rock1, Rock2, Grk3, Mark2) were downregulated in E-neurons and I-neurons after Plx3397+GENUS (FIG. 24H). These kinases are implicated in neurodegenerative diseases and their inhibition has been shown to offer protective effects. Furthermore, these observations are also consistent with previously reported findings wherein chronic GENUS reduced overall protein phosphorylation levels in CK-25 and P301S tau mouse models of neurodegeneration.

In addition, GENUS rescued the expression of head and brain development genes in Plx3397 treated 5×FAD mice (FIG. 24I). Plx3397+GENUS compared to Plx3397 administration increased genes related to synaptic plasticity (e.g., Cf11, Cp1x2, Snap25, Unc13a), learning and memory (Mef2c, Map1a, Pten, Snap25, Ube3a, Slc24a2, Pak5), general synaptic organization and function (e.g., Cacnala, Cf11, Col4a1, Sparcll, Epha4, Gabral, Myo6, Pten, Sptbn2, Pclo, Chd4, Gpm6a, Lrfn5, Erc2, Unc13a, Cdh11, Dst, Plec, Thyl, Mef2c) in E-neurons (FIG. 24J). Furthermore, Mef2c, a transcription factor, was one of the highest upregulated genes after combined administration of Plx3397 and GENUS in both E- & I-neurons (FIG. 24K). Recent findings demonstrated that Mef2c regulates synaptic genes, intrinsic neuronal functions and confers resilience to neurodegeneration. To validate the transcriptomic findings, immunohistochemical (IHC) staining of MEF2C was performed, and it was found that Plx3397+GENUS treatment significantly increased the expression of MEF2C compared to control, Plx3397, and GENUS alone groups (FIGS. 24L-24M). Together, these findings are consistent with a view that repeated GENUS in Plx3397 administered 5×FAD mice improved the gene expressions impacting neural function. In addition to intrinsic neuronal mechanisms, glia morph the neuronal circuit architecture by various mechanisms. So, to gain insight into how GENUS+Plx3397 administration affects the glial cells to modify neural functions, the DEGs in glial clusters were examined by performing a complementary single-cell RNA-seq which is shown to capture more glial cells. Microglia and oligodendrocytes showed higher DEGs in scRNA-seq (FIGS. 29A-29H). It was observed that Plx3397+GENUS administration impacted the expression of genes related to extracellular matrix organization in addition to myelination-related genes in both microglia and oligodendrocytes compared to Plx3397 administration alone in 5×FAD mice (FIGS. 29E-29H).

These observations point to synaptic connectivity, PNN extracellular architecture, and myelination as biological processes that GENUS impacts to improve the outcomes in Plx3397 administered 5×FAD mice. To further validate this, additional biochemical analyses were performed. Western blot analysis of synaptic proteins revealed that Plx3397+GENUS administration improved the overall levels of synaptic proteins such as synaptophysin (100±12.03, 185.3±23.83, 142.1±9.22, &190.5±27.31 in control, Plx3397, GENUS, Plx3397+Genus groups, respectively) and vGLUT1 (100±7.30, 130.1±10.05, 108.7±6.584 & 137.8±9.176) (FIGS. 25A-25C). IHC analysis showed Plx3397 (109.9±2.235), GENUS (108.6±3.01), and Plx3397+GENUS (122.6±4.14) administration increased vGAT synaptic puncta compared to control (100±2.71) (FIGS. 25D-25E). While vGAT puncta did not differ between Plx3397 and GENUS groups, a stronger increase in vGAT puncta in 5×FAD mice that received combination Plx3397+GENUS in the visual cortex (FIG. 25E) was observed, suggesting higher levels of inhibitory synaptic connectivity after GENUS in Plx3397 treated 5×FAD mice. Next, it was observed that Plx3397 (130.4±7.178), GENUS (116.2±5.225) and Plx3397+GENUS (119.0±6.325) all increased the WFA signals with the highest levels in Plx3397 alone group compared to controls (100±4.655) (FIGS. 25D, 25F). Further, WFA content was higher within the microglia in the Plx3397 group but not in Plx3397+GENUS compared to control mice (FIGS. 30A-30B), suggesting an active role of microglia in organizing PNN in L4, and further that GENUS transforms this microglial phenotype. Synaptic input arriving within the PNN of parvalbumin (PV) interneurons and the PNN architecture are shown to modulate the activity of PV interneurons robustly. Triple labeling (PV, WFA and VGLUT1) was performed and the excitatory presynaptic marker vGLUT1 in the WFA of PV interneurons was examined; it was observed that Plx3397+GENUS (366.6±12.78), & GENUS (363.0±16.64), but not Plx3397 alone (332.7±13.14 versus 313.4±13.20 in controls), significantly increased vGLUT1 synaptic puncta (FIGS. 25D, 25G). Next, although the total MBP levels was not affected (100±19.58, 132.4±30.10, 140.2±25.31, & 133.4±22.13 in control, Plx3397, GENUS, Plx3397+Genus groups, respectively), GENUS increased myelin ensheathment of axons of PV interneurons in Plx3397 administered 5×AFAD mice (20.75±1.45, 25.44±2.83, 37.91±4.56, & 31.3±3.21) (FIGS. 25D, 25H, 25I and FIG. 30C). Collectively, these findings show improved synaptic connectivity and axonal myelination of PV interneurons after repeated GENUS in Plx3397 administered 5×FAD mice. Given these observations, whether these treatments had any effect on neuronal density was examined, and it was found that 5×FAD mice with Plx3397+GENUS had higher NeuN+ neuronal density (FIGS. 25D, 25J), suggesting that CSF1R inhibition together with GENUS can provide neuroprotective effects in 5×FAD mice.

Finally, a behavioral analysis was performed to assess whether the increased genes related to learning and memory observed in both excitatory and interneuron clusters after Plx3397+GENUS were associated with improved learning and memory (FIG. 24J). Mice were tested in an open field (OF), followed by a novel object recognition (NOR) test of memory after Plx3397, GENUS, and Plx3397+GENUS treatments. No changes in the time spent in the center of the OF arena or locomotor activity was observed (FIGS. 25K, 25L and FIG. 30D). In the NOR test, GENUS (60.30±3.58) and Plx3397+GENUS (72.69±3.11) treated 5×FAD mice showed an improved preference for the novel object compared to chance level (50%), while this was not observed in control (51.33±4.15) and Plx3397 alone group showed a trend (57.55±7.00) (FIGS. 25K, 25M).

The finding of improved NOR memory after Plx3397+GENUS administration was replicated using the CK-p25 mouse model of neurodegeneration. Specifically, CK-p25 mice were chronically treated with Plx3397 and GENUS, and microglial depletion and behavioral performance were assessed. Plx3397 (44.19±2.86), GENUS (81.69±6.29), and Plx3397+GENUS (35.54 2.04) administrations reduced IBA1+ microglial cells compared to control CK-p25 mice (100 21.17) (FIGS. 30E-30G), consistent with the observations in 5×FAD mice. It was observed that, compared to control CK-p25 mice (54.82±3.70), Plx3397 (70.52±2.89), GENUS (69.90±5.88), and Plx3397+GENUS (69.63±2.64) significantly improved novel object recognition memory in the NOR test without affecting open field exploration or anxiety levels (FIGS. 30E, 30H, 30I). Overall, these data suggestthatPlx3397+GENUS can improve novel object recognition memory in two different mouse models of neurodegeneration.

### DISCUSSION

Understanding of the importance of microglia on neural circuit function and oscillations is evolving. Oscillations emerge when groups of cells synchronize their transmembrane currents and neuronal spiking. The spiking of many single neurons is synchronized such that they spike at a preferred phase of the oscillations. In particular, theta and gamma oscillations in the visual cortex are well accepted to play roles in attention, learning, and memory. Described herein is a previously uncharacterized function of microglia on neural oscillations: 1) in the absence of CSF1R-sensitive microglia neuronal spiking and theta-gamma oscillations are decoupled, 2) this decoupling is closely associated with changes in genes related to synapse organization, and 3) driving gamma oscillations and gamma rhythmicity of neurons improves neural functions and transforms the gene expression signatures leading to neuroprotective and improved learning and memory effects in Plx3397 treated 5×FAD mice.

L4 neurons in the primary visual cortex (V1) receive robust input from the lateral geniculate nucleus (LGN). Cortical layer-specific neuronal spiking pattern with L4 interneurons was observed showing dramatic reductions while E-neurons increased spiking rate during the onset of aberrant theta-burst in Plx3397 administered 5×FAD mice, indicating abnormal synaptic connectivity and communication between V1-LGN in CSF1R-sensitive microglia removed 5×FAD mice. Thus, microglia play an indispensable role in synaptic and circuit organization in adult animals, consistent with their role in orchestrating V1-LGN connectivity during development. Although more synaptic markers are evident after CSF1R-sensitive microglial removal, L4 PV interneurons are aberrantly altered in their synaptic input architecture, such as changes in PNN. Shown herein is that patterned sensory stimuli that evoke gamma in the visual cortex significantly morph the synaptic connectivity within PNN of L4 PV interneurons, which is closely associated with improved neural oscillations in Plx3397 treated 5×FAD mice. Further, enhanced synaptic density after microglial removal is thought to be attributed to reduced synaptic pruning by microglia, and this aberrantly regulates neural communications. Interestingly, the unbiased gene expression analysis suggests that driving gamma induces intrinsic neuronal mechanisms to enhance the expression of synapse-related genes. Thus, neuronal, in combination with glia-dependent improvement in synaptic connectivity, offer neural circuit protection over strictly microglial-dependent increases in synaptic density by CSF1R inhibition.

Consistent with previous findings, it was observed that GENUS reduced microglial density in both 5×FAD and CK-p25 models of neurodegeneration, but it should be noted that the reduction is not as dramatic as Plx3397 administration. Previously, GENUS was thought to act to transform microglia to provide beneficial effects; however, recent observations indicate that chronic GENUS reduces microglial density and inflammatory response. Thus, these findings would be consistent with the view that lowering microglia would offer benefits in AD.

These findings suggest that anti-inflammatory drugs, such as Plx3397 which show great promise for pathological modification, can be combined with non-invasive sensory stimulation to offer neuroprotection and cognition in AD. Therefore, these findings provide proof of principle that a combination of microglial pharmacology and brain stimulation is a promising strategy to improve AD and, possibly also, tumor outcomes.

Animal Models

All the experiments were approved by the Committee for Animal Care of the Division of Comparative Medicine at the Massachusetts Institute of Technology (MIT) and carried out at MIT. Tg(Camk2a-tTA), and Tg(APPSwFlLon, PSEN1*M146L*L286V) were obtained from the Jackson laboratory. Tg(tetO-CDK5R1/GFP) was generated. 5×FAD mice were 10-12 months old and CK-p25 mice were 8 months-old prior to commencement of experiments. Equal numbers of male and female CK-p25 in each group was used. female 5×FAD mice were used for RNA-sequencing experiment, and male 5×FAD mice for all other experiments.

Experimental Treatment

CSF1R inhibitor Plx3397: Plx3397 (Pexidartinib; CAS #: 1029044-16-3, medkoo.com/products/4501) drug was obtained from Medkoo Biosciences (Morrisville, N.C., USA). Plx3397 was then irradiated and premixed into rodent diet at 600 ppm (PMI RMH 3000 5P76 rodent diet with 0.06% Plx3397). A red food color is added to the Plx3397 diet. These later processes were completed by Envigo Teklad Diets (Madison, Wis., USA). Plx3397 diet was stored in a cold room until use.

Plx3397 administration: Mice were introduced into clean new cages, and regular diet were replaced with diet containing Plx3397. Only experimenters A.C, M.S, and C.P (but no animal care takers) handled or changed cages during the entire experimental procedures. Cages were changed once weekly. Mice were given Plx3397 diet and water ad libitum, just as the regular diet control mice throughout the experiment.

GENUS stimulation: Light flicker stimulation was delivered as previously described. Mice were transported from the holding room to the flicker room, located on adjacent floors of the same building. Mice were habituated under dim light for 20 min before the start of the experiment, and then introduced to the stimulation cage (similar to the home cage, except without bedding and three of its sides covered with black sheeting). All GENUS protocols were administered on a daily basis for 1 h/d for the number of days as specified. Mice were allowed to freely move inside the cage but did not have access to food or water during the 1 hour light flicker. An array of light-emitting diodes (LEDs) was present on the open side of the cage and was driven to flicker at a frequency of 40 Hz with a square wave current pattern using an Arduino system. The luminescence intensity of light that covered inside the total area of GENUS stimulation cage varied from −200-1000 lux as measured from the back and front of the cage (mice were free to move in the cage). After 1 h of light flicker exposure, mice were returned to their home cage and allowed to rest for a further 30 min before being transported back to the holding room. No-stimulation control mice underwent the same transport and were exposed to similar cages with similar food and water restriction in the same room, but experienced normal room light for 1 hour. Experimenters who stimulated the mice were male.

Experimental Groups Description:

Control 5×FAD mice: Mice received regular rodent diet and water ad libitum. Mice also received control sensory stimulation as described above.

Plx3397 5×FAD mice: Mice received Plx3397 and water ad libitum for 50 days.

GENUS 5×FAD mice: Mice were subjected to 30 days of daily GENUS (1 h/d).

Plx3397+GENUS 5×FAD mice: Following 20 days administration of Plx3397 chow, 5×FAD mice were subjected to 30 days of daily GENUS (1 h/d). Mice were still maintained on Plx3397 diet during the 30 days of GENUS stimulation.

Control CK-p25 mice: p25 was induced by replacing the doxycycline diet with a regular rodent diet. Mice also received control sensory stimulation as described above. This treatment procedure (regular diet+control stimulation) was administered for 6 weeks.

Plx3397+GENUS CK-p25 mice: p25 and induced while also inhibiting CSF1R by replacing the doxycycline diet to Plx3397 rodent diet. In addition, CK-p25 mice were also subjected to daily GENUS (1 h/d) for 6 weeks simultaneously.

Open Field (OF) and Novel Object Recognition (NOR) Test

For OF, mice were introduced into an open field box (dimensions: length=460 mm, width=460 mm and height=400 mm; TSE-Systems) and were tracked using Noldus (Ethovision) for 12 min, with time spent in the center and peripheral area of the arena measured. NOR occurred on the following day, when mice were re-introduced into the same open field box which now additionally contained two identical novel objects and were allowed to explore the objects for 7 min (novel object habituation). Mice were then placed back in their home cages for 20 min after the last exploration. They were then returned to the same arena, with one of the two objects replaced with a new object. Mouse behavior was monitored for 7 min. Time spent exploring both the familiar and novel objects was recorded using Noldus and computed offline. Percentage of novelty preference index was calculated as follows: time exploring novel object (Nt) divided by total time exploring novel and familiar (Ft) objects and presented in %−{[Nt/Nt+Ft]*100}.

Immunohistochemistry

Mice were transcardially perfused with 40 mL of ice-cold phosphate-buffered saline (PBS) followed by 40 mL of 4% paraformaldehyde (PFA; Electron Microscopy Sciences, Cat #15714-S) in PBS. Brains were removed and post-fixed in 4% PFA overnight at 4° C. and transferred to PBS prior to sectioning. Brains were mounted on a vibratome stage (Leica VT 1000S) using superglue and sliced into 40 mm sections. Slices were subsequently washed with PBS and blocked using 5% normal donkey serum prepared in PBS containing 0.3% Triton X-100 (PBST) for 2 hours at room temperature. Blocking buffer was aspirated out and the slices were incubated with the appropriate primary antibody (prepared in fresh blocking buffer) overnight at 4° C. on a shaker. Slices then were washed three times (10 min each) with the blocking buffer and then incubated with the Alexa Fluor 488, 555, 594 or 647 conjugated secondary antibodies for 2 hours at room temperature. Following three washes (15 min each) with blocking buffer and one final wash with PBS (10 min), slices were mounted with fluromount-G (Electron microscopic Sciences).

Antibodies: IBA1 (Synaptic Systems, Cat #234 004, dilution-1:500; Wako Chemicals, Cat #019-19741, dilution-1:500), GFAP (Thermo Fisher Scientific, Cat #130300, dilution-1:500), MEF2C (Cell Signaling Technology, Cat #5030T), MAC2 (Cedarlane Labs, Cat #CL8942AP, dilution-1:500), vGAT (Synaptic Systems, Cat #131 013, dilution-1:500), vGLUT (Synaptic Systems, Cat #1135 302, dilution-1:500), NeuN (Synaptic Systems, Cat #266 004, dilution-1:1000), MHC2 (EMD Millipore, Cat #MABF33, dilution-1:500), C1q (Abcam, Cat #ab182451, dilution-1:500), synaptophysin (Sigma, Cat #S5768). The following combination of secondary antibodies were used: (1) Alexa Fluor 488, 594 and 647, (2) Alexa Fluor 555 and 647, (3) Alexa Fluor 594 and 647, or (4) Alexa Fluor 488 and 647. All secondary antibodies were obtained from Invitrogen. Biotinylated Wisteria Floribunda Lectin (Vector Laboratories, Cat #B-1355, dilution-1:500) followed by streptavidin conjugated Alexa Fluor 594 (Thermo Fisher Scientific, Cat #S32356, dilution-1:1000) was used to examine WFA.

Images were acquired using either LSM 710 or LSM 880 confocal microscopes (Zeiss) with 10×, 20×, or 40× objectives at identical settings for all conditions. Images were quantified using Imarisx64 9.3 or Imarisx64 9.7 (Bitplane, Switzerland). For each experimental condition, two coronal sections per mouse from the indicated number of animals were used. The averaged values from the two to four images per mouse were used for quantification. The experimenter blinded to the treatment conditions performed all the image processing and quantification.

C1q and MHC2 signal intensity: Using an LSM 710 with a 20× or 40× objectives, z stacks of the entire slice thickness 40 mm (40 images from each field) were acquired. The signal intensity was measured in Imaris.

Microglia: Iba1 immunoreactive cells were considered microglia. Using an LSM 710 or LSM 880 with a 10× (for Iba1+ cell counts) or 40× (for morphological analysis) objective z stacks of the entire slice thickness 40 m with 0.5 m step size were acquired. Imaris was used for 3D rendering of images to quantify the total volume of microglia. MAC2+ cells were counted manually using Image J.

MEF2C: LSM 710, with a 40× objective, was used to acquire the images. The entire m thickness of the slices was acquired in Z stacks 40 per image. MEF2C optical signal was measured using Image J.

NeuN positive cell: All images were acquired in Z stacks—10 per image (step of 2 μm) and were quantified. The spot-count inbuilt function in multi-point tool in Imarisx64 9.3 was used to count cells automatically.

vGAT and vGLUT1 puncta: LSM 710, with a 40× objective, was used to acquire the images. The entire 40 m thickness of the slices was acquired in Z stacks—80 per image (step of 0.5 μm). The spot-count inbuilt function in Imarisx64 9.3 (cohortl) and 9.7 (cohort 2) was used to count cells automatically.

Western Blotting

The brain was perfused with PBS and fixed with 4% PFA. Visual cortex was dissected out into 1.5 ml Eppendorf tube containing 100 μl of TS buffer (600 mM Tris-HCl, pH 8, and 2% SDS). The tissue was homogenized thoroughly using a handheld gun. The homogenate was incubated at 90 degree C. for 2 hours (at 500 rpm in TS buffer). The homogenate was then centrifuged at 1000 g for 1 min at room temperature, and the upper 60 ul of sample was transferred to a new Eppendorf tube. Laemmli sample buffer (Bio-Rad, Cat #1610747) was added to the sample. Samples were loaded onto 4-20% polyacrylamide gels (Bio-Rad, Cat #4561096 or 4561094) and electrophoresed (Bio-Rad). Protein was transferred from acrylamide gels to nitrocellulose membranes for 12 min (Semi-dry system, Bio-Rad). Membranes were blocked using BSA (5% w/v) diluted in TBS containing 0.1% Tween-20 (Sigma-Aldrich, Cat #P9416) (TBSTw), then incubated in primary antibodies overnight at 4° C. The following day, they were washed three times with TBSTw and incubated with horseradish peroxidase-linked secondary antibodies (Jackson Immuno Research, Cat #211-032-171, dilution-1:5000) at room temperature for 2 hours. After three further washes with TBSTw, membranes were treated with chemiluminescence substrates Western-Bright Quantum kit (Advansta, Cat #K-12042-D20) and the blots were visualized (Chem doc, Bio-Rad). Signal intensities were quantified using ImageJ 1.46q and normalized to values of loading control.

In Vivo Electrophysiology

Mice were anaesthetized with isoflurane, restrained in a stereotactic apparatus and craniotomies were made exposing the visual cortex. Specifically, a 2×2 mm piece of skull was removed using a dental drill, which was above the V1 (stereotaxic 826 coordinates relative to bregma; AP −3.2; ML+2.5); during this entire procedure, the dura was kept intact and moist with saline. Following the skull removal from above both the right V1, two additional drilling holes above the frontal cortex were made and two skull screws were placed. Recording probes (Neuronexus, Cat #A1×32-5 mm-25-177-CM32, A1×16-3 mm-50-177-CM16LP) were then fitted to the stereotactic apparatus and aligned to the craniotomy and slowly lowered to ˜50 m above the cortical target depth. The probe was grounded to skull screw above the cerebellum. Petroleum jelly (Vaseline, 100% white petrolatum) was gently applied on the cranial window without touching the probe/electrodes, which protected both the brain and probe/electrode. Next, the probe was further lowered and/or adjusted to reach the target depth. Finally, the probe was cemented on the skull with dental cements, first with a metabond (Parkell, C&B Metabond Quick Adhesive Cement System, #836 SKU:S380) followed by a dental cement from Steolting (#51459). Mice were allowed to recover for a period of 4 days.

Following a 2-3-day habitation period for the recording, recordings commenced with the animal allowed to move freely in their home cages. Data were acquired using Neuralynx SX system (Neuralynx, Bozeman, Mont., USA) and signals were sampled at 32,000 Hz. The position of animals was tracked using red light-emitting diodes affixed to the probes. At the conclusion of the experiment, mice underwent terminal anesthesia and electrode positions were marked by electrolytic lesioning of brain tissue with 50 mA current for 10 s through each electrode individually, to confirm their anatomical location.

Spikes: Single units were manually isolated by drawing cluster boundaries around the 3D projection of the recorded spikes, presented in SpikeSort3D software (Neuralynx). Cells were considered pyramidal neurons if the mean spike peak-t0-trough length exceeded 220 ms and had a higher peak-to-trough ratio.

Data analyses: LFPs were first filtered to the Nyquist frequency of the target sampling rate then down-sampled to 1000 Hz. Power spectral analyses were performed using the pwelch function in MATLAB using a 500 ms time window with a 50% overlap.

Time-frequency representation of LFP: The LFP data were down sampled to 1,000 Hz. For the calculation of the wavelet power spectrum, the continuous wavelet transforms (CWT) was applied to the LFP using complex Morlet wavelets returning amplitudes at 226 intervals between 1-100 Hz. CWT based wavelet power spectrum was shown in FIG. 21A, FIG. 26C, and FIG. 27C. For visualizing 40 Hz entrainment at finer frequency resolution, multitaper spectral analysis using Chronux toolbox was used.

Single unit—LFP phase locking: The relationship between spike spiking times and LFP gamma phase was calculated by mean resultant length using the Circular Statistics Toolbox MATLAB File Exchange Function.

Briefly, spikes were sorted and LFP traces were filtered using the continuous wavelet transform returning the instantaneous signal phase and amplitudes. Spike times were linearly interpolated to determine phase, with peaks and troughs of gamma defined as 0 and ±pi radians respectively. The resulting phase values were binned to generate spiking probabilities, for each 20-degree interval. Cells were considered to be phase-locked if they had a distribution significantly different from uniform (p<0.05 circular Rayleigh test), with the strength of phase-locking calculated as the mean resultant length. All analyses were performed using MATLAB. All in vivo electrophysiological analyses were conducted in MATLAB (Mathworks, #R2019a) utilizing signal processing and image processing toolboxes.

RNA sequencing: The animals and brain tissues were prepared, and then the single nuclei from the brain tissue was then obtained. Next, RNA-sequencing library preparation was performed using Chromium Next GEM Single Cell 3′ Kit v3.1, and subsequently sequenced in NovaSeq. The RNA-seq data was analyzed in R package.

Single nuclei preparation—Mice were killed and the brain tissue was dissected out. Single nuclei were prepared following the method as below: 750 ul of 30% solution was added to a 2 ml dolphin tube and add 300 μl 40% solution to the bottom of the tube. About 75 mg tissue were dounced in 700 μl Homogenization Buffer (1M Sucrose, 1M CaCl₂), 1M MgAc2, 1M Tris pH 7.8, 0.5M EDTA, 10% NP40, H2O, Beta ME (Vortex), RNase Inhibitor) with 15 strokes. Homogenate was recovered and passed through 40 um strainer, and ˜450 μl Working Solution (1M CaCl₂), 1M MgAc2, 1M Tris pH 7.8, 0.5M EDTA, H2O, Beta ME (Vortex), Optiprep) was added, and then pipetted 10 times to mix. 25% sample dilution was layered on the top, and 700 ul was pipetted to the wall of the dolphin tube to avoid bubbles. The sample was spun at 10,000 g at 4C for 5 minutes use a swinging bucket rotor with fixed angle attachment. The upper layer (˜700 μl) was removed with a pipette. 100 μl was recovered from the 30%/40% interface by looking for a nuclear pellet that may have formed on the wall of the tube slightly above the 30%/40% interface. The nuclear pellet was collected by pipetting 100 μl sample dilution, and then washed with 1 ml 0.04% BSA in PBS. A 0.04% BSA in PBS (0.2 g in 500 ml PBS) was also prepared. The nuclei were spun down at 300 g for 3 minutes at 4C. About 950 μl of supernatant was removed and 1 ml 0.04% BSA in PBS was added to wash again. The mixture was spun down at 300 g for 3 minutes at 4C and remove the supernatant, but about 50˜100 μl of supernatant was left in. Next, C-Chip was used to count the nuclei. The nuclei were resuspended before adding Trypan Blue, with the mixing volume being about 10 μl nuclei plus 10 μl Trypan Blue. The mixture was pipetted to mix well, and 20 μl of the mixture was loaded to the chip chamber. The count from the chip chamber was used to determine the dilution of the nuclei. The mixture can be diluted with 0.04% BSA if necessary. Finally, the mixture is resuspended well before adding nuclei to BSA. All the required chemicals were purchased from Sigma Aldrich. All solutions were filtered before use.

SnRNA-seq library preparation and sequencing: Once, the single nuclei was prepared, protocol Step 1 of GEM Generation & Barcoding (10× Genomics) was executed, with a target of ˜10000 nuclei/reaction. A total of 12 PCR cycles were used for the amplification of the cDNA, and 14 cycles for the Index PCR. Single cell RNA libraries were prepared using the Chromium Next GEM Single Cell 3′ Kit v3.1 according to the manufacturer's protocol (10× Genomics). The generated scRNA-seq libraries were sequenced using NovaSeq. Gene counts were obtained by aligning reads to the mouse genome.

Data Analysis

All analyses were performed in R package following the methods as described previously.

Statistics and Reproducibility

No statistical methods to predetermine or recalculate sample size were used, but the number of animals used in each experiment was based on experience and also previous publications in the field. All IHC and behavioral experiments were blinded. No data were excluded for analysis. All IHC experiments were replicated in two independent experiments of at least 3 mice per group in each experiment, and both replications was successful. For all representative images shown, images are representative of at least two independent staining and experiments. Statistical tests and significance for each experiment was calculated as noted in the appropriate figure descriptions, using t-test, Mann-Whitney test, or one-way ANOVA with a Two-stage linear step-up procedure of Benjamini, Krieger and Yekutieli post hoc analysis. Statistical significance was set at 0.05. Statistical analysis was conducted using Prism (version 9.3 and 9.7.1, GraphPad Software).

### Example 4

### INTRODUCTION

Use of Microglia Therapies in Combination with Gamma ENtrainment Using Sensory (GENUS) Stimuli for APOE4-Related Disorders.

Modifying microglia response/activation state may strengthen the ability of GENUS to clear amyloid and may improve outcomes. Ultimately, application of microglia modification in combination with GENUS to APOE4 carriers may slow the rate of progression of AD and other diseases for which APOE4 is a risk factor.

Technical Description

APOE4 Significantly Increases the Risk for Developing AD.

The mechanism underlying increased risk has been unclear. APOE is a major lipoprotein in the brain that mediates trafficking and metabolism of lipids and cholesterol. The APOE gene has three common alleles—APOE2, APOE3 and APOE4—which differ from each other by just two amino acids. Genome Wide Association Studies (GWAS) have identified APOE4 as the single strongest genetic contributor to sporadic Alzheimer's Disease (AD). Possession of a single APOE4 allele increases the risk of AD incidence 3-fold, and with two E4 alleles, 15-fold (relative to E3/E3). The APOE4 isoform has also been linked with increased levels of low density lipoprotein (LDL) and has been demonstrated to be a risk factor for cardiovascular disease and increased atherosclerosis which may have detrimental effects on brain function through decreased blood flow and altered metabolic properties. APOE4 is also associated with adverse outcomes after traumatic brain injury and Cerebral Amyloid Angiopathy (CAA).

APOE is expressed in several organs, with the highest expression in the liver, followed by the brain. In the brain, astrocytes and to some extent microglia are the major cell types that express APOE in the brain.

APOE4+ Also Increases Amyloid Load in Human Carriers.

APOE4+ individuals accumulate A #earlier than non-carriers forming earlier neurotoxic aggregates than APOE3 or APOE2. In addition, APOE4+ carriers have more tau accumulation and brain atrophy than non-carriers leading to greater memory impairment.

APOE4 May Cause AD Progression by Promoting Inflammation.

The inhibition of anti-inflammatory functions, or a combination of both. Microglia, the so-called immune cells of the brain, could become persistently activated through contact of fibrillar amyloid or other plaque-associated molecules in the temporal and frontal cortex of APOE4+ individuals. This can promote an inability to effectively remove senile plaques and lead to an extended period of inflammation that could last for years. Indeed, induced pluripotent stem cell (iPSC)-derived APOE4 microglia display impaired phagocytosis, migration and metabolic activity, as well as exacerbated cytokine secretion, and APOE4 microglia may disrupt lipid homeostasis affecting both microglia function and interaction with neurons. The examples disclosed herein suggests that APOE4 microglia may contribute to worsening AD outcomes.

Because of the heterogeneity of pathology and inflammation outcomes associated with APOE4, a single therapeutic strategy may not work for all AD patients equally. Thus, targeting a combination of APOE4-related pathogenic pathways may represent a therapeutic approach. Modifying microglia in APOE4 carriers may be one part of a therapeutic approach for APOE4 carriers.

40 Hz GENUS Improves Multiple AD Outcomes and Modifies Microglia.

Oscillations in the gamma frequency band (˜30-90 Hz) are modulated with numerous higher-order cognitive functions and are disrupted in several AD-associated mouse models, including APOE4, and human AD patients. Disclosed herein are non-invasive approaches for modifying neural activity to improve AD outcomes. The approach has been to harness patterned sensory stimuli, which are known to entrain network oscillations in humans and animal models. A 40 Hz visual and/or auditory stimulation was used in a paradigm termed Gamma ENtrainment Using Sensory stimuli (GENUS). Using this method, significant reductions in Aβ peptides and amyloid plaques were found as well as effects on microglia, astrocytes and the brain vasculature after 1 week of daily GENUS, and reduced neuroinflammation, tau phosphorylation, neurodegeneration and synapse loss when applied chronically for 3-6 weeks. The examples disclosed herein have uncovered effects of GENUS treatment on multiple microglial properties, including altered gene expression, inflammatory profile and morphology of microglia, as well as microglial Aβ colocalization and proximity to amyloid plaques, suggesting that microglia may respond to and potentially regulate the GENUS response.

In this example, an invention to intervene in a cell type specific manner, by modifying microglia in APOE4 carriers, combined with a broad therapeutic approach of GENUS, which modifies multiple cell and pathway readouts, is proposed.

Given that APOE4 has known defects in microglia, including aberrant inflammatory activity, it was hypothesized that APOE4 carriers may have altered response to GENUS. Therefore mouse models of AD with human APOE4 knocked in were investigated.

In order to determine if APOE allele status could modify neuronal cells change or entrainment in the mouse models, neuronal cells were counted, and cranial electrophysiology (EEG) was used to test APOE3 and APOE4 in the 5×FAD background (FIGS. 31A-31B). The data herein confirmed thatAPOE4-KI (knock in) mice in an AD background were capable of entraining at 40 Hz.

Using the APOE4 human knock in mouse models expressing amyloid pathology (5×FAD) or tau pathology (P301S), mice were treated chronically using 3 weeks with GENUS auditory and visual (A+V) 40 Hz flickers stimulation.

APOE4 Animal Models Beneficially Respond to GENUS in Non-Amyloid Models to Increase Neuroprotection.

GENUS has been shown to improve neuronal protection in a tau model of AD. This mouse model was examined with human APOE4 knocked into the mouse locus (APOE4-KI) to determine if APOE4 genotype interfered with the neuronal protection afforded by GENUS. 9-10 month old APOE3 Tau and APOE4 Tau male mice were treated with 21 days of auditory and visual combined (A+V) GENUS. A significant neuronal protection was observed in both APOE3 and APOE4 tau model mice in the hippocampus (FIGS. 32A-32B), particularly in the CA3 subregion (FIG. 32B).

Next, whether APOE4-KI tau animals showed differential microglial response to GENUS was examined. Hippocampal mouse brain sections were stained for Iba1, a microglia/macrophage specific marker, and cell numbers were counted. In the APOE tau model, microglia numbers were reduced in the GENUS treated group for both APOE3 and APOE4 compared to the control group that did not receive GENUS (FIGS. 33A-33B).

Example 4 suggests that APOE4 animals may be capable of sensing and responding to GENUS stimulation, and in tau models show significant neuronal protection and likely reduced inflammation (reduced microglia).

APOE4 Response to GENUS May be Attenuated in an Amyloid Model.

Given that APOE4+ individuals accumulate A #earlier than non-carriers, and that APOE facilitates the response of microglia to amyloid, GENUS outcomes in an APOE4-KI amyloid model were examined. To this end, 21d A+V (audio and visual) GENUS in APOE-KI 5×FAD model was performed and amyloid and microglial outcomes were examined.

In 7-9 month old male APOE4-KI 5×FAD mice, following 21 days of A+V GENUS at 40 Hz, it was found that amyloid load was not reduced (FIGS. 34A-34B), contrary to other non-APOE4 amyloid models. These data were repeated in multiple APOE4-KI 5×FAD cohorts. Preliminary data in APOE3-KI 5×FAD animals suggests that amyloid is reduced in this genotype (FIGS. 35A-35B), suggesting that APOE4-KI animals have aberrant response to GENUS in the context of amyloid.

An independent cohort of 6 month old animals on a different control diet (based on AIN76A) showed similar outcomes, where APOE3 5×FAD animals tended to have reduced amyloid plaques while APOE4 5×FAD animals showed no reduction in plaques (FIG. 36).

Together these data suggest that APOE4-KI animals show deficient response to GENUS with respect to amyloid clearance. Given that APOE facilitates the microglial response to amyloid, it was reasoned that APOE4-KI microglia may be at least partially responsible for this aberrant outcome. Therefore, it was sought to reduce microglia in APOE4-KI animals by using the CSF1r inhibitor PLX3397, which has been shown to have beneficial outcomes in an APOE4-KI tau model.

Next, 7-8 month old male APOE4-KI 5×FAD animals were treated with 3 weeks of PLX3397 diet (or control), followed by 21 days of A+V GENUS, during which animals remained on their diets (FIG. 37).

GENUS Induced Amyloid Clearance is Restored in APOE4 KI Animals when Microglia are Depleted.

It was observed that animals who received the microglia depleting PLX3397 showed significant reduction in microglia numbers (FIGS. 38A-38B).

This reduction in microglia did not in and of itself result in a significant change to amyloid load as shown in FIGS. 39A-39B.

However, when the effect of microglia depletion in combination with 21 days A+V GENUS was examined, a significant reduction in plaque numbers (FIGS. 40A-40B), as well as a reduction in mean intensity and total area (FIGS. 41A-41B), was observed.

Then it was asked if the depletion of microglia rendered the microglia that remained more responsive to GENUS, as observed in the APOE4-KI tau model. In order to do this, it was asked whether microglia number was further altered by GENUS stimulation following PLX3397 diet. It was found that the remaining microglia number were not altered by GENUS status (FIG. 42), suggesting that the depletion of microglia was allowing for GENUS-mediated clearance of amyloid, but the remaining microglia population may not be responding to GENUS.

Altogether the data disclosed herein suggests thatAPOE4-KI animals may display an amyloid specific aberrant response to GENUS, where in the absence of amyloid neuroprotection is observed following GENUS; but in the presence of amyloid, an attenuated GENUS response is observed, including failure to clear amyloid. This effect may be mediated in part by dysfunctional APOE4 microglia, and the depletion of microglia in APOE4-KI 5×FAD animals may improve GENUS-mediated amyloid clearance. These data disclosed herein suggests a combinatorial approach to treating AD in APOE4-carriers may result in improved outcomes.

In summary, the examples disclosed herein identify a means of improving certain therapeutic approaches, such as GENUS, in APOE4 carriers by modifying microglia. By using cell type targeting or anti-inflammatory molecules/drugs in combination with GENUS, APOE4-carriers may be more receptive to the beneficial outcomes associated with GENUS therapy.

The approach disclosed herein is unique in that it unites two previously unconnected therapeutic approaches (microglia modification and GENUS therapy), with particularly enhanced benefits for APOE4-carriers, who form a large proportion of the AD population and suffer cell-type specific dysfunction that may interfere with therapeutic outcomes.

The finding that APOE4 microglia may impede GENUS mediated amyloid clearance has significant relevance to the treatment of APOE4-specific disease pathologies. Indeed, while studies have focused on AD relevant phenotypes, it is reasonable to hypothesize that the microglia dysregulation observed in the mouse models disclosed herein would be true for any cell/tissue expressing or requiring APOE function. Indeed, as mentioned above, APOE4 is associated with multiple disorders across a range of tissues, including Cerebral Amyloid Angiopathy (CAA) and recovery from traumatic brain injury (TBI). Combinatorial therapies such as the ones disclosed herein in these contexts may reduce pathologies induced by APOE4 across multiple tissue types.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for increasing phase locking of neurons to gamma oscillations in at least one brain region of a subject for treating Alzheimer's disease in the subject in need thereof, the method comprising:
administering an inhibitor including a colony-stimulating factor-1 receptor (CSF1R) inhibitor or a colony-stimulating factor-1 (CSF1) inhibitor to the subject; and
administering a stimulus to the subject having a frequency from about 20 Hz to about 60 Hz.

2. The method of claim 1, the administering the inhibitor further comprising administering a CSF1R inhibitor, wherein the CSF1R inhibitor is pexidartinib.

3. The method of claim 1, the administering the inhibitor further comprising administering a CSF1R inhibitor, wherein the CSF1R inhibitor is selected from the group consisting of pexidartinib, bosutinib, imatinib, gefitinib, ruxolitinib, dasatinib, sunitinib, erlotinib, lapatinib, pazopanib, crizotinib, vemurafenib, PLX7486, ARRY-382, Edicotinib, BLZ945, Emactuzumab, AMG 820, Cabiralizumab, and IMC-CS4.

4. The method of claim 1, the administering the inhibitor further comprising administering a CSF1 inhibitor, wherein the CSF1 inhibitor is selected from the group consisting of PD-0360324 and MCS110.

5. The method of claim 1, wherein the frequency of the stimulus is about 40 Hz.

6. The method of claim 1, the administering the inhibitor including initiating administering the inhibitor prior to the non-invasively administering the stimulus.

7. The method of claim 6, the administering the inhibitor including administering the inhibitor for at least 20 days prior to the non-invasively administering the stimulus.

8. The method of claim 6, the administering the inhibitor including continuing to administer the inhibitor during the non-invasively administering the stimulus.

9. The method of claim 8, the administering the stimulus including non-invasively administering the stimulus for at least 30 days.

10. The method of claim 8, the administering the stimulus including non-invasively administering the stimulus for at least one hour per day.

11. The method of claim 1, the administering the stimulus including non-invasively administering the stimulus for at least 30 days.

12. The method of claim 11, the administering the stimulus including non-invasively administering the stimulus for at least one hour per day.

13. The method of claim 1, the administering the stimulus including non-invasively administering the stimulus for at least one hour per day.

14. The method of claim 1, wherein the at least one brain region includes at least one of the visual cortex and the hippocampus.

15. The method of claim 1, wherein the subject has at least one Apolipoprotein E4 (APOE4) allele.

16. A method for increasing phase locking of neurons to gamma oscillations in at least one brain region of a subject, the subject having been administered an inhibitor including a colony-stimulating factor-1 receptor (CSF1R) inhibitor or a colony-stimulating factor-1 (CSF1) inhibitor, the method comprising:
administering a stimulus to the subject having a frequency from about 20 Hz to about 60 Hz.

17. The method of claim 16, wherein the frequency of the stimulus is about 40 Hz.

18. The method of claim 16, the administering the stimulus including non-invasively administering the stimulus for at least 30 days.

19. The method of claim 16, the administering the stimulus including non-invasively administering the stimulus for at least one hour per day.

20. The method of claim 16, wherein the at least one brain region includes at least one of the visual cortex and the hippocampus.

21. A method, comprising:
providing a device that administers a stimulus to a subject during use of the device, the subject having been administered an inhibitor including a colony-stimulating factor-1 receptor (CSF1R) inhibitor or a colony-stimulating factor-1 (CSF1) inhibitor, wherein the stimulus has a frequency of from about 20 Hz to about 60 Hz.

22. The method of claim 21, wherein the frequency is about 40 Hz.

23-50. (canceled)
